SEQUENCE LISTING (200 sequences)

SEQ ID NO: 1-11 - skipped (11 entries)

SEQ ID NO: 12 - protein, 17 aa, synthetic construct
  VARIANT 6: note = N or Q
  VARIANT 13: note = Q or E
EINPIXGRSNYAXKFQG

SEQ ID NO: 13 - protein, 11 aa, synthetic construct
  VARIANT 8: note = N or S
RTSENIYXNLA

SEQ ID NO: 14 - protein, 7 aa, synthetic construct
  VARIANT 2: note = A or G
  VARIANT 7: note = D or E
AXTNLAX

SEQ ID NO: 15 - protein, 10 aa, synthetic construct
  VARIANT 10: note = Residue may be deleted
QHFWGTPLTF

SEQ ID NO: 16 - protein, 7 aa, synthetic construct
  VARIANT 7: note = D or E
AATNLAX

SEQ ID NO: 17 - protein, 9 aa, synthetic construct
YTFTNYWMH

SEQ ID NO: 18 - protein, 17 aa, synthetic construct
EINPINGRSNYAQKFQG

SEQ ID NO: 19 - protein, 7 aa, synthetic construct
GTRAMHY

SEQ ID NO: 20 - protein, 17 aa, synthetic construct
EINPINGRSNYAEKFQG

SEQ ID NO: 21 - protein, 17 aa, synthetic construct
EINPIQGRSNYAEKFQG

SEQ ID NO: 22 - protein, 11 aa, synthetic construct
RTSENIYNNLA

SEQ ID NO: 23 - protein, 7 aa, synthetic construct
AATNLAD

SEQ ID NO: 24 - protein, 9 aa, synthetic construct
QHFWGTPLT

SEQ ID NO: 25 - protein, 7 aa, synthetic construct
AATNLAE

SEQ ID NO: 26 - protein, 10 aa, synthetic construct
QHFWGTPLTF

SEQ ID NO: 27 - protein, 11 aa, synthetic construct
RTSENIYSNLA

SEQ ID NO: 28 - protein, 7 aa, synthetic construct
AGTNLAD

SEQ ID NO: 29 - protein, 116 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 30 - protein, 116 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 31 - protein, 116 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 32 - protein, 116 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 33 - protein, 116 aa, synthetic construct
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 34 - protein, 107 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 35 - protein, 107 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 36 - protein, 107 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 37 - protein, 107 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

SEQ ID NO: 38 - protein, 107 aa, synthetic construct
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 39 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 214 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 214 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 5 aa, synthetic construct
NYWMH

SEQ ID NO: 68-69 - skipped (2 entries)

SEQ ID NO: 70 - protein, 4 aa, synthetic construct
GGFG

SEQ ID NO: 71 - protein, 4 aa, synthetic construct
ALAL

SEQ ID NO: 72 - protein, 4 aa, synthetic construct
GFLG

SEQ ID NO: 73-79 - skipped (7 entries)

SEQ ID NO: 80 - DNA, 19 bp, synthetic construct
  source 1..19: mol_type = other DNA
tctaaagggcagcatttgt

SEQ ID NO: 81 - DNA, 20 bp, synthetic construct
  source 1..20: mol_type = other DNA
ggagatggcagtttccttag

SEQ ID NO: 82 - DNA, 25 bp, synthetic construct
  source 1..25: mol_type = other DNA
tcagccagtgaagctcctactcaga

SEQ ID NO: 83-85 - skipped (3 entries)

SEQ ID NO: 86 - DNA, 21 bp, synthetic construct
  source 1..21: mol_type = other DNA
gcagttcaagctaaacaaccg

SEQ ID NO: 87 - DNA, 18 bp, synthetic construct
  source 1..18: mol_type = other DNA
ttgttgccttcactggct

SEQ ID NO: 88 - DNA, 28 bp, synthetic construct
  source 1..28: mol_type = other DNA
tgtctaaagggcagcatttgtacaagga

SEQ ID NO: 89-99 - skipped (11 entries)

SEQ ID NO: 100 - DNA, 28 bp, synthetic construct
  source 1..28: mol_type = other DNA
  modified_base 1..28: mod_base = OTHER; note = phosphorodiamidate morpholino oligonucleotide
gagctcagatcttctaacttcctcttca

SEQ ID NO: 101 - DNA, 28 bp, synthetic construct
  source 1..28: mol_type = other DNA
  modified_base 1..28: mod_base = OTHER; note = phosphorodiamidate morpholino oligonucleotide
cagagctcagatcttctaacttcctctt

SEQ ID NO: 102 - DNA, 28 bp, synthetic construct
  source 1..28: mol_type = other DNA
  modified_base 1..28: mod_base = OTHER; note = phosphorodiamidate morpholino oligonucleotide
ctcagagctcagatcttctaacttcctc

SEQ ID NO: 103 - DNA, 28 bp, synthetic construct
  source 1..28: mol_type = other DNA
  modified_base 1..28: mod_base = OTHER; note = phosphorodiamidate morpholino oligonucleotide
cactcagagctcagatcttctaacttcc

SEQ ID NO: 104 - DNA, 28 bp, synthetic construct
  source 1..28: mol_type = other DNA
  modified_base 1..28: mod_base = OTHER; note = phosphorodiamidate morpholino oligonucleotide
tccactcagagctcagatcttctaactt

SEQ ID NO: 105 - DNA, 28 bp, synthetic construct
  source 1..28: mol_type = other DNA
  modified_base 1..28: mod_base = OTHER; note = phosphorodiamidate morpholino oligonucleotide
cttccactcagagctcagatcttctaac

SEQ ID NO: 106 - DNA, 28 bp, synthetic construct
  source 1..28: mol_type = other DNA
  modified_base 1..28: mod_base = OTHER; note = phosphorodiamidate morpholino oligonucleotide
gccttccactcagagctcagatcttcta

SEQ ID NO: 107 - DNA, 28 bp, synthetic construct
  source 1..28: mol_type = other DNA
  modified_base 1..28: mod_base = OTHER; note = phosphorodiamidate morpholino oligonucleotide
ccgccttccactcagagctcagatcttc

SEQ ID NO: 108 - DNA, 28 bp, synthetic construct
  source 1..28: mol_type = other DNA
  modified_base 1..28: mod_base = OTHER; note = phosphorodiamidate morpholino oligonucleotide
taccgccttccactcagagctcagatct

SEQ ID NO: 109 - DNA, 28 bp, synthetic construct
  source 1..28: mol_type = other DNA
  modified_base 1..28: mod_base = OTHER; note = phosphorodiamidate morpholino oligonucleotide
tttaccgccttccactcagagctcagat

SEQ ID NO: 110 - DNA, 28 bp, synthetic construct
  source 1..28: mol_type = other DNA
  modified_base 1..28: mod_base = OTHER; note = phosphorodiamidate morpholino oligonucleotide
ggtttaccgccttccactcagagctcag

SEQ ID NO: 111 - DNA, 28 bp, synthetic construct
  source 1..28: mol_type = other DNA
  modified_base 1..28: mod_base = OTHER; note = phosphorodiamidate morpholino oligonucleotide
acggtttaccgccttccactcagagctc

SEQ ID NO: 112 - DNA, 28 bp, synthetic construct
  source 1..28: mol_type = other DNA
  modified_base 1..28: mod_base = OTHER; note = phosphorodiamidate morpholino oligonucleotide
aaacggtttaccgccttccactcagagc

SEQ ID NO: 113 - DNA, 28 bp, synthetic construct
  source 1..28: mol_type = other DNA
  modified_base 1..28: mod_base = OTHER; note = phosphorodiamidate morpholino oligonucleotide
gtaaacggtttaccgccttccactcaga

SEQ ID NO: 114 - DNA, 28 bp, synthetic construct
  source 1..28: mol_type = other DNA
  modified_base 1..28: mod_base = OTHER; note = phosphorodiamidate morpholino oligonucleotide
aagtaaacggtttaccgccttccactca

SEQ ID NO: 115 - DNA, 28 bp, synthetic construct
  source 1..28: mol_type = other DNA
  modified_base 1..28: mod_base = OTHER; note = phosphorodiamidate morpholino oligonucleotide
tgaagtaaacggtttaccgccttccact

SEQ ID NO: 116 - DNA, 28 bp, synthetic construct
  source 1..28: mol_type = other DNA
  modified_base 1..28: mod_base = OTHER; note = phosphorodiamidate morpholino oligonucleotide
cttgaagtaaacggtttaccgccttcca

SEQ ID NO: 117 - DNA, 28 bp, synthetic construct
  source 1..28: mol_type = other DNA
  modified_base 1..28: mod_base = OTHER; note = phosphorodiamidate morpholino oligonucleotide
ctcttgaagtaaacggtttaccgccttc

SEQ ID NO: 118 - DNA, 28 bp, synthetic construct
  source 1..28: mol_type = other DNA
  modified_base 1..28: mod_base = OTHER; note = phosphorodiamidate morpholino oligonucleotide
agctcttgaagtaaacggtttaccgcct

SEQ ID NO: 119 - DNA, 28 bp, synthetic construct
  source 1..28: mol_type = other DNA
  modified_base 1..28: mod_base = OTHER; note = phosphorodiamidate morpholino oligonucleotide
tcagctcttgaagtaaacggtttaccgc

SEQ ID NO: 120 - DNA, 28 bp, synthetic construct
  source 1..28: mol_type = other DNA
  modified_base 1..28: mod_base = OTHER; note = phosphorodiamidate morpholino oligonucleotide
cctcagctcttgaagtaaacggtttacc

SEQ ID NO: 121 - DNA, 28 bp, synthetic construct
  source 1..28: mol_type = other DNA
  modified_base 1..28: mod_base = OTHER; note = phosphorodiamidate morpholino oligonucleotide
gccctcagctcttgaagtaaacggttta

SEQ ID NO: 122 - DNA, 28 bp, synthetic construct
  source 1..28: mol_type = other DNA
  modified_base 1..28: mod_base = OTHER; note = phosphorodiamidate morpholino oligonucleotide
ttgccctcagctcttgaagtaaacggtt

SEQ ID NO: 123 - DNA, 28 bp, synthetic construct
  source 1..28: mol_type = other DNA
  modified_base 1..28: mod_base = OTHER; note = phosphorodiamidate morpholino oligonucleotide
ctttgccctcagctcttgaagtaaacgg

SEQ ID NO: 124 - DNA, 28 bp, synthetic construct
  source 1..28: mol_type = other DNA
  modified_base 1..28: mod_base = OTHER; note = phosphorodiamidate morpholino oligonucleotide
tgctttgccctcagctcttgaagtaaac

SEQ ID NO: 125 - DNA, 28 bp, synthetic construct
  source 1..28: mol_type = other DNA
  modified_base 1..28: mod_base = OTHER; note = phosphorodiamidate morpholino oligonucleotide
gctgctttgccctcagctcttgaagtaa

SEQ ID NO: 126 - DNA, 28 bp, synthetic construct
  source 1..28: mol_type = other DNA
  modified_base 1..28: mod_base = OTHER; note = phosphorodiamidate morpholino oligonucleotide
aggctgctttgccctcagctcttgaagt

SEQ ID NO: 127 - DNA, 28 bp, synthetic construct
  source 1..28: mol_type = other DNA
  modified_base 1..28: mod_base = OTHER; note = phosphorodiamidate morpholino oligonucleotide
tcaggctgctttgccctcagctcttgaa

SEQ ID NO: 128 - DNA, 28 bp, synthetic construct
  source 1..28: mol_type = other DNA
  modified_base 1..28: mod_base = OTHER; note = phosphorodiamidate morpholino oligonucleotide
ggtcaggctgctttgccctcagctcttg

SEQ ID NO: 129 - DNA, 28 bp, synthetic construct
  source 1..28: mol_type = other DNA
  modified_base 1..28: mod_base = OTHER; note = phosphorodiamidate morpholino oligonucleotide
taggtcaggctgctttgccctcagctct

SEQ ID NO: 130 - DNA, 28 bp, synthetic construct
  source 1..28: mol_type = other DNA
  modified_base 1..28: mod_base = OTHER; note = phosphorodiamidate morpholino oligonucleotide
gctaggtcaggctgctttgccctcagct

SEQ ID NO: 131 - DNA, 28 bp, synthetic construct
  source 1..28: mol_type = other DNA
  modified_base 1..28: mod_base = OTHER; note = phosphorodiamidate morpholino oligonucleotide
gagctaggtcaggctgctttgccctcag

SEQ ID NO: 132 - DNA, 28 bp, synthetic construct
  source 1..28: mol_type = other DNA
  modified_base 1..28: mod_base = OTHER; note = phosphorodiamidate morpholino oligonucleotide
aggagctaggtcaggctgctttgccctc

SEQ ID NO: 133 - DNA, 28 bp, synthetic construct
  source 1..28: mol_type = other DNA
  modified_base 1..28: mod_base = OTHER; note = phosphorodiamidate morpholino oligonucleotide
ccaggagctaggtcaggctgctttgccc

SEQ ID NO: 134 - DNA, 28 bp, synthetic construct
  source 1..28: mol_type = other DNA
  modified_base 1..28: mod_base = OTHER; note = phosphorodiamidate morpholino oligonucleotide
gtccaggagctaggtcaggctgctttgc

SEQ ID NO: 135 - DNA, 28 bp, synthetic construct
  source 1..28: mol_type = other DNA
  modified_base 1..28: mod_base = OTHER; note = phosphorodiamidate morpholino oligonucleotide
cagtccaggagctaggtcaggctgcttt

SEQ ID NO: 136 - DNA, 28 bp, synthetic construct
  source 1..28: mol_type = other DNA
  modified_base 1..28: mod_base = OTHER; note = phosphorodiamidate morpholino oligonucleotide
gtcagtccaggagctaggtcaggctgct

SEQ ID NO: 137 - DNA, 28 bp, synthetic construct
  source 1..28: mol_type = other DNA
  modified_base 1..28: mod_base = OTHER; note = phosphorodiamidate morpholino oligonucleotide
tggtcagtccaggagctaggtcaggctg

SEQ ID NO: 138 - DNA, 28 bp, synthetic construct
  source 1..28: mol_type = other DNA
  modified_base 1..28: mod_base = OTHER; note = phosphorodiamidate morpholino oligonucleotide
agtggtcagtccaggagctaggtcaggc

SEQ ID NO: 139 - DNA, 28 bp, synthetic construct
  source 1..28: mol_type = other DNA
  modified_base 1..28: mod_base = OTHER; note = phosphorodiamidate morpholino oligonucleotide
atagtggtcagtccaggagctaggtcag

SEQ ID NO: 140 - DNA, 28 bp, synthetic construct
  source 1..28: mol_type = other DNA
  modified_base 1..28: mod_base = OTHER; note = phosphorodiamidate morpholino oligonucleotide
caatagtggtcagtccaggagctaggtc

SEQ ID NO: 141 - DNA, 28 bp, synthetic construct
  source 1..28: mol_type = other DNA
  modified_base 1..28: mod_base = OTHER; note = phosphorodiamidate morpholino oligonucleotide
tccaatagtggtcagtccaggagctagg

SEQ ID NO: 142 - DNA, 28 bp, synthetic construct
  source 1..28: mol_type = other DNA
  modified_base 1..28: mod_base = OTHER; note = phosphorodiamidate morpholino oligonucleotide
gctccaatagtggtcagtccaggagcta

SEQ ID NO: 143 - DNA, 28 bp, synthetic construct
  source 1..28: mol_type = other DNA
  modified_base 1..28: mod_base = OTHER; note = phosphorodiamidate morpholino oligonucleotide
aggctccaatagtggtcagtccaggagc

SEQ ID NO: 144 - DNA, 22 bp, synthetic construct
  source 1..22: mol_type = other DNA
  modified_base 1..22: mod_base = OTHER; note = phosphorodiamidate morpholino oligonucleotide
ccgccttccactcagagctcag

SEQ ID NO: 145 - DNA, 22 bp, synthetic construct
  source 1..22: mol_type = other DNA
  modified_base 1..22: mod_base = OTHER; note = phosphorodiamidate morpholino oligonucleotide
taccgccttccactcagagctc

SEQ ID NO: 146 - DNA, 22 bp, synthetic construct
  source 1..22: mol_type = other DNA
  modified_base 1..22: mod_base = OTHER; note = phosphorodiamidate morpholino oligonucleotide
gtcagtccaggagctaggtcag

SEQ ID NO: 147 - DNA, 22 bp, synthetic construct
  source 1..22: mol_type = other DNA
  modified_base 1..22: mod_base = OTHER; note = phosphorodiamidate morpholino oligonucleotide
tggtcagtccaggagctaggtc

SEQ ID NO: 148 - DNA, 24 bp, synthetic construct
  source 1..24: mol_type = other DNA
  modified_base 1..24: mod_base = OTHER; note = phosphorodiamidate morpholino oligonucleotide
cttccactcagagctcagatcttc

SEQ ID NO: 149 - DNA, 24 bp, synthetic construct
  source 1..24: mol_type = other DNA
  modified_base 1..24: mod_base = OTHER; note = phosphorodiamidate morpholino oligonucleotide
gccttccactcagagctcagatct

SEQ ID NO: 150 - DNA, 24 bp, synthetic construct
  source 1..24: mol_type = other DNA
  modified_base 1..24: mod_base = OTHER; note = phosphorodiamidate morpholino oligonucleotide
ccgccttccactcagagctcagat

SEQ ID NO: 151 - DNA, 24 bp, synthetic construct
  source 1..24: mol_type = other DNA
  modified_base 1..24: mod_base = OTHER; note = phosphorodiamidate morpholino oligonucleotide
taccgccttccactcagagctcag

SEQ ID NO: 152 - DNA, 24 bp, synthetic construct
  source 1..24: mol_type = other DNA
  modified_base 1..24: mod_base = OTHER; note = phosphorodiamidate morpholino oligonucleotide
tttaccgccttccactcagagctc

SEQ ID NO: 153 - DNA, 24 bp, synthetic construct
  source 1..24: mol_type = other DNA
  modified_base 1..24: mod_base = OTHER; note = phosphorodiamidate morpholino oligonucleotide
ggtttaccgccttccactcagagc

SEQ ID NO: 154 - DNA, 24 bp, synthetic construct
  source 1..24: mol_type = other DNA
  modified_base 1..24: mod_base = OTHER; note = phosphorodiamidate morpholino oligonucleotide
acggtttaccgccttccactcaga

SEQ ID NO: 155 - DNA, 24 bp, synthetic construct
  source 1..24: mol_type = other DNA
  modified_base 1..24: mod_base = OTHER; note = phosphorodiamidate morpholino oligonucleotide
cagtccaggagctaggtcaggctg

SEQ ID NO: 156 - DNA, 24 bp, synthetic construct
  source 1..24: mol_type = other DNA
  modified_base 1..24: mod_base = OTHER; note = phosphorodiamidate morpholino oligonucleotide
gtcagtccaggagctaggtcaggc

SEQ ID NO: 157 - DNA, 24 bp, synthetic construct
  source 1..24: mol_type = other DNA
  modified_base 1..24: mod_base = OTHER; note = phosphorodiamidate morpholino oligonucleotide
tggtcagtccaggagctaggtcag

SEQ ID NO: 158 - DNA, 24 bp, synthetic construct
  source 1..24: mol_type = other DNA
  modified_base 1..24: mod_base = OTHER; note = phosphorodiamidate morpholino oligonucleotide
agtggtcagtccaggagctaggtc

SEQ ID NO: 159 - DNA, 24 bp, synthetic construct
  source 1..24: mol_type = other DNA
  modified_base 1..24: mod_base = OTHER; note = phosphorodiamidate morpholino oligonucleotide
atagtggtcagtccaggagctagg

SEQ ID NO: 160 - DNA, 24 bp, synthetic construct
  source 1..24: mol_type = other DNA
  modified_base 1..24: mod_base = OTHER; note = phosphorodiamidate morpholino oligonucleotide
caatagtggtcagtccaggagcta

SEQ ID NO: 161 - DNA, 26 bp, synthetic construct
  source 1..26: mol_type = other DNA
  modified_base 1..26: mod_base = OTHER; note = phosphorodiamidate morpholino oligonucleotide
gccttccactcagagctcagatcttc

SEQ ID NO: 162 - DNA, 26 bp, synthetic construct
  source 1..26: mol_type = other DNA
  modified_base 1..26: mod_base = OTHER; note = phosphorodiamidate morpholino oligonucleotide
ccgccttccactcagagctcagatct

SEQ ID NO: 163 - DNA, 26 bp, synthetic construct
  source 1..26: mol_type = other DNA
  modified_base 1..26: mod_base = OTHER; note = phosphorodiamidate morpholino oligonucleotide
taccgccttccactcagagctcagat

SEQ ID NO: 164 - DNA, 26 bp, synthetic construct
  source 1..26: mol_type = other DNA
  modified_base 1..26: mod_base = OTHER; note = phosphorodiamidate morpholino oligonucleotide
tttaccgccttccactcagagctcag

SEQ ID NO: 165 - DNA, 26 bp, synthetic construct
  source 1..26: mol_type = other DNA
  modified_base 1..26: mod_base = OTHER; note = phosphorodiamidate morpholino oligonucleotide
ggtttaccgccttccactcagagctc

SEQ ID NO: 166 - DNA, 26 bp, synthetic construct
  source 1..26: mol_type = other DNA
  modified_base 1..26: mod_base = OTHER; note = phosphorodiamidate morpholino oligonucleotide
gtcagtccaggagctaggtcaggctg

SEQ ID NO: 167 - DNA, 26 bp, synthetic construct
  source 1..26: mol_type = other DNA
  modified_base 1..26: mod_base = OTHER; note = phosphorodiamidate morpholino oligonucleotide
tggtcagtccaggagctaggtcaggc

SEQ ID NO: 168 - DNA, 26 bp, synthetic construct
  source 1..26: mol_type = other DNA
  modified_base 1..26: mod_base = OTHER; note = phosphorodiamidate morpholino oligonucleotide
agtggtcagtccaggagctaggtcag

SEQ ID NO: 169 - DNA, 26 bp, synthetic construct
  source 1..26: mol_type = other DNA
  modified_base 1..26: mod_base = OTHER; note = phosphorodiamidate morpholino oligonucleotide
atagtggtcagtccaggagctaggtc

SEQ ID NO: 170-199 - skipped (30 entries)

SEQ ID NO: 200 - RNA, 109 nt, Homo sapiens
  source 1..109: mol_type = mRNA
aggaagttagaagatctgagctctgagtggaaggcggtaaaccgtttacttcaagagctgagggcaaagcagcctgacctagctcctggactgaccactattggagcct

(19) United States
(12) Patent Application Publication
(10) Pub. No.: US 2025/0325693 A1
(43) Pub. Date: Oct. 23, 2025

(54) ANTIBODY-OLIGONUCLEOTIDE CONJUGATE COMPOSITIONS AND METHODS OF INDUCING DMD EXON 50 SKIPPING

(71) Applicant: Avidity Biosciences, Inc., San Diego, CA (US)

(72) Inventors: Tyler ALBIN, Encinitas, CA (US); Michael Caramian COCHRAN, Palo Alto, CA (US); Venkata Ramana DOPPALAPUDI, San Diego, CA (US); Usue ETXANIZ IRIGOIEN, San Diego, CA (US); Georgios KARAMANLIDIS, Thousand Oaks, CA (US); Isaac MARKS, San Diego, CA (US); Sven Ove Kristoffer SVENSSON, San Diego, CA (US)

(21) Appl. No.: 19/097,573

(22) Filed: Apr. 1, 2025

Related U.S. Application Data
(60) Provisional application No. 63/574,108, filed on Apr. 3, 2024.

(51) Int. Cl. A61K 47/68; A61P 21/00

(52) U.S. Cl. CPC A61K 47/6807; A61K 47/6843; A61K 47/6889; A61P 21/00

(57) ABSTRACT

Disclosed herein are antibody-oligonucleotide conjugates and pharmaceutical compositions comprising the antibody-oligonucleotide conjugates that induce an alteration in an incorrectly spliced dystrophin mRNA transcript to induce DMD exon 50 skipping. Also described herein include methods for treating muscle dystrophy including Duchenne muscular dystrophy by administering antibody-oligonucleotide conjugates or a pharmaceutical composition comprising the antibody-oligonucleotide conjugates that induce an alteration in an incorrectly spliced dystrophin mRNA transcript to induce DMD exon 50 skipping.

### CROSS REFERENCE

This application claims the benefit of U.S. Provisional Application No. 63/574,108, filed Apr. 3, 2024, which is incorporated herein by reference in its entirety.

### SEQUENCE LISTING

This application contains a Sequence Listing which has been submitted electronically in XML format. The Sequence Listing XML is incorporated herein by reference. Said XML file, created on Apr. 21, 2025, is named 45532-780_201_SL.xml and is 187.639 bytes in size.

### BACKGROUND OF THE DISCLOSURE

Duchenne Muscular Dystrophy (DMD) is a rare X-linked neuromuscular disease that manifests primarily in boys, affecting about 1:5000-10.000 males born worldwide. There are about 300.000 DMD patients worldwide. DMD is a monogenic disease: it is progressive, severe and irreversible. The disease is caused by mutations in the DMD gene, the longest gene in the human genome (79) exons), which encodes for the dystrophin protein (430 kDa). The central domain of dystrophin, called rod domain, is formed by 24 spectrin-like repeats that function as a shock-absorber and protect the sarcolemma from damage during movement.

DMD is caused by mutations (changes) within the dystrophin gene. Deletions of one or more exons are the most common type of mutation. Since there are a total of 79 exons in the dystrophin gene, there are many different deletions that can occur. However, there are certain areas of the gene that are more likely to have a deletion, and these areas are called “hot spots”. The deletions in the DMD gene that are non-randomly distributed with many of the large gene deletions that occur in the DMD gene can be detected in specific hotspot areas of the gene. These hotspots are clustered within two main regions: about 20% of the deletions occur at the 5′ proximal portion of the gene (exons 1, 3, 4, 5, 8, 13, 19); and about 80% of the deletions occur at the mid-distal region i.e. 42-45, 47, 48, 50-53 (Den Dunnen et al. Am J Hum Genet. 1989: 45 (6); 835-847). The mutated DMD gene fails to produce any functional dystrophin and lack of functional dystrophin results in progressive muscle weakness due to muscle injury, repair, inflammation changes and paralysis.

Current research for DMD therapy includes stem cell replacement therapy, analog up-regulation, gene replacement, and exon-skipping technology. Exon-skipping technology uses structural analogs of DNA called antisense oligonucleotides to help cells skip over a specific exon during RNA splicing. These antisense oligonucleotides allow faulty parts of the dystrophin gene to be skipped over when it is transcribed to RNA for protein production, permitting a still-truncated but more functional version of the dystrophin protein to be produced by the muscle cells.

There are several antisense oligonucleotides that have already been approved for DMD patients with amenable to exon 45, 51, or 53 skipping. The antisense oligonucleotide named Eteplirsen has been approved in the United States for the treatment of mutations amenable to dystrophin exon 51 skipping. The antisense oligonucleotide named Golodirsen was approved for medical use in the United States in 2019, for the treatment of cases that can benefit from skipping exon 53 of the dystrophin transcript. The antisense oligonucleotide named Casimersen was approved for treatment in the United States in February 2021 for patients who have a confirmed mutation of the DMD gene that is amenable to exon 45 skipping.

Despite extensive research using exon skipping for exon 50, there is no FDA approved exon skipping therapy for DMD patients amenable to exon 50 skipping. Approximately 4% of the DMD patient population are amenable to exon 50 skipping and the majority of these DMD patients may also have a deletion of exon 51 of the DMD transcript.

A new class of therapeutics called antibody oligonucleotide conjugates (AOC) improves the delivery of antisense oligonucleotides. These AOCs target and deliver antisense oligonucleotides to specific tissue and cell types including muscle cells. These AOCs are being developed for the potential breakthrough therapy for DMD patients including patients that are amenable to exon 50 skipping. There is a need to provide improved therapy for DMD patients amenable to exon 50 skipping.

### SUMMARY OF THE DISCLOSURE

Disclosed herein, in certain aspects, are phosphorodiamidate morpholino oligonucleotide (PMO) conjugates comprising an anti-transferrin receptor antibody or antigen binding fragment thereof conjugated to a PMO molecule that hybridizes to a pre-mRNA transcript of the DMI) gene and induces exon 50 skipping in said pre-mRNA transcript to generate a mRNA transcript encoding a truncated dystrophin protein. In some aspects, the PMO molecule hybridizes to a site within an exon, an acceptor splice site, a donor splice site, or an exonic splice enhancer element of a pre-mRNA transcript of the DMD gene. In some aspects, the PMO molecule comprises from about 10 to about 30 nucleotides in length. In some aspects, the PMO molecule comprises from about 10 to about 26 nucleotides in length. In some aspects, the PMO molecule comprises from about 10 to about 28 nucleotides in length. In some aspects, the PMO molecule comprises from about 26 to about 28 nucleotides in length. In some aspects, the PMO molecule comprises a nucleic acid sequence having at least 90%, 95%, or 100% sequence identity any one of SEQ ID NOs; 100-169, or wherein the PMO molecule comprises a nucleic acid sequence having at least 20, 21, 22, 23, 24 nucleotides from a nucleic acid sequence selected from a group consisting of SEQ ID NOs; 100-169 with no more than one, two, three, or 4 mismatches, or consists of a nucleic acid sequence selected from a group consisting of SEQ ID NOs; 100-169. In some instances, the PMO molecule comprises a nucleic acid sequence selected from SEQ ID NOs; 144-169. In some instances, the PMO molecule comprises a nucleic acid sequence selected from SEQ ID NOs; 148-160. In some instances, the PMO molecule consists of a nucleic acid sequence selected from SEQ ID NOs; 144-169. In some instances, the PMO molecule consists of a nucleic acid sequence selected from SEQ ID NOs; 148-160.

In some aspects, the PMO molecule is delivered into a muscle cell. In some aspects, the PMO molecule hybridizes exon 50 of a pre-mRNA transcript of the DMD gene in said pre-mRNA transcript to generate a mRNA transcript encoding a truncated dystrophin protein. In some aspects, the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof. In some aspects, the PMO molecule is conjugated to the anti-transferrin receptor antibody or antigen binding fragment thereof via a linker. In some aspects, the linker is a cleavable linker. In some aspects, the linker is a non-cleavable linker. In some aspects, the linker is selected from the group consisting of a heterobifunctional linker, a homobifunctional linker, a linker comprising a maleimide group, a dipeptide moiety, a benzoic acid group or derivatives thereof, a C₁-C₆ alkyl group, and a combination thereof. In some aspects, the PMO conjugate has a PMO molecule to antibody ratio (DAR) of about 1:1, 2:1, 3:1, 4:1 5:1, 6:1, 7:1, 8:1, 9:1, 10:1, or higher. In some aspects, the PMO conjugate has a DAR of about 4.0. In some aspects, the PMO conjugate has a DAR of about 4.5. In some aspects, the PMO conjugate has a DAR of about 5.0. In some aspects, the PMO conjugate has a DAR of about 7.0. In some aspects, the PMO conjugate has a DAR of about 7.5. In some aspects, the PMO conjugate has a DAR of about 8.0. In some aspects, the PMO conjugate has a DAR of about 8.5. In some aspects, the PMO conjugate has a DAR of about 9.0. In some aspects, the PMO conjugate has a DAR of about 9.0. In some aspects, the PMO conjugate has a DAR of about 9.5. In some aspects, the PMO conjugate has a DAR of about 10.0. In some aspects, the PMO conjugate has a DAR of about 10.5. In some aspects, the PMO conjugate has a DAR of about 11.0. In some aspects, a composition comprises a plurality of PMO conjugates as described herein. In certain instances, the composition has a plurality of the PMO conjugates having an average DAR of about 1, 2, 3, 4, 5, 6, 7, 8, 9, 10, or higher. In some aspects, a plurality of the PMO conjugates has an average DAR in the range of 3.5-4.5. In some aspects, a plurality of the PMO conjugates has an average DAR in the range of 4-5. In some aspects, a plurality of the PMO conjugates has an average DAR in the range of 7-8. In some aspects, a plurality of the PMO conjugates has an average DAR in the range of 9.5-10.5. In some aspects, a plurality of the PMO conjugates has an average DAR of about 4. In some aspects, a plurality of the PMO conjugates has an average DAR of about 4.5. In some aspects, a plurality of the PMO conjugates has an average DAR of about 5.0. In some aspects, a plurality of the PMO conjugates has an average DAR of about 7.0. In some aspects, a plurality of the PMO conjugates has an average DAR of about 7.5. In some aspects, a plurality of the PMO conjugates has an average DAR of about 8.0. In some aspects, a plurality of the PMO conjugates has an average DAR of about 9.0. In some aspects, a plurality of the PMO conjugates has an average DAR of about 10.0. In some aspects, the PMO conjugate or the composition comprising a plurality of PMO conjugates is formulated for parenteral administration.

Also disclosed herein, in certain aspects, are methods of treating muscular dystrophy in a subject in need thereof comprising administering to said subject a phosphorodiamidate morpholino oligonucleotide (PMO) conjugate comprising an anti-transferrin receptor antibody or antigen binding fragment thereof conjugated to a PMO molecule, wherein the PMO molecule hybridizes to a site within an exon, an acceptor splice site, a donor splice site, or an exonic splice enhancer element of a pre-mRNA transcript of the DMD gene and induces exon 50 skipping in said pre-mRNA transcript to generate a mRNA transcript encoding a truncated dystrophin protein. In some aspects, the PMO molecule comprises a nucleic acid sequence having at least 90%, 95%, or 100% sequence identity any one of SEQ ID NOs; 100-169, or wherein the PMO molecule comprises a nucleic acid sequence having at least 20, 21, 22, 23, 24 nucleotides from a nucleic acid sequence selected from a group consisting of SEQ ID NOs; 100-169 with no more than one, two, three, or 4 mismatches, or consists of a nucleic acid sequence selected from a group consisting of SEQ ID NOs; 100-169. In some instances, the PMO molecule comprises a nucleic acid sequence selected from SEQ ID NOs; 144-169. In some instances, the PMO molecule comprises a nucleic acid sequence selected from SEQ ID NOs; 148-160. In some instances, the PMO molecule consists of a nucleic acid sequence selected from SEQ ID NOS: 144-169. In some instances, the PMO molecule consists of a nucleic acid sequence selected from SEQ ID NOs; 148-160. In some aspects, the PMO molecule is delivered into a muscle cell. In some aspects, the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof. In some aspects, the PMO molecule comprises from about 10 to about 30 nucleotides in length. In some aspects, the PMO molecule is conjugated to the anti-transferrin receptor antibody or antigen binding fragment thereof via a linker. In some aspects, the linker is a cleavable linker. In some instances, the linker is a non-cleavable linker. In some aspects, the linker is selected from the group consisting of a heterobifunctional linker, a homobifunctional linker, a linker comprising a maleimide group, a dipeptide moiety, a benzoic acid group or derivatives thereof, a C₁-C₆ alkyl group, and a combination thereof. In some aspects, a plurality of the PMO conjugates has an average of PMO molecule to antibody ratio (DAR) of about 1:1, 2:1, 3:1, 4:1, 5:1, 6:1, 7:1, 8:1, 9:1, 10:1, or higher. In some aspects, a plurality of the PMO conjugates has an average DAR in the range of 3.5-4.5. In some aspects, a plurality of the PMO conjugates has an average DAR in the range of 4-5. In some aspects, a plurality of the PMO conjugates has an average DAR in the range of 9.5-10.5. In some aspects, a plurality of the PMO conjugates has an average DAR in the range of 10.0-11.0. In some aspects, a plurality of the PMO conjugates has an average DAR of about 4.0 or 4.5. In some aspects, a plurality of the PMO conjugates has an average DAR of about 10.0 or 10.5. In some aspects, the PMO conjugate is administered parenterally. In some aspects, the truncated dystrophin proteins modulate muscular dystrophy. In some aspects, the muscular dystrophy is Duchenne muscular dystrophy or Becker muscular dystrophy.

Also disclosed herein, in certain aspects, are methods of inducing exon 50 skipping in a targeted pre-mRNA transcript of DMD gene, comprising: (a) contacting a muscle cell with a phosphorodiamidate morpholino oligonucleotide (PMO)-antibody conjugate, wherein the PMO-antibody conjugate comprises an anti-transferrin receptor antibody or antigen binding fragment thereof, and a PMO molecule targeting a site within an exon, an acceptor splice site, a donor splice site, or an exonic splice enhancer element of the targeted pre-mRNA transcript of the DMD gene; wherein the PMO molecule induces exon 50 skipping in the targeted pre-mRNA transcript, and wherein the PMO-antibody conjugate is preferentially delivered into the muscle cell: (b) hybridizing the PMO molecule to the targeted pre-mRNA transcript to induce exon 50 skipping in the targeted pre-mRNA transcript; and (c) translating a mRNA transcript produced from the targeted pre-mRNA transcript processed in step b) in the muscle cell to generate a truncated dystrophin protein. In some aspects, the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single-domain antibody (sdAb), or camelid antibody or antigen binding fragment thereof. In some aspects, the PMO molecule comprises from about 10 to about 30 nucleotides in length. In some aspects, the PMO molecule comprises a nucleic acid sequence having at least 90%, 95%, or 100% sequence identity any one of SEQ ID NOs; 100-169, or wherein the PMO molecule comprises a nucleic acid sequence having at least 20, 21, 22, 23, 24 nucleotides from a nucleic acid sequence selected from a group consisting of SEQ ID NOs; 100-169 with no more than one, two, three, or 4 mismatches, or consists of a nucleic acid sequence selected from a group consisting of SEQ ID NOs; 100-169. In some instances, the PMO molecule comprises a nucleic acid sequence selected from SEQ ID NOs; 144-169. In some instances, the PMO molecule comprises a nucleic acid sequence selected from SEQ ID NOs: 148-160. In some instances, the PMO molecule consists of a nucleic acid sequence selected from SEQ ID NOs; 144-169. In some instances, the PMO molecule consists of a nucleic acid sequence selected from SEQ ID NOs; 148-160. In some aspects, the PMO molecule targets exon 50. In some aspects, the PMO molecule is conjugated to the anti-transferrin receptor antibody or antigen binding fragment thereof via a linker. In some aspects, the linker is a cleavable linker. In some aspects, the linker is a non-cleavable linker. In some aspects, the linker is selected from the group consisting of a heterobifunctional linker, a homobifunctional linker, a linker comprising a maleimide group, a dipeptide moiety, a benzoic acid group or derivatives thereof, a C₁-C₆ alkyl group, and a combination thereof. In some aspects, a plurality of the PMO conjugates has an average of PMO to antibody ratio (DAR) of about 1:1, 2:1, 3:1, 4:1, 5:1, 6:1, 7:1, 8:1, 9:1, 10:1 or higher. In some aspects, the PMO conjugate has a DAR of about 1, 2, 3, 4, 5, 6, 7, 8, 9, 10 or higher. In some aspects, a plurality of the PMO conjugates has an average DAR in the range of 3.5-4.5. In some aspects, a plurality of the PMO conjugates has an average DAR in the range of 9.5-10.5. In some aspects, a plurality of the PMO conjugates has an average DAR of about 4. In some aspects, a plurality of the PMO conjugates has an average DAR of about 10. In some aspects, the method is an in vivo method.

Also disclosed herein, in certain aspects, are methods of inducing exon 50 skipping in a subject in need thereof comprising administering to said subject a phosphorodiamidate morpholino oligonucleotide (PMO) conjugate comprising an anti-transferrin receptor antibody or antigen binding fragment thereof conjugated to a PMO molecule comprising a nucleic acid sequence having at least 90%, 95%, or 100% sequence identity any one of SEQ ID NOs; 100-169, or comprising a nucleic acid sequence having at least 20, 21, 22, 23, 24 nucleotides from a nucleic acid sequence selected from a group consisting of SEQ ID NOs; 100-169 with no more than one, two, three, or 4 mismatches, or consisting of a nucleic acid sequence selected from a group consisting of SEQ ID NOs; 100-169; wherein the PMO molecule hybridizes to exon 50 of a pre-mRNA transcript of the DMD gene and induces exon 50 skipping in said pre-mRNA transcript to generate a mRNA transcript encoding a truncated dystrophin protein. In some aspects, the subject is affected by DMD.

Also disclosed herein, in certain aspects, are methods of restoring dystrophin in a subject in need thereof comprising administering to said subject a phosphorodiamidate morpholino oligonucleotide (PMO) conjugate comprising an anti-transferrin receptor antibody or antigen binding fragment thereof conjugated to a PMO molecule comprising a nucleic acid sequence having at least 90%, 95%, or 100% sequence identity any one of SEQ ID NOs; 100-169, or comprising a nucleic acid sequence having at least 20, 21, 22, 23, 24 nucleotides from a nucleic acid sequence selected from a group consisting of SEQ ID NOs; 100-169 with no more than one, two, three, or 4 mismatches, or consisting of a nucleic acid sequence selected from a group consisting of SEQ ID NOs; 100-169; wherein the PMO molecule hybridizes to exon 50 of a pre-mRNA transcript of the DMD gene and induces exon 50 skipping in said pre-mRNA transcript to generate a mRNA transcript encoding a truncated dystrophin protein. In some aspects, the subject is affected by DMD.

Also disclosed herein, in certain aspects, are methods of generating a truncated dystrophin protein in a subject in need thereof comprising administering to said subject a phosphorodiamidate morpholino oligonucleotide (PMO) conjugate comprising an anti-transferrin receptor antibody or antigen binding fragment thereof conjugated to a PMO molecule comprising a nucleic acid sequence having at least 90%, 95%, or 100% sequence identity any one of SEQ ID NOs; 100-169, or comprising a nucleic acid sequence having at least 20, 21, 22, 23, 24 nucleotides from a nucleic acid sequence selected from a group consisting of SEQ ID NOs; 100-169 with no more than one, two, three, or 4 mismatches, or consisting of a nucleic acid sequence selected from a group consisting of SEQ ID NOs; 100-169; wherein the PMO molecule hybridizes to exon 50 of a pre-mRNA transcript of the DMD gene and induces exon 50 skipping in said pre-mRNA transcript to generate a mRNA transcript encoding a truncated dystrophin protein. In some aspects, the subject is affected by DMD.

### BRIEF DESCRIPTION OF THE DRAWINGS

FIG. 1 is a bar graph showing the relative levels of exon 50 skipping activity in response to a library of 44 28-mer PMOs targeting exon 50 at a concentration of 30 μM in immortalized human skeletal muscle cells.

FIG. 2 is a bar graph showing the relative levels of exon 50 skipping activity in response to increasing concentrations (0.3 μM. I μM. 3 μM. 10 μM, and 30 μM) of 12 different 28-mer PMOs in immortalized human skeletal muscle cells.

FIG. 3 is a bar graph showing the relative levels of exon 50 skipping activity in response to PMOs with 4 different lengths including 22-mer. 24-mer. 26-mer, and 28-mer at concentrations of 10 μM and 30 μM in immortalized human skeletal muscle cells.

FIGS. 4A-C are plots illustrating the relative levels of exon 50 skipping activity in response to increasing concentrations of 3 different exon 50 skipping PMOs (hEx50)_Ac15_24mer, hEx50)_Ac17_24mer, or hEx50)_Ac77_24mer) in DMD patient-derived skeletal muscle cells. FIG. 4A is a line graph of the relative levels of exon 50 skipping activity in response to increasing concentrations (e.g., 0.3 μM. I μM. 3 μM. 10 μM. 30 μM. 50 μM) of 3 PMOs skipping exon 50 of DMD in human skeletal muscle cells with deletion of exon 51 (del Ex51). FIG. 4B is a line graph of the relative levels of dystrophin restoration in response to increasing concentrations of 3 PMOs skipping exon 50 of DMD in human skeletal muscle cells with deletion of exon 51 (del Ex51). FIG. 4C is a line graph showing the correlation between the levels of exon 50 skipping activities and the levels of dystrophin restoration of 3 PMOs skipping exon 50 of DMD in the human skeletal muscle cells with deletion of exon 51 in the DMD gene.

FIGS. 5A-C are bar graphs illustrating the relative levels of exon 50 skipping activity of the hEx50)_Ac17_24 at concentrations of 3 μM, 10 μM, and 30 μM in human primary myotubes from healthy donors and DMD patients with DMD exon 51 deletion (del51) and relative protein levels of dystrophin restoration of hEx50_Ac17_24 at the concentrations of 3 μM, 10 μM, and 30 μM in human primary myotubes from DMD patients with DMD deletion of exon 51 (del51). FIG. 5A is a bar graph showing the relative levels of exon 50 skipping activity in response to hEx50)_Ac17_24 at the concentration of 3 μM, 10 μM, or 30 μM in primary myotubes from 3 different healthy donors (MB07. MB09, and WO18). FIG. 5B is a bar graph of the relative levels of exon 50 skipping activity in response to hEx50_Ac17_24 at the concentrations of 3 μM or 10 μM, and 30 μM in DMD primary myotubes derived from 2 different DMD patients having deletion of DMD exon 51 (DMD primary #1 del.51, primary #2 del. 51). FIG. 5C is a bar graph of the relative protein levels of dystrophin restoration of the hEx50_Ac17_24 at the concentrations of 3 μM, 10 UM or 30 μM in human primary myotubes from DMD patients having deletion of exon 51 (DMD primary #1 del.51, primary #2 del. 51).

FIGS. 6A-C depict levels of dystrophin protein expression in response to the hEx50)_Ac17_24 or hEx50_Ac77_24 PMOs in human primary myotubes derived from DMD patients. FIG. 6A shows images of immunofluorescence staining of dystrophin positive fibers in non-treated human primary myotubes from healthy donor and DMD primary myotubes transfected with hEx50_Ac17_24. FIGS. 6B-C are plots for the concentration response curve of the relative levels of immunofluorescence staining of dystrophin in response to increasing concentrations (1 μM, 3 μM, 10 μM, and 30 μM) of hEx50_Ac17_24 or hEx50_Ac77_24 in the DMD patient cells with deletion of exon 51 of DMD.

FIG. 7 is a bar graph illustrating the hEx50_Ac17_24 (PMO50)) concentrations in skeletal and cardiac muscle tissues (gastrocnemius, quadricep, diaphragm, and heart) obtained from humanized DMD transgenic mice at day 14 that have been administered a single intravenous (IV) IV bolus injection of DAR 4 hEx50_Ac17_24 AOC (PMO50) AOC or DAR 10 hEx50_Ac17_24 (PMO50) AOC at the PMO dose of 30 mg/kg at day 0).

FIG. 8 is a bar graph illustrating the relative levels of exon 50 skipping activities in muscle tissues obtained from humanized DMD transgenic mice at day 14 that have been administered a single IV bolus injection of DAR 4 hEx50_Ac17_24 (PMO50)) AOC or DAR 10 hEx50_Ac17_24 (PMO50) AOC at a dose of 30 mg/kg at day ( )

### DETAILED DESCRIPTION OF THE DISCLOSURE

Disclosed herein, in some aspects, are antibody-polynucleic acid (oligonucleotide) conjugate (AOC) compositions for the treatment of muscle dystrophy. Also disclosed herein, in some aspects, are methods of treating muscle dystrophy caused by an incorrectly spliced DMD mRNA transcript in a subject in need thereof, the method comprising: administering to the subject an antibody-polynucleic acid conjugate; wherein the antibody-polynucleic acid conjugate induces alteration in the incorrectly spliced pre-mRNA dystrophy transcript to induce exon 50 skipping of the DMD mRNA transcript to generate a fully processed DMD mRNA transcript; and wherein the fully processed DMD mRNA transcript encodes a functional and truncated dystrophin protein, thereby treating the disease or disorder in the subject. As used herein, the term “polynucleic acid” is interchangeably used with the term “oligonucleotide”.

Disclosed herein, in some aspects, are antibody-antisense oligonucleotide (ASO) conjugate or antibody-phosphorodiamidate morpholino oligomer (PMO) conjugate compositions for the treatment of muscle dystrophy. Also disclosed herein are methods of treating muscle dystrophy caused by an incorrectly spliced DMD mRNA transcript in a subject in need thereof, the method comprising: administering to the subject an antibody-ASO conjugate or an antibody-PMO conjugate; wherein the ASO or PMO induces alteration in the incorrectly spliced pre-mRNA dystrophy transcript to induce exon 50 skipping of the DMD mRNA transcript to generate a fully processed DMD mRNA transcript; and wherein the fully processed DMD mRNA transcript encodes a functional and truncated dystrophin protein, thereby treating the disease or disorder in the subject.

In some instances, one such area where antibody-polynucleic acid conjugate is used is for treating muscular dystrophy. Muscular dystrophy encompasses several diseases that affect the muscle. Duchenne muscular dystrophy is a severe form of muscular dystrophy and caused by mutations in the DMD gene. In some instances, mutations in the DMD gene disrupt the translational reading frame and results in non-functional dystrophin protein.

Described herein, in certain aspects, are methods and compositions relating to nucleic acid-based therapy to induce an insertion, deletion, duplication, or alteration in an incorrectly spliced mRNA transcript to induce exon skipping or exon inclusion, which is used to restore the translational reading frame. In some aspects, also described herein include methods and compositions for treating a disease or disorder characterized by an incorrectly processed mRNA transcript, in which after removal of an exon, the mRNA is capable of encoding a functional protein, thereby treating the disease or disorder. In additional aspects, described herein include pharmaceutical compositions and kits for treating the same.

### RNA Processing

RNA has a central role in regulation of gene expression and cell physiology. Proper processing of RNA is important for the translation of functional proteins. Alterations in RNA processing such as a result of incorrect splicing of RNA can result in disease. For example, mutations in a splice site causes exposure of a premature stop codon, a loss of an exon, or inclusion of an intron. In some instances, alterations in RNA processing results in an insertion, deletion, or duplication. In some instances, alterations in RNA processing results in an insertion, deletion, or duplication of an exon. Alterations in RNA processing, in some cases, results in an insertion, deletion, or duplication of an intron.

### Exon Skipping

As used herein, the term “pre-mRNA” refers to the product of transcription which is comprised of both exons (coding sequences) and introns (non-coding sequences). Exon skipping is a form of RNA splicing. In some cases, exon skipping occurs when an exon is skipped over the pre-mRNA transcript or is spliced out of the processed mRNA. As a result of exon skipping, the processed mRNA does not contain the skipped exon. In some instances, exon skipping results in expression of an altered transcript and/or mRNA product. For instance, exon 50) skipping occurs when exon 50 is skipped over in the pre-mRNA transcript or is spliced out of the processed DMD mRNA. As a result of the exon 50 skipping, the processed DMD mRNA does not contain the skipped exon 50. In some instances, exon 50 skipping results in the expression of a truncated dystrophin protein. In some instances, exon 50 skipping results in the expression of a functional dystrophin protein. In some instances, exon 50 skipping results in the expression of a truncated and functional dystrophin protein.

In some instances, morpholino or phosphorodiamidate morpholino oligonucleotide (PMO)-antibody conjugates (PMO-AOC) are used to induce exon skipping. In some instances, morpholino or phosphorodiamidate morpholino oligonucleotide (PMO)-antibody conjugates are used to deliver PMOs for inducing exon skipping (e.g., in a cell, preferably in a muscle cell, etc.). In some instances, the delivered PMOs are used to induce exon skipping. For example, the PMOs bind splice sites or exonic enhancers. In some instances, binding of PMOs to specific mRNA or pre-mRNA sequences generates double-stranded regions within the specific mRNA or pre-mRNA sequences. In some instances. PMOs bind to acceptor or donor splice site at the beginning and/or at the end of an exon. In some instances. PMOs bind to a site within an exon. In some instances, morpholino or phosphorodiamidate morpholino oligonucleotide (PMO)-antibody conjugates are used to induce exon 50 skipping. In some instances, morpholino or phosphorodiamidate morpholino oligonucleotide (PMO)-antibody conjugates are used to deliver PMOs for inducing exon 50 skipping. The delivered PMOs are used to induce exon 50 skipping. For example, the delivered PMOs bind to at least one of splice sites or exonic enhancers of exon 50. In some instances, binding of PMOs to specific mRNA or pre-mRNA sequences generates double-stranded regions within the specific mRNA or pre-mRNA sequences. In some instances. PMOs bind to acceptor or donor splice site at the beginning and/or at the end of exon 50. In some instances. PMOs bind to acceptor splice site at the beginning (e.g., 5′-end)/end (e.g., 3′-end) of exon 50. In some instances. PMOs bind to donor splice site at the beginning (e.g., 5′-end)/end (e.g., 3′-end) of exon 50. In some instances. PMOs bind to a site within exon 50. In some instances, antisense oligonucleotides (AONs. ASOs) are used to induce exon skipping. As used herein, the term “AONs” is interchangeably used with the term “ASOs” and both refer to antisense oligonucleotides. In some instances, AONs are short nucleic acid sequences that bind to specific mRNA or pre-mRNA sequences. For example, AONs bind to splice sites or exonic enhancers. In some instances, binding of AONs to specific mRNA or pre-mRNA sequences generates double-stranded regions. In some instances, formation of double-stranded regions occurs at sites where the spliceosome or proteins associated with the spliceosome would normally bind to and causes exons to be skipped. In some instances, skipping of exons results in restoration of the transcript reading frame and allows for production of an at least partially functional dystrophin protein.

### Indications

In some aspects, a polynucleic acid molecule (oligonucleotide, e.g., PMO, ASO, etc.) or a pharmaceutical composition comprising the polynucleic acid molecule described herein is used for the treatment of a disease or disorder characterized with a defective mRNA. In some aspects, a polynucleic acid molecule (oligonucleotide, e.g., PMO, ASO, etc.) or a pharmaceutical composition comprising the polynucleic acid molecule described herein is used for the treatment of disease or disorder by inducing an insertion, deletion, duplication, or alteration in an incorrectly spliced mRNA transcript to induce exon skipping or exon inclusion.

A large percentage of human protein-coding genes are alternatively spliced. In some instances, a mutation results in improperly spliced or partially spliced mRNA. For example, a mutation can be in at least one of a splice site in a protein coding gene, a silencer or enhancer sequence, exonic sequences, or intronic sequences. In some instances, a mutation results in gene dysfunction. In some instances, a mutation results in a disease or disorder.

Improperly spliced or partially spliced mRNA in some instances causes a neuromuscular disease or disorder. Exemplary neuromuscular diseases include muscular dystrophy such as Duchenne muscular dystrophy, Becker muscular dystrophy, facioscapulohumeral muscular dystrophy, congenital muscular dystrophy, or myotonic dystrophy. In some instances, muscular dystrophy is genetic. In some instances, muscular dystrophy is caused by a spontaneous mutation. Becker muscular dystrophy and Duchenne muscular dystrophy have been shown to involve mutations in the DMD gene, which encodes the protein dystrophin.

In some instances, improperly spliced or partially spliced mRNA causes Duchenne muscular dystrophy. Duchenne muscular dystrophy results in severe muscle weakness and is caused by mutations in the DMD gene that abolishes the production of functional dystrophin. In some instances. Duchenne muscular dystrophy is a result of a mutation in exon 50 in the DMD gene. In some instances, multiple exons are mutated/deleted. For example, mutations of exons 45, 51, and 53 are common in Duchenne muscular dystrophy patients. In some instances. Duchenne muscular dystrophy is a result of mutation of exon 50. In some instances. Duchenne muscular dystrophy is a result of deletion of exon 50.

In some instances, a polynucleic acid-antibody conjugate or a pharmaceutical composition comprising the polynucleic acid-antibody conjugate as described herein is used for the treatment of muscular dystrophy. In some instances, a polynucleic acid-antibody conjugate or a pharmaceutical composition comprising the polynucleic acid-antibody conjugate as described herein is used for the treatment of Duchenne muscular dystrophy. Becker muscular dystrophy, facioscapulohumeral muscular dystrophy, congenital muscular dystrophy, or myotonic dystrophy. In some instances, a polynucleic acid-antibody conjugate or a pharmaceutical composition comprising the polynucleic acid-antibody conjugate as described herein is used for the treatment of Duchenne muscular dystrophy. In some instances, a PMO-antibody conjugate or a pharmaceutical composition comprising the PMO-antibody conjugate as described herein is used to induce exon 50 skipping for the treatment of muscular dystrophy. In some instances, a PMO-antibody conjugate or a pharmaceutical composition comprising the PMO-antibody conjugate as described herein is used to induce exon 50 skipping for the treatment of Duchenne muscular dystrophy or Becker muscular dystrophy. In some instances, a PMO-antibody conjugate or a pharmaceutical composition comprising the PMO-antibody conjugate as described herein is used to induce exon 50 skipping for the treatment of Duchenne muscular dystrophy.

### Antibody-Polynucleic Acid Conjugate

In some aspects, the antibody is conjugated to a polynucleic acid molecule. The polynucleic acid molecule can be ASO or PMO. In some instances, the polynucleic acid molecule is PMO. In some instances, the antibody is an anti-transferrin receptor (anti-CD71) antibody or antigen binding fragment thereof. In some aspects, the antibody is conjugated to a polynucleic acid molecule non-specifically. In some instances, the antibody is conjugated to a polynucleic acid molecule via a lysine residue. In some instances, the antibody is conjugated to a polynucleic acid molecule via a cysteine residue. In some instances, the antibody is conjugated to a polynucleic acid molecule via a lysine residue or a cysteine residue, in a non-site specific manner. In some instances, the antibody is conjugated to a polynucleic acid molecule via a lysine residue (e.g., lysine residue present in the antibody in a non-site specific manner. In some cases, the antibody is conjugated to a polynucleic acid molecule via a cysteine residue (e.g., cysteine residue present in the antibody in a non-site specific manner.

In some aspects, the antibody is conjugated to a polynucleic acid molecule in a site-specific manner. In some instances, the antibody is conjugated to a polynucleic acid molecule through a lysine residue, a cysteine residue, at the 5′-terminus, at the 3′-terminus, an unnatural amino acid, or an enzyme-modified or enzyme-catalyzed residue, via a site-specific manner. In some instances, the antibody is conjugated to a polynucleic acid molecule through a lysine residue (e.g., lysine residue present in the antibody via a site-specific manner). In some instances, the antibody is conjugated to a polynucleic acid molecule through a cysteine residue (e.g., cysteine residue present in the antibody via a site-specific manner). In some instances, the antibody is conjugated to a polynucleic acid molecule at the 5′-terminus via a site-specific manner. In some instances, the antibody is conjugated to a polynucleic acid molecule at the 3′-terminus via a site-specific manner. In some instances, the antibody is conjugated to a polynucleic acid molecule through an unnatural amino acid via a site-specific manner. In some instances, the antibody is conjugated to a polynucleic acid molecule through an enzyme-modified or enzyme-catalyzed residue via a site-specific manner. In some instances, the antibody is conjugated to a polynucleic acid molecule via a linker or one or more linkers.

In some aspects, one or more polynucleic acid molecules are conjugated to an antibody. The one or more polynucleic acid molecules can be ASOs or PMOs. In some instances, the one or more polynucleic acid molecules are PMOs. The antibody can be an anti-transferrin receptor (anti-CD71) antibody or antigen binding fragment thereof. In some instances, about 1, 2, 3, 4, 5, 6, 7, 8, 9, 10, 11, 12, 13, 14, 15, 16, or more polynucleic acid molecules are conjugated to one antibody. In some instances, about 1 polynucleic acid molecule is conjugated to one antibody. In some instances, about 2 polynucleic acid molecules are conjugated to one antibody. In some instances, about 3 polynucleic acid molecules are conjugated to one antibody. In some instances, about 4 polynucleic acid molecules are conjugated to one antibody. In some instances, about 5 polynucleic acid molecules are conjugated to one antibody. In some instances, about 6 polynucleic acid molecules are conjugated to one antibody. In some instances, about 7 polynucleic acid molecules are conjugated to one antibody. In some instances, about 8 polynucleic acid molecules are conjugated to one antibody. In some instances, about 9 polynucleic acid molecules are conjugated to one antibody. In some instances, about 10 polynucleic acid molecules are conjugated to one antibody. In some instances, about 11 polynucleic acid molecules are conjugated to one antibody. In some instances, about 12 polynucleic acid molecules are conjugated to one antibody. In some instances, about 13 polynucleic acid molecules are conjugated to one antibody. In some instances, about 14 polynucleic acid molecules are conjugated to one antibody. In some instances, about 15 polynucleic acid molecules are conjugated to one antibody. In some instances, about 16 polynucleic acid molecules are conjugated to one antibody. In some cases, the one or more polynucleic acid molecules are the same. In other cases, the one or more polynucleic acid molecules are different.

In some aspects, the number of polynucleic acid molecule conjugated to an antibody forms a ratio. In some instances, the ratio is referred to as a DAR (drug-to-antibody ratio), in which the drug as referred to herein is the polynucleic acid molecule. In some instances, the DAR of the polynucleic acid molecule to antibody is about 1, 2, 3, 4, 5, 6, 7, 8, 9, 10, 11, 12, 13, 14, 15, 16, or greater. In some instances, the DAR of the polynucleic acid molecule to antibody is approximately 1, 2, 3, 4, 5, 6, 7, 8, 9, 10, 11, 12, 13, 14, 15, 16, or greater. In some instances, the DAR includes whole number as well as fractions or decimal of a DAR. For instance, the fractions or decimal of a DAR includes X.1. X.2. X.3. X.4. X.5. X.6. X.7. X.8. X.9 (e.g., 2.1, 2.2, 2.3, 2.4, 2.5, 2.6, 2.7, 2.8, 2.9, etc.). In some instances, the DAR of the polynucleic acid molecule to antibody is about 1 or greater. In some instances, the DAR of the polynucleic acid molecule to antibody is about 2 or greater. In some instances, the DAR of the polynucleic acid molecule to antibody is about 3 or greater. In some instances, the DAR of the polynucleic acid molecule to antibody is about 4 or greater. In some instances, the DAR of the polynucleic acid molecule to antibody is about 5 or greater. In some instances, the DAR of the polynucleic acid molecule to antibody is about 6 or greater. In some instances, the DAR of the polynucleic acid molecule to antibody is about 7 or greater. In some instances, the DAR of the polynucleic acid molecule to antibody is about 8 or greater. In some instances, the DAR of the polynucleic acid molecule to antibody is about 9 or greater. In some instances, the DAR of the polynucleic acid molecule to antibody is about 10 or greater. In some instances, the DAR of the polynucleic acid molecule to antibody is about 11 or greater. In some instances, the DAR of the polynucleic acid molecule to antibody is about 12 or greater.

In some instances, the DAR of the polynucleic acid molecule to antibody is about 1, 2, 3, 4, 5, 6, 7, 8, 9, 10, 11, 12, 13, 14, 15, or 16. In some instances, the DAR of the polynucleic acid molecule to antibody is about 1. In some instances, the DAR of the polynucleic acid molecule to antibody is about 2. In some instances, the DAR of the polynucleic acid molecule to antibody is about 3. In some instances, the DAR of the polynucleic acid molecule to antibody is about 4. In some instances, the DAR of the polynucleic acid molecule to antibody is about 5. In some instances, the DAR of the polynucleic acid molecule to antibody is about 6. In some instances, the DAR of the polynucleic acid molecule to antibody is about 7. In some instances, the DAR of the polynucleic acid molecule to antibody is about 8. In some instances, the DAR of the polynucleic acid molecule to antibody is about 9. In some instances, the DAR of the polynucleic acid molecule to antibody is about 10. In some instances, the DAR of the polynucleic acid molecule to antibody is about 11. In some instances, the DAR of the polynucleic acid molecule to antibody is about 12. In some instances, the DAR of the polynucleic acid molecule to antibody is about 13. In some instances, the DAR of the polynucleic acid molecule to antibody is about 14. In some instances, the DAR of the polynucleic acid molecule to antibody is about 15. In some instances, the DAR of the polynucleic acid molecule to antibody is about 16.

In some instances, the DAR of the polynucleic acid molecule to antibody is 1, 2, 3. 4, 5, 6, 7, 8, 9, 10, 11, 12, 13, 14, 15, or 16. In some instances, the DAR of the polynucleic acid molecule to antibody is 1. In some instances, the DAR of the polynucleic acid molecule to antibody is 2. In some instances, the DAR of the polynucleic acid molecule to antibody is 4. In some instances, the DAR of the polynucleic acid molecule to antibody is 6. In some instances, the DAR of the polynucleic acid molecule to antibody is 8. In some instances, the DAR of the polynucleic acid molecule to antibody is 12. In some instances, the DAR of the polynucleic acid molecule to antibody is 16.

In some aspects, a composition comprises a plurality of antibody-polynucleic acid conjugates. In some instances, the number of polynucleic acid molecule conjugated to an antibody forms a ratio. In some instances, the ratio is referred to as a DAR (drug-to-antibody ratio), in which the drug as referred to herein is the polynucleic acid molecule. In some instances, the plurality of antibody-polynucleic acid conjugates in the composition has the same DAR. In some instances, the plurality of antibody-polynucleic acid conjugates in the composition has different DARs. In some instances, at least two of the antibody-polynucleic acid conjugates in the composition have different DARs to each other. In some instances, the DAR is an average DAR (drug-to-antibody ratio), which is an average number of the DARs of the plurality of antibody-polynucleic acid conjugates in the composition. In some instances, the average DAR of the polynucleic acid molecule to antibody is about 1, 2, 3, 4, 5, 6, 7, 8, 9, 10, 11, 12, 13, 14, 15, 16, or greater. In some instances, the average DAR of the polynucleic acid molecule to antibody is approximately 1, 2, 3, 4, 5, 6, 7, 8, 9, 10, 11, 12, 13, 14, 15, 16, or greater. In some instances, the average DAR includes whole number as well as fractions or decimal of a DAR. In some instances, the average DAR of the polynucleic acid molecule to antibody is about 1 or greater. In some instances, the average DAR of the polynucleic acid molecule to antibody is about 2 or greater. In some instances, the average DAR of the polynucleic acid molecule to antibody is about 3 or greater. In some instances, the average DAR of the polynucleic acid molecule to antibody is about 4 or greater. In some instances, the average DAR of the polynucleic acid molecule to antibody is about 5 or greater. In some instances, the average DAR of the polynucleic acid molecule to antibody is about 6 or greater. In some instances, the average DAR of the polynucleic acid molecule to antibody is about 7 or greater. In some instances, the average DAR of the polynucleic acid molecule to antibody is about 8 or greater. In some instances, the average DAR of the polynucleic acid molecule to antibody is about 9 or greater. In some instances, the average DAR of the polynucleic acid molecule to antibody is about 10 or greater. In some instances, the average DAR of the polynucleic acid molecule to antibody is about 11 or greater. In some instances, the average DAR of the polynucleic acid molecule to antibody is about 12 or greater.

In some instances, the average DAR of the polynucleic acid molecule to antibody is about 1, 2, 3, 4, 5, 6, 7, 8, 9, 10, 11, 12, 13, 14, 15, or 16. In some instances, the average DAR of the polynucleic acid molecule to antibody is about 1. In some instances, the average DAR of the polynucleic acid molecule to antibody is about 2. In some instances, the average DAR of the polynucleic acid molecule to antibody is about 3. In some instances, the average DAR of the polynucleic acid molecule to antibody is about 4. In some instances, the average DAR of the polynucleic acid molecule to antibody is about 5. In some instances, the average DAR of the polynucleic acid molecule to antibody is about 6. In some instances, the average DAR of the polynucleic acid molecule to antibody is about 7. In some instances, the average DAR of the polynucleic acid molecule to antibody is about 8. In some instances, the average DAR of the polynucleic acid molecule to antibody is about 9. In some instances, the average DAR of the polynucleic acid molecule to antibody is about 10. In some instances, the average DAR of the polynucleic acid molecule to antibody is about 11. In some instances, the average DAR of the polynucleic acid molecule to antibody is about 12. In some instances, the average DAR of the polynucleic acid molecule to antibody is about 13. In some instances, the average DAR of the polynucleic acid molecule to antibody is about 14. In some instances, the DAR of the polynucleic acid molecule to antibody is about 15. In some instances, the average DAR of the polynucleic acid molecule to antibody is about 16.

In some instances, the average DAR of the polynucleic acid molecule to antibody is 1, 2, 3, 4, 5, 6, 7, 8, 9, 10, 11, 12, 13, 14, 15, or 16. In some instances, the average DAR of the polynucleic acid molecule to antibody is 1. In some instances, the average DAR of the polynucleic acid molecule to antibody is 2. In some instances, the average DAR of the polynucleic acid molecule to antibody is 4. In some instances, the average DAR of the polynucleic acid molecule to antibody is 6. In some instances, the average DAR of the polynucleic acid molecule to antibody is 8. In some instances, the average DAR of the polynucleic acid molecule to antibody is 12. In some instances, the average DAR of the polynucleic acid molecule to antibody is 16.

In some instances, the average DAR of the polynucleic acid molecule to antibody is in the range of 1.5-2.5, 2.5-3.5, 3.5-4.5, 4.5-5.5, 5.5-6.5, 6.5-7.5, 7.5-8.5, 8.5-9.5, 9.5-10.5, 10.5-11.5, 11.5-12.5, 12.5-13.5. 13.5-14.5. 14.5-15.5. 15.5-16.5, or 16.5-17.5. In some instances, the average DAR of the polynucleic acid molecule to antibody is in the range of 1.5-2.5. In some instances, the average DAR of the polynucleic acid molecule to antibody is in the range of 2.5-3.5. In some instances, the average DAR of the polynucleic acid molecule to antibody is in the range of 3.5-4.5. In some instances, the average DAR of the polynucleic acid molecule to antibody is in the range of 4.5-5.5. In some instances, the average DAR of the polynucleic acid molecule to antibody is in the range of 5.5-6.5. In some instances, the average DAR of the polynucleic acid molecule to antibody is in the range of 6.5-7.5. In some instances, the average DAR of the polynucleic acid molecule to antibody is in the range of 7.5-8.5. In some instances, the average DAR of the polynucleic acid molecule to antibody is in the range of 8.5-9.5. In some instances, the average DAR of the polynucleic acid molecule to antibody is in the range of 9.5-10.5. In some instances, the average DAR of the polynucleic acid molecule to antibody is in the range of 10.5-11.5. In some instances, the average DAR of the polynucleic acid molecule to antibody is in the range of 11.5-12.5. In some instances, the average DAR of the polynucleic acid molecule to antibody is in the range of 12.5-13.5. In some instances, the average DAR of the polynucleic acid molecule to antibody is in the range of 13.5-14.5. In some instances, the average DAR of the polynucleic acid molecule to antibody is in the range of 14.5-15.5. In some instances, the DAR of the polynucleic acid molecule to antibody is in the range of 15.5-16.5. In some instances, the average DAR of the polynucleic acid molecule to antibody is in the range of 16.5-17.5.

In some instances, a conjugate comprising a polynucleic acid molecule and an antibody has improved activity as compared to a conjugate comprising a polynucleic acid molecule without an antibody. In some instances, improved activity results in enhanced biologically relevant functions, e.g., improved stability, affinity, binding, functional activity, and efficacy in treatment or prevention of a disease state. In some instances, the disease state is a result of one or more mutated exons of a gene. In some instances, the conjugate comprising a polynucleic acid molecule and an antibody results in increased exon skipping of the one or more mutated exons as compared to the conjugate comprising a polynucleic acid molecule without an antibody. In some instances, exon skipping is increased by at least or about 5%, 10%, 20%, 25%, 30%, 40%, 50%, 60%, 70%, 80%, 90%, 95%, or more than 95% in the conjugate comprising polynucleic acid molecule and antibody as compared to the conjugate comprising polynucleic acid molecule without an antibody.

### Pmo Molecule of the Pmo-Antibody Conjugate

In some aspects, the polynucleic acid is an antisense oligonucleotide (ASO) or a PMO molecule. In some aspects, the antibody-polynucleic acid conjugate is an ASO-antibody conjugate. In some aspects, the antibody-polynucleic acid conjugate is a PMO-antibody conjugate. In some aspects, a PMO molecule of the PMO-antibody conjugate described herein induces exon 50 skipping to induce an alteration in an incorrectly spliced mRNA transcript. In some instances, the PMO molecule restores the translational reading frame of the dystrophin protein by altering the incorrectly spliced mRNA transcript. In some instances, the PMO molecule results in a functional and truncated dystrophin protein by restoring the translational reading frame of the dystrophin protein.

In some aspects, a polynucleic acid molecule is conjugated to an antibody for delivery to a site of interest. In some cases, a PMO molecule is conjugated to an antibody. In some cases, a PMO molecule is conjugated to an antibody for delivery to a site of interest.

In some aspects, a PMO molecule is conjugated to an antibody for delivery to a muscle cell. In some cases, a PMO molecule for skipping exon 50 is conjugated to an antibody. In some cases, a PMO molecule for skipping exon 50 is conjugated to an antibody for delivery to a muscle cell.

In some instances, an antibody is conjugated to at least one PMO molecule. In some instances, the antibody is conjugated to the at least one PMO molecule to form an PMO-antibody conjugate. In some aspects, the antibody is conjugated to the 5′ terminus of the PMO molecule, the 3′ terminus of the PMO molecule, an internal site on the PMO molecule, or in any combinations thereof. In some instances, the antibody is conjugated to at least two PMO molecules. In some instances, the antibody is conjugated to at least 2, 3, 4, 5, 6, 7, 8, 9, 10 or more PMO molecules.

In some instances, a PMO molecule of the PMO-antibody conjugate targets and hybridizes to a pre-mRNA sequence of the DMD gene. In some instances, the PMO molecule targets and hybridizes a splice site of exon 50 of the pre-mRNA sequence of the DMD gene. In some instances, the PMO molecule targets and hybridizes a cis-regulatory element of exon 50 of the pre-mRNA sequence of the DMD gene. In some instances, the PMO molecule targets and hybridizes a trans-regulatory element of exon of the pre-mRNA sequence of the DMD gene. In some instances, the PMO molecule targets exonic splice enhancers or intronic splice enhancers of exon 50 of the pre-mRNA sequence of the DMD gene. In some instances, the PMO molecule targets and hybridizes exonic splice silencers or intronic splice silencers of exon 50 of the pre-mRNA sequence of the DMD gene. In some instances, the PMO molecule targets and hybridizes to the acceptor site of exon 50 of the pre-mRNA sequence of the DMD gene. In some instances, the PMO molecule targets and hybridizes to exon 50 of the pre-mRNA sequence of the DMD gene.

In some instances, a PMO molecule of the PMO-antibody conjugate targets and hybridizes a sequence within introns or exons of the pre-mRNA sequence of the DMD gene. For example, the PMO molecule targets and hybridizes to a sequence in exon 50 of the pre-mRNA sequence of the DMD gene that mediates splicing of said exon. In some instances, the PMO molecule targets an exon recognition sequence of the pre-mRNA sequence of the DMD gene. In some instances, the PMO molecule targets a sequence upstream of an exon of the pre-mRNA sequence of the DMD gene. In some instances, the PMO molecule targets a sequence downstream of an exon of the pre-mRNA sequence of the DMD gene.

As described above, a PMO molecule targets an incorrectly processed mRNA transcript which results in a neuromuscular disease or disorder. In some cases, a neuromuscular disease or disorder is Duchenne muscular dystrophy or Becker muscular dystrophy.

In some instances, the polynucleic acid molecule (e.g., a PMO molecule, an antisense oligonucleotide, etc.) targets a region (a sequence) adjacent to a mutated exon. In another instance, if there is a mutation in exon 50, the polynucleic acid molecule targets a sequence in exon 50 (e.g., a region within exon 50) of the pre-mRNA sequence of the DMD gene so that exon 50 is skipped.

In some cases, a polynucleic acid molecule described herein targets a region that is at the exon-intron junction of exon 50 of the pre-mRNA sequence of the DMD gene. In some cases, a polynucleic acid molecule described herein targets a region that is at the exon-intron junction of exon 50 of the pre-mRNA sequence of the DMD gene.

In some instances, the PMO molecule of the PMO-antibody conjugate hybridizes to a target region that is at either the 5′ intron-exon junction or the 3′ exon-intron junction of exon 50 of the pre-mRNA of the DMD gene.

In some cases, the polynucleic acid molecule hybridizes to a target region that is at the 5′ intron-exon junction of exon 50 of the pre-mRNA of the DMD gene.

In some cases, the PMO molecule hybridizes to a target region that is at the 3′ exon-intron junction of exon 50 of the pre-mRNA of the DMD gene.

In some instances, a PMO molecule of the PMO-antibody conjugate described herein targets a splice site of exon 50 of the pre-mRNA of the DMD gene. In some cases, a PMO molecule of the PMO-antibody conjugate described herein targets a splice site of exon 50 of the pre-mRNA of the DMD gene. As used herein, a splice site includes a canonical splice site, a cryptic splice site or an alternative splice site that is capable of inducing an insertion, deletion, duplication, or alteration in an incorrectly spliced mRNA transcript to induce exon 50 skipping.

In some instances, the PMO molecule of the PMO-antibody conjugate hybridizes to a target region that is proximal to the exon-intron junction. In some instances, a PMO molecule described herein targets a region at least 1000 nucleotides (nt), 500 nt. 400 nt. 300 nt. 200 nt. 100 nt. 80 nt. 60 nt. 50 nt. 40 nt. 30 nt. 20 nt. 10 nt, or 5 nt upstream (or the 5′) of exon 50 of the pre-mRNA of the DMD gene.

In some instances, the PMO molecule of the PMO-antibody conjugate hybridizes to a target region that is downstream (or 3″) to exon 50 of the pre-mRNA of the DMD gene. In some instances, the polynucleic acid molecule hybridizes to a target region that is about 5, 10, 15, 20, 50, 100, 200, 300, 400 or 500 nt downstream (or 3″) to exon 50 of the pre-mRNA of the DMD gene.

In some instances, a PMO molecule of the PMO-antibody conjugate described herein targets an internal region within exon 50 of the pre-mRNA of the DMD gene.

In some aspects, the PMO molecule of the PMO-antibody conjugate described herein targets a partially spliced mRNA sequence comprising exon 50 of the pre-mRNA of the DMD gene. In some instances, the PMO molecule hybridizes to a target region that is upstream (or 5′) to exon 50 of the partially spliced mRNA of the DMD gene. In some instances, the PMO molecule hybridizes to a target region that is about 5, 10, 15, 20, 50, 100, 200, 300, 400 or 500 bp upstream (or 5′) to exon 50 of the partially spliced mRNA of the DMD gene. In some instances, the PMO molecule hybridizes to a target region that is downstream (or 3″) to exon 50 of the partially spliced mRNA of the DMD gene. In some instances, the PMO molecule hybridizes to a target region that is about 5, 10, 15, 20, 50, 100, 200, 300, 400 or 500 bp downstream (or 3″) to exon 50 of the pre-mRNA of the DMD gene.

In some instances, the PMO molecule hybridizes to a target region that is within exon 50 of the pre-mRNA of the DMD gene. In some instances, the PMO molecule hybridizes to a target region that is at either the 5′ intron-exon 50 junction or the 3′ exon 50-intron junction of the pre-mRNA of the DMD gene. In some instances, the PMO molecule hybridizes to a target region that is within the acceptor site of exon 50 of the pre-mRNA of the DMD gene. In some instances, the PMO molecule hybridizes to a target region that is at either the 5′ intron-exon 50 junction or the 3′ exon 50 intron junction of the pre-mRNA of the DMD gene. In some instances, the PMO molecule hybridizes to a target region that is at either the 5′ intron-exon 49-51 junction or the 3′ exon 49-51 intron junction of the pre-mRNA of the DMD gene. In some instances, the PMO molecule hybridizes to a target region that is at either the 5′ intron-exon 49-52 junction or the 3′ exon 49-52 intron junction of the pre-mRNA of the DMD gene.

In some aspects, the PMO molecule comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs; 100-169.

In some aspects, the PMO molecule comprises a core sequence having at least 50%, 55%, 60%, 65%, 70%, 75%, 80%, 85%, 90%, 95%, 96%, 97%, 98%, 99%, or 100% sequence identity to a sequence selected from SEQ ID NOs; 100-169. In some aspects, the PMO molecule comprises a core sequence having at least 50%, 55%, 60%, 65%, 70%, 75%, 80%, 85%, 90%, 95%, 96%, 97%, 98%, 99%, or 100% sequence identity to a core sequence of one of SEQ ID NOs; 100-169. In some aspects the core sequence refers the nucleic acid sequence of positions 10-20 from the 5′ end, 11-21 from the 5′-end, 12-22 from the 5′ end, 9-21 from the 5′ end, 8-22 from the 5′ end. 7-23 from the 5′ end. In some aspects, the core sequence refers the nucleic acid sequence that is critical in hybridizing the target sequence.

In some aspects, the PMO molecule of the PMO-antibody conjugate comprises at least 10. 15, 16, 17, 18, 19, 20, 21, 22, 23, 24, 25, 26, 27, 28 or more contiguous nucleotides of a nucleic acid sequence selected from Table 1 or SEQ ID NOs; 100-143. In some cases, the PMO molecule further comprises 1, 2, 3, or 4 mismatches or no more than 1, 2, 3, or 4 mismatches from a nucleic acid sequence selected from Table 1 or SEQ ID NOs; 100-143.

In some aspects, the PMO molecule of the PMO-antibody conjugate comprises at least 10, 15, 16, 17, 18, 19, 20, 21, 22 or more contiguous nucleotides of a nucleic acid sequence selected from SEQ ID NOs; 144-147. In some cases, the PMO molecule further comprises 1, 2, 3, or 4 mismatches or no more than 1, 2, 3, or 4 mismatches from a nucleic acid sequence selected from SEQ ID NOs; 144-147.

In some aspects, the PMO molecule of the PMO-antibody conjugate comprises at least 10. 15, 16, 17, 18, 19, 20, 21, 22, 23, 24 or more contiguous nucleotides of a nucleic acid sequence selected from SEQ ID NOs; 148-160. In some cases, the PMO molecule further comprises 1, 2, 3, or 4 mismatches or no more than 1, 2, 3, or 4 mismatches from a nucleic acid sequence selected from SEQ ID NOs; 148-160.

In some aspects, the PMO molecule of the PMO-antibody conjugate comprises at least 10. 15, 16, 17, 18, 19. 20. 21. 22. 23. 24. 25. 26 or more contiguous nucleotides of a nucleic acid sequence selected from SEQ ID NOs; 161-169. In some cases, the PMO molecule further comprises 1, 2, 3, or 4 mismatches or no more than 1, 2, 3, or 4 mismatches from a nucleic acid sequence selected from SEQ ID NOs; 161-169.

In some aspects, the PMO molecule of the PMO-antibody conjugate comprises a sequence that binds to or hybridizes to exon 50. In some aspects, the PMO sequence of the PMO-antibody conjugate comprises a PMO sequence that binds to or hybridizes to SEQ ID NO; 200.

In some aspects, the PMO molecule of the PMO-antibody conjugate comprises a PMO sequence selected from the group consisting of SEQ ID NOs; 100-169. In some aspects, the PMO molecule of the PMO-antibody conjugate comprises a PMO sequence selected from the group consisting of Table 1 or SEQ ID NOs; 100-143. In some aspects, the PMO molecule of the PMO-antibody conjugate comprises a PMO sequence selected from the group consisting of SEQ ID NOs; 144-147. In some aspects, the PMO molecule of the PMO-antibody conjugate comprises a PMO sequence selected from the group consisting of SEQ ID NOs; 148-160. In some aspects, the PMO molecule of the PMO-antibody conjugate comprises a PMO sequence selected from the group consisting of SEQ ID NOs; 161-169.

In some aspects, the PMO molecule of the PMO-antibody conjugate comprises the PMO sequence of SEQ ID NO; 103. In some aspects, the PMO molecule of the PMO-antibody conjugate comprises the PMO sequence of SEQ ID NO; 113. In some aspects, the PMO molecule of the PMO-antibody conjugate comprises the PMO sequence of SEQ ID NO; 115. In some aspects, the PMO molecule of the PMO-antibody conjugate comprises the PMO sequence of SEQ ID NO; 116. In some aspects, the PMO molecule of the PMO-antibody conjugate comprises the PMO sequence of SEQ ID NO; 117. In some aspects, the PMO molecule of the PMO-antibody conjugate comprises the PMO sequence of SEQ ID NO; 118. In some aspects, the PMO molecule of the PMO-antibody conjugate comprises the PMO sequence of SEQ ID NO; 119. In some aspects, the PMO molecule of the PMO-antibody conjugate comprises the PMO sequence of SEQ ID NO; 120. In some aspects, the PMO molecule of the PMO-antibody conjugate comprises the PMO sequence of SEQ ID NO; 125. In some aspects, the PMO molecule of the PMO-antibody conjugate comprises the PMO sequence of SEQ ID NO; 145. In some aspects, the PMO molecule of the PMO-antibody conjugate comprises the PMO sequence of SEQ ID NO; 152. In some aspects, the PMO molecule of the PMO-antibody conjugate comprises the PMO sequence of SEQ ID NO; 153. In some aspects, the PMO molecule of the PMO-antibody conjugate comprises the PMO sequence of SEQ ID NO; 154. In some aspects, the PMO molecule of the PMO-antibody conjugate comprises the PMO sequence of SEQ ID NO; 155. In some aspects, the PMO molecule of the PMO-antibody conjugate comprises the PMO sequence of SEQ ID NO; 156. In some aspects, the PMO molecule of the PMO-antibody conjugate comprises the PMO sequence of SEQ ID NO; 157. In some aspects, the PMO molecule of the PMO-antibody conjugate comprises the PMO sequence of SEQ ID NO; 158. In some aspects, the PMO molecule of the PMO-antibody conjugate comprises the PMO sequence of SEQ ID NO; 159. In some aspects, the PMO molecule of the PMO-antibody conjugate comprises the PMO sequence of SEQ ID NO; 160. In some aspects, the PMO molecule of the PMO-antibody conjugate comprises the PMO sequence of SEQ ID NO; 161.

Tables 1, 2, and 3 list the PMO molecules of SEQ ID NOs; 100-169.

TABLE 1

| | | PMO | | SEQ |
|---|---|---|---|---|
| PMO | Target | Length | | ID |
| Name | Exon | (bp) | PMO Sequence (5′-3′) | NO: |
| Ex50-1 | 50 | 28 | GAGCTCAGATCTTCTAACTTCCTCTTCA | 100 |
| Ex50-2 | 50 | 28 | CAGAGCTCAGATCTTCTAACTTCCTCTT | 101 |
| Ex50-3 | 50 | 28 | CTCAGAGCTCAGATCTTCTAACTTCCTC | 102 |
| Ex50-4 | 50 | 28 | CACTCAGAGCTCAGATCTTCTAACTTCC | 103 |
| Ex50-5 | 50 | 28 | TCCACTCAGAGCTCAGATCTTCTAACTT | 104 |
| Ex50-6 | 50 | 28 | CTTCCACTCAGAGCTCAGATCTTCTAAC | 105 |
| Ex50-7 | 50 | 28 | GCCTTCCACTCAGAGCTCAGATCTTCTA | 106 |
| Ex50-8 | 50 | 28 | CCGCCTTCCACTCAGAGCTCAGATCTTC | 107 |
| Ex50-9 | 50 | 28 | TACCGCCTTCCACTCAGAGCTCAGATCT | 108 |
| Ex50-10 | 50 | 28 | TTTACCGCCTTCCACTCAGAGCTCAGAT | 109 |
| Ex50-11 | 50 | 28 | GGTTTACCGCCTTCCACTCAGAGCTCAG | 110 |
| Ex50-12 | 50 | 28 | ACGGTTTACCGCCTTCCACTCAGAGCTC | 111 |
| Ex50-13 | 50 | 28 | AAACGGTTTACCGCCTTCCACTCAGAGC | 112 |
| Ex50-14 | 50 | 28 | GTAAACGGTTTACCGCCTTCCACTCAGA | 113 |
| Ex50-15 | 50 | 28 | AAGTAAACGGTTTACCGCCTTCCACTCA | 114 |
| Ex50-16 | 50 | 28 | TGAAGTAAACGGTTTACCGCCTTCCACT | 115 |
| Ex50-17 | 50 | 28 | CTTGAAGTAAACGGTTTACCGCCTTCCA | 116 |
| Ex50-18 | 50 | 28 | CTCTTGAAGTAAACGGTTTACCGCCTTC | 117 |
| Ex50-19 | 50 | 28 | AGCTCTTGAAGTAAACGGTTTACCGCCT | 118 |
| Ex50-20 | 50 | 28 | TCAGCTCTTGAAGTAAACGGTTTACCGC | 119 |
| Ex50-21 | 50 | 28 | CCTCAGCTCTTGAAGTAAACGGTTTACC | 120 |
| Ex50-22 | 50 | 28 | GCCCTCAGCTCTTGAAGTAAACGGTTTA | 121 |
| Ex50-23 | 50 | 28 | TTGCCCTCAGCTCTTGAAGTAAACGGTT | 122 |
| Ex50-24 | 50 | 28 | CTTTGCCCTCAGCTCTTGAAGTAAACGG | 123 |
| Ex50-25 | 50 | 28 | TGCTTTGCCCTCAGCTCTTGAAGTAAAC | 124 |
| Ex50-26 | 50 | 28 | GCTGCTTTGCCCTCAGCTCTTGAAGTAA | 125 |
| Ex50-27 | 50 | 28 | AGGCTGCTTTGCCCTCAGCTCTTGAAGT | 126 |
| Ex50-28 | 50 | 28 | TCAGGCTGCTTTGCCCTCAGCTCTTGAA | 127 |
| Ex50-29 | 50 | 28 | GGTCAGGCTGCTTTGCCCTCAGCTCTTG | 128 |
| Ex50-30 | 50 | 28 | TAGGTCAGGCTGCTTTGCCCTCAGCTCT | 129 |
| Ex50-31 | 50 | 28 | GCTAGGTCAGGCTGCTTTGCCCTCAGCT | 130 |
| Ex50-32 | 50 | 28 | GAGCTAGGTCAGGCTGCTTTGCCCTCAG | 131 |
| Ex50-33 | 50 | 28 | AGGAGCTAGGTCAGGCTGCTTTGCCCTC | 132 |
| Ex50-34 | 50 | 28 | CCAGGAGCTAGGTCAGGCTGCTTTGCCC | 133 |
| Ex50-35 | 50 | 28 | GTCCAGGAGCTAGGTCAGGCTGCTTTGC | 134 |
| Ex50-36 | 50 | 28 | CAGTCCAGGAGCTAGGTCAGGCTGCTTT | 135 |
| Ex50-37 | 50 | 28 | GTCAGTCCAGGAGCTAGGTCAGGCTGCT | 136 |
| Ex50-38 | 50 | 28 | TGGTCAGTCCAGGAGCTAGGTCAGGCTG | 137 |
| Ex50-39 | 50 | 28 | AGTGGTCAGTCCAGGAGCTAGGTCAGGC | 138 |
| Ex50-40 | 50 | 28 | ATAGTGGTCAGTCCAGGAGCTAGGTCAG | 139 |
| Ex50-41 | 50 | 28 | CAATAGTGGTCAGTCCAGGAGCTAGGTC | 140 |
| Ex50-42 | 50 | 28 | TCCAATAGTGGTCAGTCCAGGAGCTAGG | 141 |
| Ex50-43 | 50 | 28 | GCTCCAATAGTGGTCAGTCCAGGAGCTA | 142 |
| Ex50-44 | 50 | 28 | AGGCTCCAATAGTGGTCAGTCCAGGAGC | 143 |

TABLE 2

| | | PMO | | SEQ |
|---|---|---|---|---|
| | Target | Length | | ID |
| PMO Name | Exon | (bp) | PMO Sequence (5′-3′) | NO: |
| hEx50_Ac9_28 mer | 50 | 28 | CCGCCTTCCACTCAGAGCTCAGATCTTC | 107 |
| hEx50_Ac11_28 mer | 50 | 28 | TACCGCCTTCCACTCAGAGCTCAGATCT | 108 |
| hEx50_Ac13_28 mer | 50 | 28 | TTTACCGCCTTCCACTCAGAGCTCAGAT | 109 |
| hEx50_Ac15_28 mer | 50 | 28 | GGTTTACCGCCTTCCACTCAGAGCTCAG | 110 |
| hEx50_Ac17_28 mer | 50 | 28 | ACGGTTTACCGCCTTCCACTCAGAGCTC | 111 |
| hEx50_Ac29_28 mer | 50 | 28 | CTCTTGAAGTAAACGGTTTACCGCCTTC | 117 |
| hEx50_Ac37_28 mer | 50 | 28 | GCCCTCAGCTCTTGAAGTAAACGGTTTA | 121 |
| hEx50_Ac69_28 mer | 50 | 28 | TGGTCAGTCCAGGAGCTAGGTCAGGCTG | 137 |
| hEx50_Ac71_28 mer | 50 | 28 | AGTGGTCAGTCCAGGAGCTAGGTCAGGC | 138 |
| hEx50_Ac73_28 mer | 50 | 28 | ATAGTGGTCAGTCCAGGAGCTAGGTCAG | 139 |
| hEx50_Ac79_28 mer | 50 | 28 | GCTCCAATAGTGGTCAGTCCAGGAGCTA | 142 |

TABLE 3

| | | PMO | | SEQ |
|---|---|---|---|---|
| | Target | Length | | ID |
| PMO Name | Exon | (bp) | PMO Sequence (5′-3′) | NO: |
| hEx50_Ac15_22 mer | 50 | 22 | CCGCCTTCCACTCAGAGCTCAG | 144 |
| hEx50_Ac17_22 mer | 50 | 22 | TACCGCCTTCCACTCAGAGCTC | 145 |
| hEx50_Ac73_22 mer | 50 | 22 | GTCAGTCCAGGAGCTAGGTCAG | 146 |
| hEx50_Ac75_22 mer | 50 | 22 | TGGTCAGTCCAGGAGCTAGGTC | 147 |
| hEx50_Ac9_24 mer | 50 | 24 | CTTCCACTCAGAGCTCAGATCTTC | 148 |
| hEx50_Ac11_24 mer | 50 | 24 | GCCTTCCACTCAGAGCTCAGATCT | 149 |
| hEx50_Ac13_24 mer | 50 | 24 | CCGCCTTCCACTCAGAGCTCAGAT | 150 |
| hEx50_Ac15_24 mer | 50 | 24 | TACCGCCTTCCACTCAGAGCTCAG | 151 |
| hEx50_Ac17_24 mer | 50 | 24 | TTTACCGCCTTCCACTCAGAGCTC | 152 |
| hEx50_Ac19_24 mer | 50 | 24 | GGTTTACCGCCTTCCACTCAGAGC | 153 |
| hEx50_Ac21_24 mer | 50 | 24 | ACGGTTTACCGCCTTCCACTCAGA | 154 |
| hEx50_Ac69_24 mer | 50 | 24 | CAGTCCAGGAGCTAGGTCAGGCTG | 155 |
| hEx50_Ac71_24 mer | 50 | 24 | GTCAGTCCAGGAGCTAGGTCAGGC | 156 |
| hEx50_Ac73_24 mer | 50 | 24 | TGGTCAGTCCAGGAGCTAGGTCAG | 157 |
| hEx50_Ac75_24 mer | 50 | 24 | AGTGGTCAGTCCAGGAGCTAGGTC | 158 |
| hEx50_Ac77_24 mer | 50 | 24 | ATAGTGGTCAGTCCAGGAGCTAGG | 159 |
| hEx50_Ac79_24 mer | 50 | 24 | CAATAGTGGTCAGTCCAGGAGCTA | 160 |
| hEx50_Ac9_26 mer | 50 | 26 | GCCTTCCACTCAGAGCTCAGATCTTC | 161 |
| hEx50_Ac11_26 mer | 50 | 26 | CCGCCTTCCACTCAGAGCTCAGATCT | 162 |
| hEx50_Ac13_26 mer | 50 | 26 | TACCGCCTTCCACTCAGAGCTCAGAT | 163 |
| hEx50_Ac15_26 mer | 50 | 26 | TTTACCGCCTTCCACTCAGAGCTCAG | 164 |
| hEx50_Ac17_26 mer | 50 | 26 | GGTTTACCGCCTTCCACTCAGAGCTC | 165 |
| hEx50_Ac69_26 mer | 50 | 26 | GTCAGTCCAGGAGCTAGGTCAGGCTG | 166 |
| hEx50_Ac71_26 mer | 50 | 26 | TGGTCAGTCCAGGAGCTAGGTCAGGC | 167 |
| hEx50_Ac73_26 mer | 50 | 26 | AGTGGTCAGTCCAGGAGCTAGGTCAG | 168 |
| hEx50_Ac75_26 mer | 50 | 26 | ATAGTGGTCAGTCCAGGAGCTAGGTC | 169 |
| hEx50_Ac9_28 mer | 50 | 28 | CCGCCTTCCACTCAGAGCTCAGATCTTC | 107 |
| hEx50_Ac13_28 mer | 50 | 28 | TTTACCGCCTTCCACTCAGAGCTCAGAT | 109 |
| hEx50_Ac73_28 mer | 50 | 28 | ATAGTGGTCAGTCCAGGAGCTAGGTCAG | 139 |

In some aspects, the polynucleic acid molecule is an antisense oligonucleotide (ASO) or phosphorodiamidate morpholino oligonucleotide (PMO) molecule.

In some aspects, the ASO or PMO molecule is from about 10 to about 50 nucleotides in length. In some instances, the ASO or PMO molecule is from about 10 to about 30, from about 15 to about 30, from about 18 to about 30, from about 20 to about 30, from about 22 to about 30, from about 24 to about 30, from about 26 to about 30, from about 28 to about 30, from about 24 to about 28, from about 26 to about 28, or from about 24 to about 26.

In some aspects, the polynucleic acid molecule comprises natural, synthetic, or artificial nucleotide analogues or bases. In some cases, the ASO molecule or the PMO molecule of the polynucleic acid molecule-antibody conjugate (e.g., PMO-antibody conjugate or ASO-antibody conjugate) comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some aspects, the nucleotide analogues or artificial nucleotide bases comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moieties include, but are not limited to, halogens, sulfurs, thiols, thioethers, thioesters, amines (primary, secondary, or tertiary), amides, ethers, esters, alcohols, and oxygen. In some instances, the alkyl moiety further comprises a modification. In some instances, the modification comprises an azo group, a keto group, an aldehyde group, a carboxyl group, a nitro group, a nitroso group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety. Exemplary chemical structures of a 2′-O-methyl modification of an adenosine molecule and 2′O-methoxyethyl modification of a uridine are illustrated below.

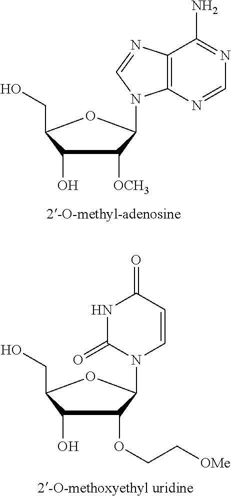

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

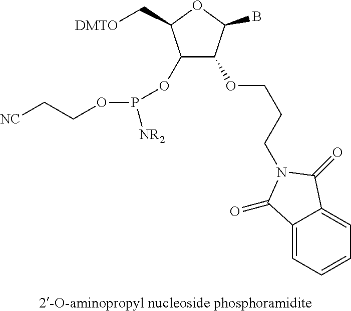

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

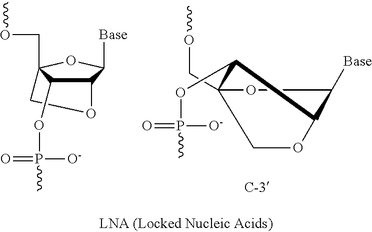

In some instances, the modification at the 2′ hydroxyl group comprises ethylene nucleic acids (ENA) such as for example 2′-4′-ethylene-bridged nucleic acid, which locks the sugar conformation into a C3′-endo sugar puckering conformation. ENAs are part of the bridged nucleic acids class of modified nucleic acids that also comprises LNA. Exemplary chemical structures of the ENA and bridged nucleic acids are illustrated below.

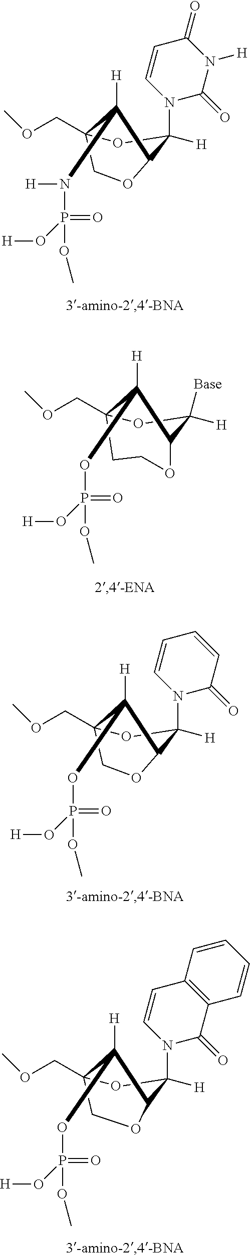

In some aspects, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some aspects, nucleotide analogues comprise modified bases such as, but not limited to, 5-propynyluridine, 5-propynylcytidine, 6-methyladenine, 6-methylguanine, N, N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholinos or phosphorodiamidate morpholino oligomers (PMOs) comprise synthetic molecules whose structures mimic natural nucleic acid structures by deviating from the normal sugar and phosphate structures. In some instances, the five-member ribose ring is substituted with a six-member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

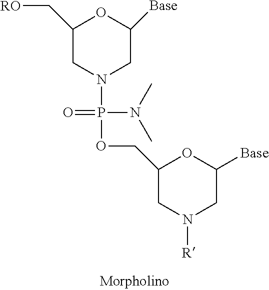

In some aspects, the peptide nucleic acid (PNA) does not contain a sugar ring or phosphate linkage and the bases are attached and appropriately spaced by oligoglycine-like molecules, therefore, eliminating a backbone charge.

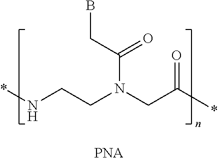

In some aspects, one or more modifications optionally occur at the internucleotide linkage. In some instances, modified internucleotide linkages include, but are not limited to, phosphorothioates, phosphorodithioates, methylphosphonates, 5′-alkylenephosphonates, 5′-methylphosphonates, 3′-alkylene phosphonates, borontrifluoridates, borano phosphate esters and selenophosphates with 3′-5′ linkages or 2′-5′ linkages, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. An exemplary PS ASO is illustrated below.

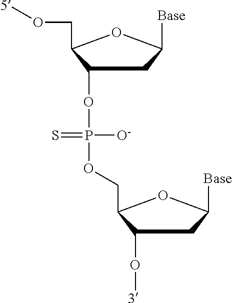

In some instances, the modification is a methyl or thiol modification such as methylphosphonate or thiolphosphonate modification. An exemplary thiolphosphonate nucleotide (left) and an methylphosphonate nucleotide (right) are illustrated below.

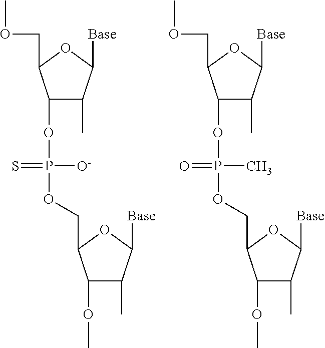

In some instances, a modified nucleotide includes, but is not limited to, 2′-fluoro N3-P5′-phosphoramidites illustrated as:

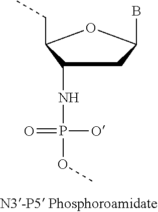

In some instances, a modified nucleotide includes, but is not limited to, hexitol nucleic acid (or l′, 5′-anhydrohexitol nucleic acids (HNA)) illustrated as:

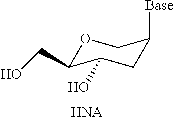

In some aspects, a nucleotide analogue or artificial nucleotide base described above comprises a 5′-vinylphosphonate modified nucleotide with a modification at a 5′ hydroxyl group of the ribose moiety. In some aspects, the 5′-vinylphosphonate modified nucleotide is selected from the nucleotides provided below, wherein X is O or S; and B is a heterocyclic base moiety.

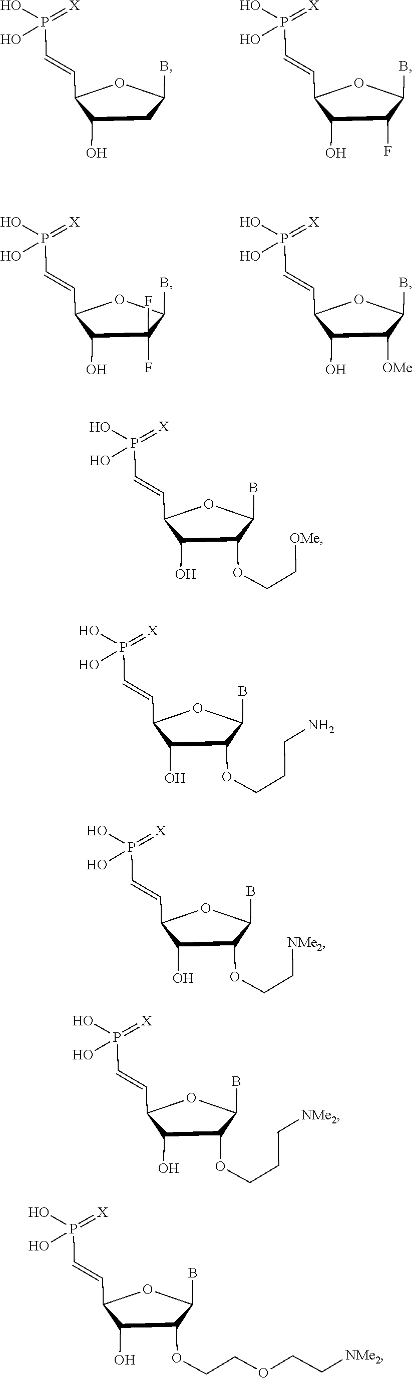

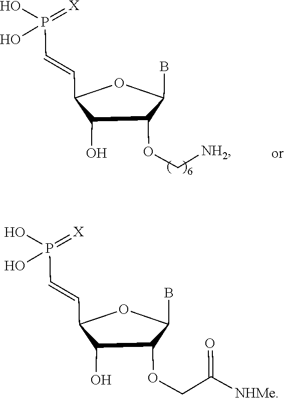

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some instances, the 5′-vinylphosphonate modified nucleotide is further modified at the 2′ hydroxyl group in a locked or bridged ribose modification (e.g., locked nucleic acid or LNA) in which the oxygen molecule bound at the 2′ carbon is linked to the 4′ carbon by a methylene group, thus forming a 2′-C, 4′-C-oxy-methylene-linked bicyclic ribonucleotide monomer. Exemplary representations of the chemical structure of 5′-vinylphosphonate modified LNA are illustrated below, wherein X is O or S; B is a heterocyclic base moiety; and J is an internucleotide linking group linking to the adjacent nucleotide of the polynucleotide.

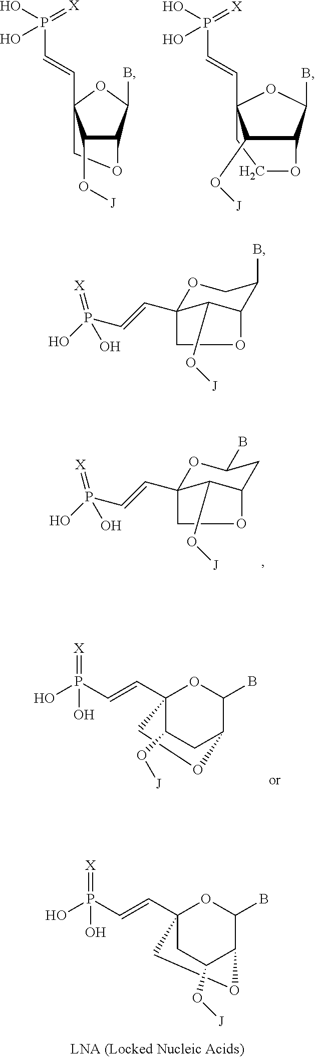

In some aspects, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some aspects, a nucleotide analogue comprises a modified base such as, but not limited to, 5-propynyluridine, 5-propynylcytidine, 6-methyladenine, 6-methylguanine, N, N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. 5′-Vinylphosphonate modified nucleotides may also include those nucleotides that are modified with respect to the sugar moiety, as well as 5′-vinylphosphonate modified nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, a 5′-vinylphosphonate modified nucleotide analogue further comprises a morpholino, a peptide nucleic acid (PNA), a methylphosphonate nucleotide, a thiolphosphonate nucleotide, a 2′-fluoro N3-P5′-phosphoramidite, or a l′, 5′-anhydrohexitol nucleic acid (HNA). Morpholinos or phosphorodiamidate morpholino oligomers (PMOs) comprise synthetic molecules whose structures mimic natural nucleic acid structure but deviate from the normal sugar and phosphate structures. In some instances, the five member ribose ring is substituted with a six-member morpholino ring containing four carbons, one nitrogen, and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides. A non-limiting example of a 5′-vinylphosphonate modified morpholino oligonucleotide is illustrated below, wherein B is a heterocyclic base moiety.

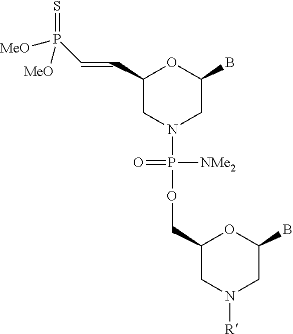

In some aspects, a 5′-vinylphosphonate modified morpholino or PMO described above is a PMO comprising a positive or cationic charge. In some instances, the PMO is PMOplus (Sarepta). PMOplus refers to phosphorodiamidate morpholino oligomers comprising any number of (1-piperazino)phosphinylideneoxy, (1-(4-(omega-guanidino-alkanoyl))-piperazino) phosphinylideneoxy linkages (e.g., as such those described in PCT Publication No. WO2008/036127. In some cases, the PMO is a PMO described in U.S. Pat. No. 7,943,762.

In some aspects, a morpholino or PMO described above is a PMO-X (Sarepta). In some cases, PMO-X refers to phosphorodiamidate morpholino oligomers comprising at least one linkage or at least one of the disclosed terminal modifications, such as those disclosed in PCT Publication No. WO2011/150408 and U.S. Publication No. 2012/0065169.

In some aspects, a morpholino or PMO described above is a PMO as described in Table 5 of U.S. Publication No. 2014/0296321.

Exemplary representations of the chemical structure of 5′-vinylphosphonate modified nucleic acids are illustrated below, wherein X is O or S: B is a heterocyclic base moiety; and J is an internucleotide linkage.

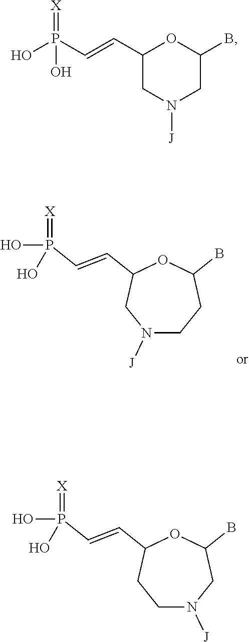

In some aspects, one or more modifications of the 5′-vinylphosphonate modified oligonucleotide optionally occur at the internucleotide linkage. In some instances, modified internucleotide linkages include, but is not limited to, phosphorothioates; phosphorodithioates: methylphosphonates; 5′-alkylenephosphonates; 5′-methylphosphonate; 3′-alkylene phosphonates: borontrifluoridates; borano phosphate esters and selenophosphates with 3′-5′linkages or 2′-5′linkages; phosphotriesters; thionoalkylphosphotriesters: hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides: carbonates; carbamates; methylenehydrazos: methylenedimethylhydrazos; formacetals; thioformacetals: oximes; methyleneiminos: methylenemethyliminos; thioamidates; linkages with riboacetyl groups; aminoethyl glycine: silyl or siloxane linkages; alkyl or cycloalkyl linkages with or without heteroatoms of, for example, 1 to 10 carbons that are saturated or unsaturated and/or substituted and/or contain heteroatoms: linkages with morpholino structures, amides, or polyamides wherein the bases are attached to the aza nitrogens of the backbone directly or indirectly; and combinations thereof.

In some instances, the modification is a methyl or thiol modification such as methylphosphonate or thiolphosphonate modifications. An exemplary thiolphosphonate nucleotide (left), phosphorodithioates (center) and methylphosphonate nucleotide (right) are illustrated below.

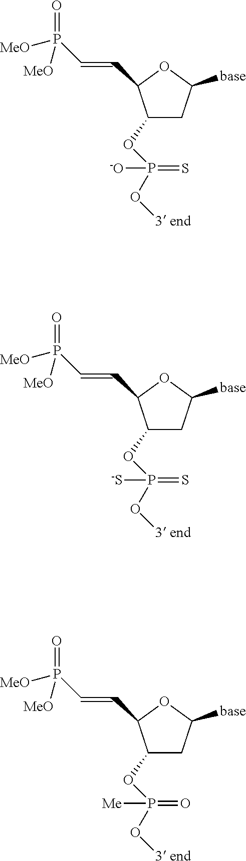

In some instances, a 5′-vinylphosphonate modified nucleotide includes, but is not limited to, phosphoramidites illustrated as:

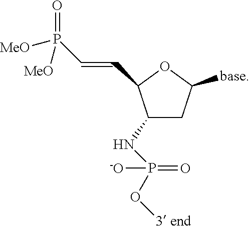

In some instances, the modified internucleotide linkage is a phosphorodiamidate linkage. A non-limiting example of a phosphorodiamidate linkage with a morpholino system is shown below.

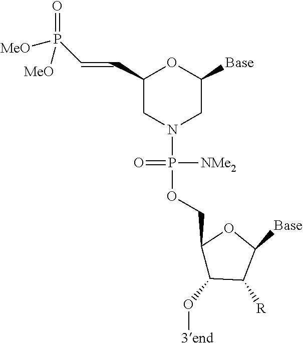

In some instances, the modified internucleotide linkage is a methylphosphonate linkage. A non-limiting example of a methylphosphonate linkage is shown below.

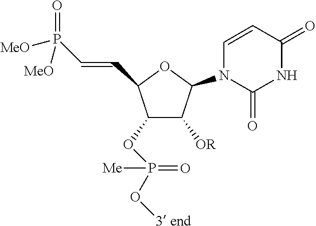

In some instances, the modified internucleotide linkage is an amide linkage. A non-limiting example of an amide linkage is shown below.

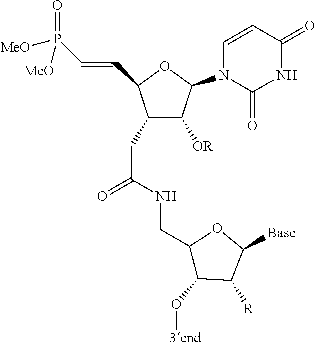

In some instances, a 5′-vinylphosphonate modified nucleotide includes, but is not limited to, the modified nucleic acid illustrated below.

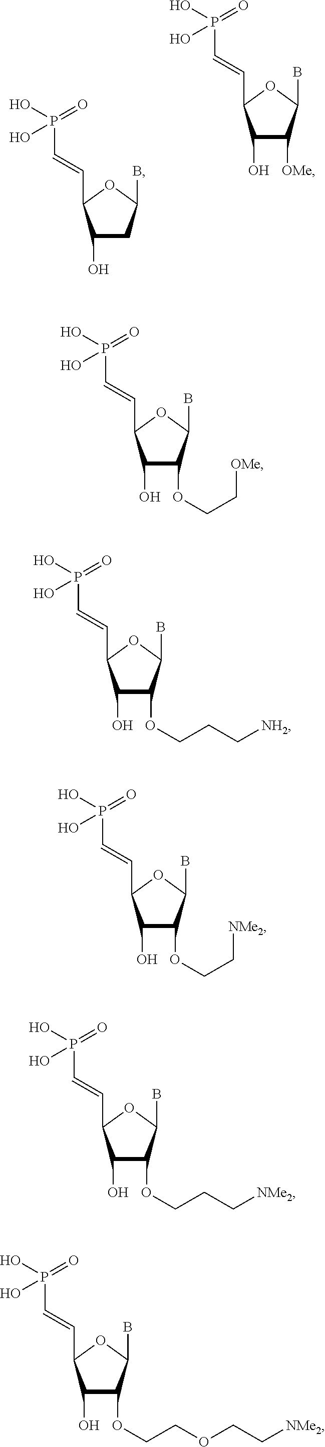

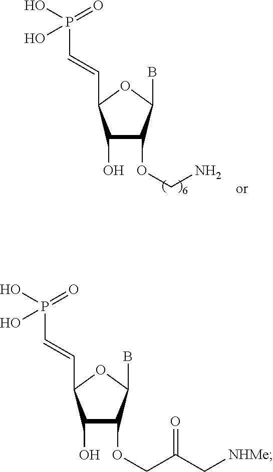

wherein B is a heterocyclic base moiety.

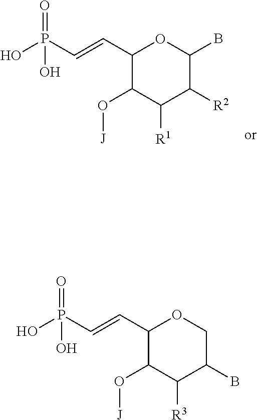

wherein B is a heterocyclic base moiety;
R4, and R5 are independently selected from hydrogen, halogen, alkyl or alkoxy; and
J is an internucleotide linking group linking to the adjacent nucleotide of the polynucleotide.

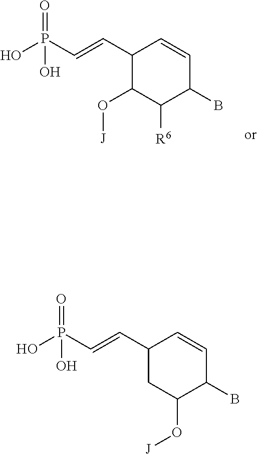

wherein B is a heterocyclic base moiety;
R6 is selected from hydrogen, halogen, alkyl or alkoxy; and
J is an internucleotide linking group linking to the adjacent nucleotide of the polynucleotide.

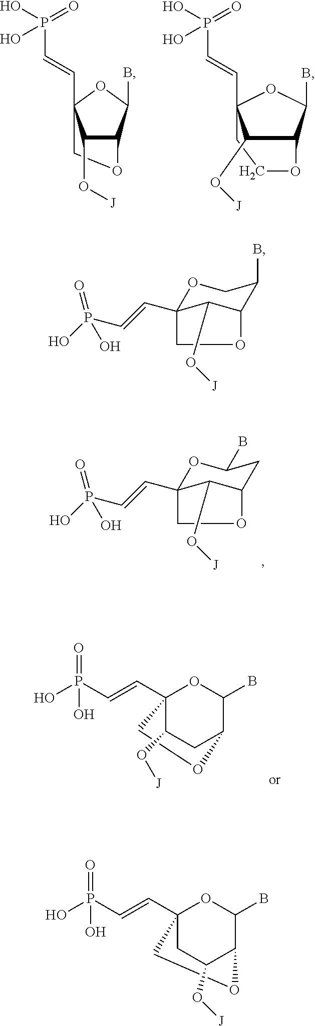

wherein B is a heterocyclic base moiety; and
J is an internucleotide linking group linking to the adjacent nucleotide of the polynucleotide.

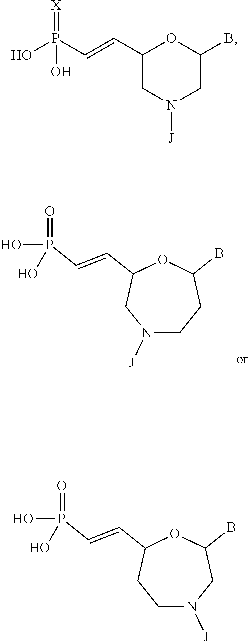

wherein B is a heterocyclic base moiety; and
J is an internucleotide linking group linking to the adjacent nucleotide of the polynucleotide.

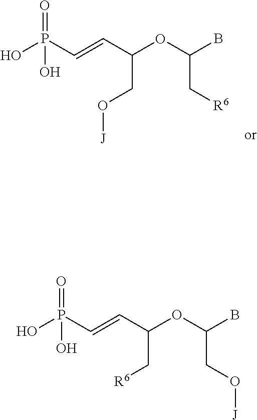

wherein B is a heterocyclic base moiety;
R6 is selected from hydrogen, halogen, alkyl or alkoxy; and
J is an internucleotide linking group linking to the adjacent nucleotide of the polynucleotide.

In some aspects, the PMO molecule of the PMO-antibody conjugate comprises a plurality of phosphorodiamidate morpholino oligomers or a plurality of peptide nucleic acid-modified non-natural nucleotides, and optionally comprises at least one inverted abasic moiety. In some instances, the PMO molecule comprises at least 6, 7, 8, 9, 10, 11, 12, 13, 14, 15, 16, 17, 18, 19, 20, or more phosphorodiamidate morpholino oligomer-modified non-natural nucleotides. In some instances, the PMO molecule comprises 100% phosphorodiamidate morpholino oligomer-modified non-natural nucleotides.

In some instances, the PMO molecule of the PMO-antibody conjugate comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification.

In some cases, one or more of the artificial nucleotide analogues described herein are resistant toward nucleases such as for example ribonuclease such as RNase H, deoxyribonuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy. 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, 2′-O-methyl modified polynucleic acid molecule is nuclease resistant (e.g., RNase H, DNase, 5′-3′ exonuclease or 3′-5′ exonuclease resistance). In some instances, 2′O-methoxyethyl (2′-O-MOE) modified polynucleic acid molecules are nuclease resistant (e.g., RNase H, DNase, 5′-3′ exonuclease or 3′-5′ exonuclease resistance). In some instances, 2′-O-aminopropyl modified polynucleic acid molecules are nuclease resistant (e.g., RNase H. DNase, 5′-3′ exonuclease or 3′-5′ exonuclease resistance). In some instances, 2′-deoxy modified polynucleic acid molecules are nuclease resistant (e.g., RNase H, DNase, 5′-3′ exonuclease or 3′-5′ exonuclease resistance). In some instances, 2′-deoxy-2′-fluoro modified polynucleic acid molecules are nuclease resistant (e.g., RNase H, DNase, 5′-3′ exonuclease or 3′-5′ exonuclease resistance). In some instances, 2′-O-aminopropyl (2′-O-AP) modified polynucleic acid molecules are nuclease resistant (e.g., RNase H, DNase, 5′-3′ exonuclease or 3′-5′ exonuclease resistance). In some instances, 2′-O-dimethylaminoethyl (2′-O-DMAOE) modified polynucleic acid molecules are nuclease resistant (e.g., RNase H. DNase, 5′-3′ exonuclease or 3′-5′ exonuclease resistance). In some instances, 2′-O-dimethylaminopropyl (2′-O-DMAP) modified polynucleic acid molecules are nuclease resistant (e.g., RNase H, DNase, 5′-3′ exonuclease or 3′-5′ exonuclease resistance). In some instances, 2′-O-dimethylaminoethyloxyethyl (2′-O-DMAEOE) modified polynucleic acid molecules are nuclease resistant (e.g., RNase H, DNase, 5′-3′ exonuclease or 3′-5′ exonuclease resistance). In some instances, 2′-O—N-methylacetamido (2′-O-NMA) modified polynucleic acid molecules are nuclease resistant (e.g., RNase H, DNase, 5′-3′ exonuclease or 3′-5′ exonuclease resistance). In some instances, LNA modified polynucleic acid molecules are nuclease resistant (e.g., RNase H, DNase, 5′-3′ exonuclease or 3′-5′ exonuclease resistance). In some instances, ENA modified polynucleic acid molecules are nuclease resistant (e.g., RNase H, DNase, 5′-3′ exonuclease or 3′-5′ exonuclease resistance). In some instances, HNA modified polynucleic acid molecules are nuclease resistant (e.g., RNase H, DNase, 5′-3′ exonuclease or 3′-5′ exonuclease resistance). In some instances, morpholinos are nuclease resistant (e.g., RNase H, DNase, 5′-3′ exonuclease or 3′-5′ exonuclease resistance). In some instances, PNA modified polynucleic acid molecules are resistant to nucleases (e.g., RNase H, DNase, 5′-3′ exonuclease or 3′-5′ exonuclease resistance). In some instances, methylphosphonate nucleotides modified polynucleic acid molecule are nuclease resistant (e.g., RNase H, DNase, 5′-3′ exonuclease or 3′-5′ exonuclease resistance). In some instances, thiolphosphonate nucleotide modified polynucleic acid molecules are nuclease resistant (e.g., RNase H, DNase, 5′-3′ exonuclease or 3′-5′ exonuclease resistance). In some instances, polynucleic acid molecules comprising 2-fluoro N3-P5′-phosphoramidites are nuclease resistant (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

### Polynucleic Acid Molecule Synthesis

In some aspects, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex formed between the polynucleic acid molecule and target nucleic acids. Exemplary methods include those described in: U.S. Pat. Nos. 5,142,047:5.185.444:5.889.136; 6,008.400; and 6.111.086; PCT Publication No. WO2009099942: or European Publication No. 1579015. Additional exemplary methods include those described in: Griffey et al . . . “2”-O-aminopropyl ribonucleotides: a zwitterionic modification that enhances the exonuclease resistance and biological activity of antisense oligonucleotides.” J. Med. Chem. 39 (26); 5100-5109 (1997)): Obika, et al. “Synthesis of 2′-0,4′-C-methyleneuridine and-cytidine. Novel bicyclic nucleosides having a fixed C3.-endo sugar puckering”. Tetrahedron Letters 38 (50); 8735 (1997): Koizumi. M. “ENA oligonucleotides as therapeutics”. Current opinion in molecular therapeutics 8 (2); 144-149 (2006); and Abramova et al . . . “Novel oligonucleotide analogues based on morpholino nucleoside subunits-antisense technologies: new chemical possibilities.” Indian Journal of Chemistry 48B; 1721-1726 (2009). Alternatively, the polynucleic acid molecule is produced biologically using an expression vector into which a polynucleic acid molecule has been subcloned in an antisense orientation (i.e., RNA transcribed from the inserted polynucleic acid molecule will be of an antisense orientation to a target polynucleic acid molecule of interest).

In some aspects, a polynucleic acid molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

Additional modification methods for incorporating, for example, sugar, base and phosphate modifications include: Eckstein et al., International Publication PCT No. WO 92/07065: Perrault et al. Nature. 1990, 344. 565-568; Pieken et al. Science. 1991. 253. 314-317: Usman and Cedergren. Trends in Biochem. Sci., 1992. 17. 334-339; Usman et al. International Publication PCT No. WO 93/15187: Sproat. U.S. Pat. No. 5,334,711 and Beigelman et al., 1995. J. Biol. Chem . . . 270. 25702: Beigelman et al., International PCT publication No. WO 97/26270; Beigelman et al., U.S. Pat. No. 5,716,824; Usman et al., U.S. Pat. No. 5,627,053: Woolf et al., International PCT Publication No. WO 98/13526: Thompson et al., U.S. Ser. No. 60/082,404 which was filed on Apr. 20. 1998: Karpeisky et al., 1998. Tetrahedron Lett . . . 39. 1131: Earnshaw and Gait. 1998. Biopolymers (Nucleic Acid Sciences), 48. 39-55: Verma and Eckstein. 1998. Annu. Rev. Biochem . . . 67. 99-134; and Burlina et al., 1997. Bioorg. Med. Chem . . . 5. 1999-2010. Such publications describe general methods and strategies to determine the location of incorporation of sugar, base and/or phosphate modifications and the like into nucleic acid molecules without modulating catalysis.

In some instances, while chemical modification of the polynucleic acid molecule internucleotide linkages with phosphorothioate, phosphorodithioate, and/or 5′-methylphosphonate linkages improves stability, excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

### Antibody

In some aspects, the antibody or antigen binding fragment thereof comprises a humanized antibody or antigen binding fragment thereof, murine antibody or antigen binding fragment thereof, chimeric antibody or antigen binding fragment thereof, monoclonal antibody or antigen binding fragment thereof, monovalent Fab′, divalent Fab2, F (ab)′3 fragments, single-chain variable fragment (scFv), bis-scFv, (scFv) 2, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or antigen binding fragment thereof, or a chemically modified derivative thereof.

In some instances, the antibody is an anti-transferrin receptor (anti-CD71) antibody or antigen binding fragment thereof. In some cases, the anti-transferrin receptor antibody is a humanized antibody or antigen binding fragment thereof. In other cases, the anti-transferrin receptor antibody is a chimeric antibody or antigen binding fragment thereof. In additional cases, the anti-transferrin receptor antibody is a monovalent, a divalent, or a multi-valent antibody or antigen binding fragment thereof. In some aspects, exemplary anti-transferrin receptor antibodies or antigen binding fragments thereof include MAB5746 from R&D Systems, AHP858 from Bio-Rad Laboratories, A80-128A from Bethyl Laboratories, Inc., and T2027 from MilliporeSigma. In some aspects, the anti-transferrin receptor antibody or antigen binding fragment thereof includes the antibodies disclosed in U.S. Pat. No. 10,913,800 or U.S. Pat. No. 11,028,179.

In some aspects, suitable anti-transferrin receptor antibodies as described herein are disclosed in, e.g., U.S. Pat. Nos. 10,913,800, 10,881,743, 11,446,387, 11,555,190, 11,912,779, 10,994,020, 12,071,621, U.S. patent application Ser. No. 18/755,579. U.S, patent application Ser. No. 18/759,724, or U.S, patent application Ser. No. 18/903,935. The content of these US patents and US patent applications are incorporated herein by reference in their entireties.

Other suitable anti-transferrin receptor antibodies are disclosed in US Patent Publication No. 2023/0285586, U.S. Pat. No. 11,771,776, US Patent Publication No. 2023/0144436, US Patent Publication No. 2024/0016952, international patent publication No. WO2023/201332, US Patent Publication No. 2023/0256112, US Patent Publication No. 2023/0113823, US Patent Publication No. 2023/010379, international patent publication No. WO2024/006976, and international patent publication No. WO2024/036096, each of which is incorporated herein by reference in its entirety.

In some instances, the anti-transferrin receptor antibody comprises a variable heavy chain (VH) region and a variable light chain (VL) region, wherein the VH region comprises an HCDR1 sequence comprising or consisting of a sequence of SEQ ID NO; 17: an HCDR2 sequence comprising or consisting of a sequence of EINPIXIGRSNYAX2KFQG (SEQ ID NO: 12), wherein X1 is selected from N or Q and X2 is selected from Q or E; and an HCDR3 sequence comprising or consisting of a sequence of SEQ ID NO; 19.

In some aspects, the VH region of the anti-transferring antibody comprises HCDR1, HCDR2, and HCDR3 sequences selected from Table 4.

TABLE 4

| | | SEQ ID | | SEQ ID | | SEQ ID |
|---|---|---|---|---|---|---|
| Name | HCDR1 | NO: | HCDR2 | NO: | HCDR3 | NO: |
| 13E4_VH1 | YTFTNYWMH | 17 | EINPINGRSNYAQKFQG | 18 | GTRAMHY | 19 |
| 13E4_VH2* | YTFTNYWMH | 17 | EINPINGRSNYAEKFQG | 20 | GTRAMHY | 19 |
| 13E4_VH3 | YTFTNYWMH | 17 | EINPIQGRSNYAEKFQG | 21 | GTRAMHY | 19 |

*13E4_VH2 shares the same HCDR1, HCDR2, and HCDR3 sequences with anti-transferrin receptor antibody 13E4 VH4

In some aspects, the VH region comprises an HCDR1 sequence comprising or

consisting of a sequence of SEQ ID NO; 17: an HCDR2 sequence comprising or consisting of a sequence of SEQ ID NO; 18, 20, or 21; and an HCDR3 sequence comprising or consisting of a sequence of SEQ ID NO; 19. In some instances, the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19. In some instances, the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19. In some instances, the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19. In some instances, the VH region comprises an HCDR1 sequence comprising or consisting of NYWMH (SEQ ID NO; 67), an HCDR2 sequence comprising or consisting of a sequence of SEQ ID NO; 20, and an HCDR3 sequence comprising or consisting of a sequence of SEQ ID NO; 19.

In some aspects, the VL region of the anti-transferrin receptor antibody comprises an LCDR1 sequence comprising or consisting of a sequence of RTSENIYX3NLA (SEQ ID NO: 13), an LCDR2 sequence comprising or consisting of a sequence of AX4TNLAX5 (SEQ ID NO; 14), and an LCDR3 sequence comprising or consisting of a sequence of QHFWGTPLTX6 (SEQ ID NO; 15), wherein X3 is selected from N or S, X4 is selected from A or G, X5 is selected from D or E, and X6 is present or absent, and if present, is F.

In some aspects, the VL region of the anti-transferrin receptor antibody comprises LCDR1, LCDR2, and LCDR3 sequences selected from Table 5.

TABLE 5

| | | SEQ ID | | SEQ ID | | SEQ ID |
|---|---|---|---|---|---|---|
| Name | LCDR1 | NO: | LCDR2 | NO: | LCDR3 | NO: |
| 13E4_VL1* | RTSENIYNNLA | 22 | AATNLAD | 23 | QHFWGTPLT | 24 |
| 13E4_VL3 | RTSENIYNNLA | 22 | AATNLAE | 25 | QHFWGTPLTF | 26 |
| 13E4_VL4 | RTSENIYSNLA | 27 | AGTNLAD | 28 | QHFWGTPLTF | 26 |

*13E4_VL1 shares the same LCDR1, LCDR2, and LCDR3 sequences with anti-transferrin receptor antibody 13E4_VL2

In some instances, the VL region comprises an LCDR1 sequence comprising or consisting of a sequence of SEQ ID NO; 13, an LCDR2 sequence comprising or consisting of a sequence of SEQ ID NO; 23, 25, or 28, and an LCDR3 sequence comprising or consisting of a sequence of SEQ ID NO; 24 or 26, wherein X3 is selected from N or S.

In some instances, the VL region comprises an LCDR1 sequence comprising or consisting of a sequence of SEQ ID NO; 22 or 27, an LCDR2 sequence comprising or consisting of a sequence of SEQ ID NO; 14, and an LCDR3 sequence comprising or consisting of a sequence of SEQ ID NO; 24 or 26, wherein X4 is selected from A or G, and X5 is selected from D or E.

In some instances, the VL region comprises an LCDR1 sequence comprising or consisting of a sequence of SEQ ID NO; 22 or 27, an LCDR2 sequence comprising or consisting of a sequence of SEQ ID NO; 23, 25, or 28, and an LCDR3 sequence comprising or consisting of a sequence of SEQ ID NO; 15, wherein X6 is present or absent, and if present, is F.

In some instances, the VL region comprises an LCDR1 sequence comprising or consisting of a sequence of SEQ ID NO; 22, an LCDR2 sequence comprising or consisting of a sequence of AATNLAX5 (SEQ ID NO; 16), and an LCDR3 sequence comprising or consisting of a sequence of SEQ ID NO; 15, wherein X5 is selected from D or E and X6 is present or absent, and if present, is F.

In some instances, the VL region comprises an LCDR1 sequence comprising or consisting of a sequence of SEQ ID NO; 22, an LCDR2 sequence comprising or consisting of a sequence of SEQ ID NO; 23, and an LCDR3 sequence comprising or consisting of a sequence of SEQ ID NO; 24.

In some instances, the VL region comprises an LCDR1 sequence comprising or consisting of a sequence of SEQ ID NO; 22, an LCDR2 sequence comprising or consisting of a sequence of SEQ ID NO; 25, and an LCDR3 sequence comprising or consisting of a sequence of SEQ ID NO; 26.

In some instances, the VL region comprises an LCDR1 sequence comprising or consisting of a sequence of SEQ ID NO; 27, an LCDR2 sequence comprising or consisting of a sequence of SEQ ID NO; 28, and an LCDR3 sequence comprising or consisting of a sequence of SEQ ID NO; 26.

In some aspects, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein X1 is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 13, an LCDR2 sequence comprising or consisting of a sequence of SEQ ID NO; 14, and an LCDR3 sequence comprising or consisting of a sequence of SEQ ID NO; 15, wherein X3 is selected from N or S. X4 is selected from A or G, X5 is selected from D or E, and X6 is present or absent, and if present, is F.

In some instances, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein X1 is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 13, an LCDR2 sequence comprising or consisting of a sequence of SEQ ID NO; 23, 25, or 28, and an LCDR3 sequence comprising or consisting of a sequence of SEQ ID NO; 24 or 26, wherein X3 is selected from N or S.

In some instances, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein X1 is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 14, and an LCDR3 sequence comprising or consisting of a sequence of SEQ ID NO; 24 or 26, wherein X4 is selected from A or G, and X5 is selected from D or E.

In some instances, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein X1 is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 23. 25, or 28, and an LCDR3 sequence comprising or consisting of a sequence of SEQ ID NO; 15, wherein X6 is present or absent, and if present, is F.

In some instances, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein X1 is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 16, and an LCDR3 sequence comprising or consisting of a sequence of SEQ ID NO; 15, wherein X5 is selected from D or E and X6 is present or absent, and if present, is F.

In some instances, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein X1 is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 23, and an LCDR3 sequence comprising or consisting of a sequence of SEQ ID NO; 24.

In some instances, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein XI is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 25, and an LCDR3 sequence comprising or consisting of a sequence of SEQ ID NO; 26.

In some instances, the anti-transferrin receptor antibody comprises a VH region and a VL region, wherein the VH region comprises an HCDR1 sequence comprising or consisting of a sequence of SEQ ID NO; 17: an HCDR2 sequence comprising or consisting of a sequence of SEQ ID NO; 12, wherein X1 is selected from N or Q and X2 is selected from Q or E; and an HCDR3 sequence comprising or consisting of a sequence of SEQ ID NO; 19; and the VL region comprises an LCDR1 sequence comprising or consisting of a sequence of SEQ ID NO; 27, an LCDR2 sequence comprising or consisting of a sequence of SEQ ID NO; 28, and an LCDR3 sequence comprising or consisting of a sequence of SEQ ID NO; 26.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19; and the VL region comprises an LCDR1 sequence comprising or consisting of a sequence of SEQ ID NO; 13, an LCDR2 sequence comprising or consisting of a sequence of SEQ ID NO; 23. 25, or 28, and an LCDR3 sequence comprising or consisting of a sequence of SEQ ID NO; 24 or 26, wherein X3 is selected from N or S.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 14, and an LCDR3 sequence comprising or consisting of a sequence of SEQ ID NO; 24 or 26, wherein X4 is selected from A or G, and X5 is selected from D or E.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 23. 25, or 28, and LCDR3 sequence comprising or consisting of a sequence of SEQ ID NO; 15, wherein X6 is present or absent, and if present, is F.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 16, and an LCDR3 sequence comprising or consisting of a sequence of SEQ ID NO; 15, wherein X5 is selected from D or E and X6 is present or absent, and if present, is F.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 23, and an LCDR3 sequence comprising or consisting of a sequence of SEQ ID NO; 24.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 21, and an LCDR3 sequence comprising or consisting of a sequence of SEQ ID NO; 26.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 18, and an HCDR3 sequence comprising or consisting of a sequence of SEQ ID NO; 19; and the VL region comprises an LCDR1 sequence comprising or consisting of a sequence of SEQ ID NO; 27, an LCDR2 sequence comprising or consisting of a sequence of SEQ ID NO; 28, and an LCDR3 sequence comprising or consisting of a sequence of SEQ ID NO; 26.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 13, an LCDR2 sequence comprising or consisting of a sequence of SEQ ID NO; 23. 25, or 28, and an LCDR3 sequence comprising or consisting of a sequence of SEQ ID NO; 24 or 26, wherein X3 is selected from N or S.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 14, and an LCDR3 sequence comprising or consisting of a sequence of SEQ ID NO; 24 or 26, wherein X4 is selected from A or G, and X5 is selected from D or E.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 23. 25 or 28, and an LCDR3 sequence comprising or consisting of a sequence of SEQ ID NO; 15, wherein X6 is present or absent, and if present, is F.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 16, and an LCDR3 sequence comprising or consisting of a sequence of SEQ ID NO; 15, wherein X5 is selected from D or E and X6 is present or absent, and if present, is F.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 23, and an LCDR3 sequence comprising or consisting of a sequence of SEQ ID NO; 24.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 25, and an LCDR3 sequence comprising or consisting of a sequence of SEQ ID NO; 26.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 27, an LCDR2 sequence comprising or consisting of a sequence of SEQ ID NO; 28, and an LCDR3 sequence comprising or consisting of a sequence of SEQ ID NO; 26.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19; and the VL region comprises an LCDR1 sequence comprising or consisting of a sequence of SEQ ID NO; 13, an LCDR2 sequence comprising or consisting of a sequence of SEQ ID NO; 23. 25, or 28, and an LCDR3 sequence comprising or consisting of a sequence of SEQ ID NO; 24 or 26, wherein X3 is selected from N or S.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 14, and an LCDR3 sequence comprising or consisting of a sequence of SEQ ID NO; 24 or 26, wherein X4 is selected from A or G, and X5 is selected from D or E.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19; and the VL region comprises an LCDR1 sequence comprising or consisting of a sequence of SEQ ID NO; 22 or 27, an LCDR2 sequence comprising or consisting of a sequence of SEQ ID NO; 23. 25, or 28, and LCDR3 sequence comprising or consisting of a sequence of SEQ ID NO; 15, wherein X6 is present or absent, and if present, is F.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19, and the VL region comprises an LCDR1 sequence comprising or consisting of a sequence of SEQ ID NO; 22, an LCDR2 sequence comprising or consisting of a sequence of SEQ ID NO; 16, and an LCDR3 sequence comprising or consisting of a sequence of SEQ ID NO; 15, wherein X5 is selected from D or E and X6 is present or absent, and if present, is F.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 23, and an LCDR3 sequence comprising or consisting of a sequence of SEQ ID NO; 24.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 25, and an LCDR3 sequence comprising or consisting of a sequence of SEQ ID NO; 26.

In some instances, the anti-transferrin receptor antibody comprises a VH region and a VL region, in which the VH region comprises an HCDR1 sequence comprising or consisting of a sequence of SEQ ID NO; 17, an HCDR2 sequence comprising or consisting of a sequence of SEQ ID NO; 21, and an HCDR3 sequence comprising or consisting of a sequence of SEQ ID NO; 19; and the VL region comprises an LCDR1 sequence comprising or consisting of a sequence of SEQ ID NO; 27, an LCDR2 sequence comprising or consisting of a sequence of SEQ ID NO; 28, and an LCDR3 sequence comprising or consisting of a sequence of SEQ ID NO; 26.

In some instances, the anti-transferrin receptor antibody comprises a VH region and a VL region, in which the VH region comprises an HCDR1 sequence an HCDR1 sequence comprising or consisting of NYWMH (SEQ ID NO; 67), an HCDR2 sequence comprising or consisting of a sequence of SEQ ID NO; 20, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 23, and an LCDR3 sequence comprising or consisting of a sequence of SEQ ID NO; 24.

In some aspects, the anti-transferrin receptor antibody comprises a VH region and a VL region in which the sequence of the VH region comprises or consists of a sequence with about 80%, about 85%, about 90%, about 95%, about 96%, about 97%, about 98%, about 99%, or about 100% sequence identity to a sequence selected from SEQ ID NOs; 29-33 and the sequence of the VL region comprises or consisting of a sequence with about 80%, about 85%, about 90%, about 95%, about 96%, about 97%, about 98%, about 99%, or about 100% sequence identity to a sequence selected from SEQ ID NOs; 34-38. In some aspects, the anti-transferrin receptor antibody comprises a VH region and a VL region in which the sequence of the VH region comprises or consists of a sequence with at least 80%, at least 85%, at least 90%, at least 95%, at least 96%, at least 97%, at least 98%, at least 99%, or 100% sequence identity to a sequence selected from a sequence of SEQ ID NOs; 29-33 and the sequence of the VL region comprises or consists of a sequence with at least 80%, at least 85%, at least 90%, at least 95%, at least 96%, at least 97%, at least 98%, at least 99%, or 100% sequence identity to a sequence selected from a sequence of SEQ ID NOs; 34-38. In some aspects, the anti-transferrin receptor antibody comprises a VH region and a VL region in which the sequence of the VH region comprises or consists of a sequence selected from a sequence of SEQ ID NOs; 29-33 and the sequence of the VL region comprises or consists of a sequence selected from a sequence of SEQ ID NOs; 34-38.

In some aspects, the VH region comprises or consists of a sequence selected from SEQ ID NOs; 29-33 (Table 6) and the VL region comprises or consists of a sequence selected from SEQ ID NOs; 34-38 (Table 7). The underlined regions in Table 6 and Table 7 denote the respective CDR1, CDR2, or CDR3 sequence.

TABLE 6

| | | SEQ ID |
|---|---|---|
| NAME | VH SEQUENCE | NO: |
| 13E4_VH1 | QVQLVQSGAEVKKPGASVKVSCKASGYTFTNYWMHWVRQAPGQ | 29 |
| | GLEWMGEINPINGRSNYAQKFQGRVTLTVDTSISTAYMELSRLRSD | |
| | DTAVYYCARGTRAMHYWGQGTLVTVSS | |
| 13E4_VH2 | QVQLVQSGAEVKKPGASVKVSCKASGYTFTNYWMHWVRQAPGQ | 30 |
| | GLEWIGEINPINGRSNYAEKFQGRVTLTVDTSSSTAYMELSRLRSDD | |
| | TAVYYCARGTRAMHYWGQGTLVTVSS | |
| 13E4_VH3 | QVQLVQSGAEVKKPGASVKVSCKASGYTFTNYWMHWVRQAPGQ | 31 |
| | GLEWMGEINPIQGRSNYAEKFQGRVTLTVDTSSSTAYMELSSLRSE | |
| | DTATYYCARGTRAMHYWGQGTLVTVSS | |
| 13E4_VH4 | QVQLVQSGAEVKKPGASVKVSCKASGYTFTNYWMHWVRQAPGQ | 32 |
| | GLEWMGEINPINGRSNYAEKFQGRVTLTVDTSSSTAYMELSSLRSE | |
| | DTATYYCARGTRAMHYWGQGTLVTVSS | |
| 13E4_VH | QVQLQQPGAELVKPGASVKLSCKASGYTFTNYWMHWVKQRPGQG | 33 |
| | LEWIGEINPINGRSNYGERFKTKATLTVDKSSSTAYMQLSSLTSEDS | |
| | AVYYCARGTRAMHYWGQGTSVTVSS | |

TABLE 7

| | | SEQ ID |
|---|---|---|
| NAME | VL SEQUENCE | NO: |
| 13E4_VL1 | DIQMTQSPSSLSASVGDRVTITCRTSENIYNNLAWYQQKPGKSPKLL | 34 |
| | IYAATNLADGVPSRFSGSGSGTDYTLTISSLQPEDFATYYCQHFWGT | |
| | PLTFGGGTKVEIK | |
| 13E4_VL2 | DIQMTQSPSSLSASVGDRVTITCRTSENIYNNLAWYQQKPGKAPKLL | 35 |
| | IYAATNLADGVPSRFSGSGSGTDYTLTISSLQPEDFATYYCQHFWGT | |
| | PLTFGGGTKVEIK | |
| 13E4_VL3 | DIQMTQSPSSLSASVGDRVTITCRTSENIYNNLAWYQQKPGKAPKLL | 36 |
| | IYAATNLAEGVPSRFSGSGSGTDYTLTISSLQPEDFATYYCQHFWGT | |
| | PLTFGGGTKVEIK | |
| 13E4_VL4 | DIQMTQSPSSLSASVGDRVTITCRTSENIYSNLAWYQQKPGKAPKLL | 37 |
| | IYAGTNLADGVPSRFSGSGSGTDYTLTISSLQPEDFANYYCQHFWGT | |
| | PLTFGGGTKVEIK | |
| 13E4_VL | DIQMTQSPASLSVSVGETVTITCRTSENIYNNLAWYQQKQGKSPQLL | 38 |
| | VYAATNLADGVPSRFSGSGSGTQYSLKINSLQSEDFGNYYCQHFWG | |
| | TPLTFGAGTKLELK | |

In some aspects, the anti-transferrin receptor antibody comprises a VH region and a VL region as illustrated in Table 8.

TABLE 8

| | 13E4_VH1 | 13E4_VH2 | 13E4_VH3 | 13E4_VH4 |
|---|---|---|---|---|
| | (SEQ ID NO: 29) | (SEQ ID NO: 30) | (SEQ ID NO: 31) | (SEQ ID NO: 32) |
| 13E4_VL1 | SEQ ID NO: 29 | SEQ ID NO: 30 | SEQ ID NO: 31 | SEQ ID NO: 32 |
| (SEQ ID NO: 34) | + SEQ ID NO: 34 | + SEQ ID NO: 34 | + SEQ ID NO: 34 | + SEQ ID NO: 34 |
| 13E4_VL2 | SEQ ID NO: 29 | SEQ ID NO: 30 | SEQ ID NO: 31 | SEQ ID NO: 32 |
| (SEQ ID NO: 35) | + SEQ ID NO: 35 | + SEQ ID NO: 35 | + SEQ ID NO: 35 | + SEQ ID NO: 35 |
| 13E4_VL3 | SEQ ID NO: 29 | SEQ ID NO: 30 | SEQ ID NO: 31 | SEQ ID NO: 32 |
| (SEQ ID NO: 36) | + SEQ ID NO: 36 | + SEQ ID NO: 36 | + SEQ ID NO: 36 | + SEQ ID NO: 36 |
| 13E4_VL4 | SEQ ID NO: 29 | SEQ ID NO: 30 | SEQ ID NO: 31 | SEQ ID NO: 32 |
| (SEQ ID NO: 37) | + SEQ ID NO: 37 | + SEQ ID NO: 37 | + SEQ ID NO: 37 | + SEQ ID NO: 37 |

In some aspects, an anti-transferrin receptor antibody described herein comprises an IgG framework, an IgA framework, an IgE framework, or an IgM framework. In some instances, the anti-transferrin receptor antibody comprises an IgG framework (e.g., IgG1, IgG2, IgG3, or IgG4). In some cases, the anti-transferrin receptor antibody comprises an IgGI framework. In some cases, the anti-transferrin receptor antibody comprises an IgG2 (e.g., an IgG2a or IgG2b) framework. In some cases, the anti-transferrin receptor antibody comprises an IgG2a framework. In some cases, the anti-transferrin receptor antibody comprises an IgG2b framework. In some cases, the anti-transferrin receptor antibody comprises an IgG3 framework. In some cases, the anti-transferrin receptor antibody comprises an IgG4 framework.

In some cases, an anti-transferrin receptor antibody comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional instances, the one or more mutations are to modulate glycosylation.

In some aspects, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some instances, the Fc region comprises a mutation at residue position L234, L235, D265, N21, K46, L52, or P53, or a combination thereof. In some instances, the mutations comprise L234 and L235 in combination with a mutation at residue position K46, L52, or P53. In some cases, the Fc region comprises mutations at L234, L235, and K46. In some cases, the Fc region comprises mutations at L234, L235, and L52. In some cases, the Fc region comprises mutations at L234, L235, and P53. In some cases, the Fc region comprises mutations at D265 and N21. In some cases, the residue position is in reference to IgGI.

In some instances, the Fc region comprises L234A, L235A, D265A, N21G, K46G, L52R, or P53G, or a combination thereof. In some instances, the Fc region comprises L234A and L235A in combination with K46G, L52R, or P53G. In some cases, the Fc region comprises L234A, L235A, and K46G. In some cases, the Fc region comprises L234A, L235A, and L52R. In some cases, the Fc region comprises L234A, L235A, and P53G. In some cases, the Fc region comprises D265A and N21G. In some cases, the residue position is in reference to IgG1.

In some instances, the Fc region comprises a mutation at residue position L235, L236, D265, N21, K46, L52, or P53, or a combination of the mutations. In some instances, the Fc region comprises mutations at L235 and L236. In some instances, the Fc region comprises mutations at L235 and L236 in combination with a mutation at residue position K46, L52, or P53. In some cases, the Fc region comprises mutations at L235, L236, and K46. In some cases, the Fc region comprises mutations at L235, L236, and L52. In some cases, the Fc region comprises mutations at L235, L236, and P53. In some cases, the Fc region comprises mutations at D265 and N21. In some cases, the residue position is in reference to IgG2b.

In some aspects, the Fc region comprises L235A, L236A, D265A, N21G, K46G. L52R, or P53G, or a combination thereof. In some instances, the Fc region comprises L235A and L236A. In some instances, the Fc region comprises L235A and L236A in combination with K46G, L52R, or P53G. In some cases, the Fc region comprises L235A, L236A, and K46G. In some cases, the Fc region comprises L235A, L236A, and L52R. In some cases, the Fc region comprises L235A, L236A, and P53G. In some cases, the Fc region comprises D265A and N21G. In some cases, the residue position is in reference to IgG2b.

In some aspects, the Fc region comprises a mutation at residue position L233, L234, D264, N20, K45, L51, or P52, wherein the residues correspond to positions 233, 234, 264, 327, 20, 45, 51, and 52 of SEQ ID NO; 39. In some instances, the Fc region comprises mutations at L233 and L234. In some instances, the Fc region comprises mutations at L233 and L234 in combination with L327. In some instances, the Fc region comprises mutations at L233 and L234 in combination with a mutation at residue position K45, L51, or P52. In some cases, the Fc region comprises mutations at L233, L234, and K45. In some cases, the Fc region comprises mutations at L233, L234, and L51. In some cases, the Fc region comprises mutations at L233, L234, and K45. In some cases, the Fc region comprises mutations at L233, L234, and P52. In some instances, the Fc region comprises mutations at D264 and N20. In some cases, equivalent positions to residue L233, L234, D264, N20, K45, L51, or P52 in an IgG1, IgG2. IgG3, or IgG4 framework are contemplated. In some cases, mutations to a residue that corresponds to residue L233, L234, D264, N20, K45, L51, or P52 of SEQ ID NO; 39 in an IgG1, IgG2, or IgG4 framework are also contemplated.

In some aspects, the Fc region comprises L233A, L234A, L327R, D264A, N20G, K45G, L51R, or P52G, wherein the residues correspond to positions 233, 234, 327, 264, 20, 45, 51, and 52 of SEQ ID NO; 39. In some instances, the Fc region comprises L233A and L234A. In some instances, the Fc region comprises L233A and L234A in combination with L327R. In some instances, the Fc region comprises L233A and L234A in combination with K45G, L5IR, or P52G. In some cases, the Fc region comprises L233A, L234A, and K45G. In some cases, the Fc region comprises L233A, L234A, and L51R. In some cases, the Fc region comprises L233A. L234A, and K45G. In some cases, the Fc region comprises L233A, L234A, and P52G. In some instances, the Fc region comprises D264A and N20G.

In some aspects, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68 (10); 3863-72: Idusogie et al., 2001 J Immunol, 166 (4); 2571-5; Moore et al., 2010 mAbs, 2 (2); 181-189; Lazar et al., 2006 PNAS, 103 (11); 4005-4010, Shields et al., 2001 JBC, 276 (9); 6591-6604: Stavenhagen et al., 2007 Cancer Res, 67 (18); 8882-8890; Stavenhagen et al., 2008 Advan. Enzyme Regul., 48:152-164: Alegre et al, 1992 J Immunol, 148:3461-3468; Reviewed in Kaneko and Niwa, 2011 Biodrugs, 25 (1); 1-11.

### Substituts Specification-Clean

In some aspects, an anti-transferrin receptor antibody described herein is a full-length antibody, comprising a heavy chain (HC) and a light chain (LC). In some cases, the heavy chain (HC) comprises a sequence selected from Table 9. In some cases, the light chain (LC) comprises a sequence selected from Table 10. The underlined region denotes the respective CDRs.

TABLE 9

| | | SEQ ID |
|---|---|---|
| NAME | HC SEQUENCE | NO: |
| 13E4_VH1 | QVQLVQSGAEVKKPGASVKVSCKASGYTFTNYWMHWVRQAPG | 39 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1_a | QVQLVQSGAEVKKPGASVKVSCKASGYTFTNYWMHWVRQAPG | 40 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1_b | QVQLVQSGAEVKKPGASVKVSCKASGYTFTNYWMHWVRQAPG | 41 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCGVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1_c | QVQLVQSGAEVKKPGASVKVSCKASGYTFTNYWMHWVRQAPG | 42 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKARPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1_d | QVQLVQSGAEVKKPGASVKVSCKASGYTFTNYWMHWVRQAPG | 43 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALGAPIEKTISKAKGQPREPQVYT | |
| | LPPSREEMTKNQVSLTCLVKGFYPSDIAVEWESNGQPENNYKTT | |
| | PPVLDSDGSFFLYSKLTVDKSRWQQGNVFSCSVMHEALHNHYT | |
| | QKSLSLSPG | |
| 13E4_VH1_e | QVQLVQSGAEVKKPGASVKVSCKASGYTFTNYWMHWVRQAPG | 44 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPELLGGPSVFLFPPKPKDTLMISRTPEVTCVVV | |
| | AVSHEDPEVKFNWYVDGVEVHNAKTKPREEQYGSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH2 | QVQLVQSGAEVKKPGASVKVSCKASGYTFTNYWMHWVRQAPG | 45 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2_a | QVQLVQSGAEVKKPGASVKVSCKASGYTFTNYWMHWVRQAPG | 46 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2_b | QVQLVQSGAEVKKPGASVKVSCKASGYTFTNYWMHWVRQAPG | 47 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCGVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2_c | QVQLVQSGAEVKKPGASVKVSCKASGYTFTNYWMHWVRQAPG | 48 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKARPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2_d | QVQLVQSGAEVKKPGASVKVSCKASGYTFTNYWMHWVRQAPG | 49 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALGAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2_e | QVQLVQSGAEVKKPGASVKVSCKASGYTFTNYWMHWVRQAPG | 50 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVA | |
| | VSHEDPEVKFNWYVDGVEVHNAKTKPREEQYGSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3 | QVQLVQSGAEVKKPGASVKVSCKASGYTFTNYWMHWVRQAPG | 51 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_a | QVQLVQSGAEVKKPGASVKVSCKASGYTFTNYWMHWVRQAPG | 52 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_b | QVQLVQSGAEVKKPGASVKVSCKASGYTFTNYWMHWVRQAPG | 53 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCGVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_c | QVQLVQSGAEVKKPGASVKVSCKASGYTFTNYWMHWVRQAPG | 54 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKARPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_d | QVQLVQSGAEVKKPGASVKVSCKASGYTFTNYWMHWVRQAPG | 55 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALGAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_e | QVQLVQSGAEVKKPGASVKVSCKASGYTFTNYWMHWVRQAPG | 56 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVA | |
| | VSHEDPEVKFNWYVDGVEVHNAKTKPREEQYGSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4 | QVQLVQSGAEVKKPGASVKVSCKASGYTFTNYWMHWVRQAPG | 57 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_a | QVQLVQSGAEVKKPGASVKVSCKASGYTFTNYWMHWVRQAPG | 58 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_b | QVQLVQSGAEVKKPGASVKVSCKASGYTFTNYWMHWVRQAPG | 59 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCGVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_c | QVQLVQSGAEVKKPGASVKVSCKASGYTFTNYWMHWVRQAPG | 60 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKARPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_d | QVQLVQSGAEVKKPGASVKVSCKASGYTFTNYWMHWVRQAPG | 61 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALGAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_e | QVQLVQSGAEVKKPGASVKVSCKASGYTFTNYWMHWVRQAPG | 62 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVA | |
| | VSHEDPEVKFNWYVDGVEVHNAKTKPREEQYGSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |

TABLE 10

| | | SEQ ID |
|---|---|---|
| NAME | LC SEQUENCE | NO: |
| 13E4_VL1 | DIQMTQSPSSLSASVGDRVTITCRTSENIYNNLAWYQQKPGKSPK | 63 |
| | LLIYAATNLADGVPSRFSGSGSGTDYTLTISSLQPEDFATYYCQHF | |
| | WGTPLTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| 13E4_VL2 | DIQMTQSPSSLSASVGDRVTITCRTSENIYNNLAWYQQKPGKAPK | 64 |
| | LLIYAATNLADGVPSRFSGSGSGTDYTLTISSLQPEDFATYYCQHF | |
| | WGTPLTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| 13E4_VL3 | DIQMTQSPSSLSASVGDRVTITCRTSENIYNNLAWYQQKPGKAPK | 65 |
| | LLIYAATNLAEGVPSRFSGSGSGTDYTLTISSLQPEDFATYYCQHF | |
| | WGTPLTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| 13E4_VL4 | DIQMTQSPSSLSASVGDRVTITCRTSENIYSNLAWYQQKPGKAPK | 66 |
| | LLIYAGTNLADGVPSRFSGSGSGTDYTLTISSLQPEDFANYYCQH | |
| | FWGTPLTFGGGTKVEIKRTVAAPSVFIFPPSDEQLKSGTASVVCL | |
| | LNNFYPREAKVQWKVDNALQSGNSQESVTEQDSKDSTYSLSSTL | |
| | TLSKADYEKHKVYACEVTHQGLSSPVTKSFNRGEC | |

In some aspects, an anti-transferrin receptor antibody described herein has an improved serum half-life compared to a reference anti-transferrin receptor antibody. In some instances, the improved serum half-life is at least 30 minutes, 1 hour, 1.5 hours, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than the reference anti-transferrin receptor antibody.

In some aspects, an antibody or antigen binding fragment thereof is further modified using conventional techniques known in the art, for example, by using amino acid deletion, insertion, substitution, addition, and/or by recombination and/or any other modification (e.g., posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor. In some instances, such modifications can be introduced in the nucleic acid sequence underlying the amino acid sequence of an antibody or its binding fragment using any conventional methods in the art.

In some instances, an antibody or antigen binding fragment thereof further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such embodiments, the bi-specific single chain antibody construct is tandem to a bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some aspects, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to whether the antibody or its binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g, due to the induction of a change of the conformation of the antigen, an oligomerization of the antigen, etc. In further aspects, the binding is exemplified by the specificity of a “key-lock-principle”. Thus in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well as in a simple binding of the site to the antigen.

In some instances, specific interaction further refers to a reduced cross-reactivity of the antibody or its binding fragment or a reduced off-target effect. For example, the antibody or antigen binding fragment thereof that binds to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

Thus, in some instances, a polynucleic acid molecule conjugate comprises a polynucleic acid molecule (e.g., PMO molecule) comprising or consisting of a sequence with at least 80%, at least 85%, at least 90%, at least 95%, at least 99%, or 100% sequence identity to a nucleic acid sequence selected from SEQ ID NOs; 100-169, and an anti-transferrin receptor antibody or antigen binding fragment thereof conjugated to the polynucleic acid such that the polynucleic acid molecule conjugate induces exon skipping of the pre-mRNA of the DMD gene.

In certain aspects, a polynucleic acid molecule conjugate comprises an anti-transferrin receptor antibody or antigen binding fragment thereof conjugated to a polynucleic acid molecule (e.g., PMO molecule) that hybridizes to a target region of a pre-mRNA transcript of the DMD gene, and the polynucleic acid molecule having a sense strand comprising or consisting of a nucleic acid sequence with at least 80%, at least 85%, at least 90%, at least 95%, at least 99%, or 100% sequence identity to a nucleic acid sequence selected from SEQ ID NOs; 100-169, and anti-transferrin receptor antibody or antigen binding fragment thereof comprises a variable heavy chain (VH) region and a variable light chain (VL) region, wherein the VH region comprises an HCDR1 sequence comprising or consisting of SEQ ID NO; 17, an HCDR2 sequence comprising or consisting of SEQ ID NO; 20, and an HCDR3 sequence comprising or consisting of SEQ ID NO; 19; and the VL region comprises an LCDR1 sequence comprising or consisting of SEQ ID NO; 22, an LCDR2 sequence comprising or consisting of SEQ ID NO; 23, and an LCDR3 sequence comprising or consisting of SEQ ID NO; 24, and the anti-transferrin receptor antibody or antigen binding fragment thereof and the polynucleic acid molecule is conjugated via a linker comprising 4-(N-maleimidomethyl) cyclohexane-1-amidate (SMCC) or 6-maleimidocaproic acid (MC).

In certain aspects, a polynucleic acid molecule conjugate comprises an anti-transferrin receptor antibody or antigen binding fragment thereof conjugated to a polynucleic acid molecule (e.g., PMO molecule) that hybridizes to a target region of a pre-mRNA transcript of the DMD gene, and the polynucleic acid molecule having a sense strand comprising or consisting of a nucleic acid sequence with at least 80%, at least 85%, at least 90%, at least 95%, at least 99%, or 100% sequence identity to a nucleic acid sequence selected from SEQ ID NOs; 100-169, and anti-transferrin receptor antibody or antigen binding fragment thereof comprises a variable heavy chain (VH) region and a variable light chain (VL) region, wherein the VH region comprises an HCDR1 sequence an HCDR1 sequence comprising or consisting of NYWMH (SEQ ID NO; 67), an HCDR2 sequence comprising or consisting of a sequence of SEQ ID NO: 20, and an HCDR3 sequence comprising or consisting of a sequence of SEQ ID NO; 19; and the VL region comprises an LCDR1 sequence comprising or consisting of a sequence of SEQ ID NO; 22, an LCDR2 sequence comprising or consisting of a sequence of SEQ ID NO; 23, and an LCDR3 sequence comprising or consisting of a sequence of SEQ ID NO; 24 . and the anti-transferrin receptor antibody or antigen binding fragment thereof and the polynucleic acid molecule is conjugated via a linker comprising 4-(N-maleimidomethyl) cyclohexane-1-amidate (SMCC) or 6-maleimidocaproic acid (MC).

In certain aspects, a polynucleic acid molecule conjugate comprises an anti-transferrin receptor antibody or antigen binding fragment thereof conjugated to a polynucleic acid molecule (e.g., PMO molecule) that hybridizes to a sequence of a target region of a pre-mRNA transcript of the DMD gene, and the polynucleic acid molecule comprising or consisting of a nucleic acid sequence with at least 80%, at least 85%, at least 90%, at least 95%, at least 99%, or 100% sequence identity to a nucleic acid sequence selected from SEQ ID NOs; 100-169 and the anti-transferrin receptor antibody or antigen binding fragment thereof comprises a variable heavy chain (VH) region and a variable light chain (VL) region, wherein the VH region comprises or consisting of a nucleic acid sequence with at least 80%, 85%, 90%, 95%. 99%, or 100% sequence identity to SEQ ID NO; 30, and wherein the VL region comprises or consisting of a nucleic acid sequence with at least 80%, 85%, 90%, 95%. 99%, or 100% sequence identity to SEQ ID NO; 34, and the anti-transferrin receptor antibody or antigen binding fragment thereof and the polynucleic acid molecule is conjugated via a maleimide linker.

In certain aspects, a polynucleic acid molecule conjugate comprises an anti-transferrin receptor antibody or antigen binding fragment thereof conjugated to a polynucleic acid molecule (e.g., PMO molecule) that hybridizes to a sequence of a target region of a pre-mRNA transcript of the DMD gene, and the polynucleic acid molecule comprising or consisting of a nucleic acid sequence with at least 80%, at least 85%, at least 90%, at least 95%, at least 99%, or 100% sequence identity to a nucleic acid sequence selected from SEQ ID NOs; 100-169, and comprises at least three, four, five, or six consecutive 2′-O-methyl modified nucleotides at the 5′-end and at least two, at least three 2′-F modified nucleotides, and the anti-transferrin receptor antibody or antigen binding fragment thereof comprises a variable heavy chain (VH) region and a variable light chain (VL) region, wherein the VH region comprises or consisting of a sequence with at least 80%, 85%, 90%, 95%. 99%, or 100% sequence identity to SEQ ID NO; 30, and wherein the VL region comprises or consisting of a sequence with at least 80%, 85%, 90%, 95%. 99%, or 100% sequence identity to SEQ ID NO; 34, and the anti-transferrin receptor antibody or antigen binding fragment thereof and the polynucleic acid molecule is conjugated via a maleimide linker.

In certain aspects, a polynucleic acid molecule conjugate comprises an anti-transferrin receptor antibody or antigen binding fragment thereof conjugated to a polynucleic acid molecule (e.g., PMO molecule) that hybridizes to a sequence of a target region of a pre-mRNA transcript of the DMD gene, and the polynucleic acid molecule comprising or consisting of a nucleic acid sequence with at least 80%, at least 85%, at least 90%, at least 95%, at least 99%, or 100% sequence identity to a nucleic acid sequence selected from SEQ ID NOs; 100-169, and comprises at least two, at least three, at least four, or at least five consecutive 2′-O-methyl modified nucleotide at the 3′-end of the polynucleic acid molecule, and comprises at least one, at least two, at least three, at least four 2′-F modified nucleotides, and the anti-transferrin receptor antibody or antigen binding fragment thereof comprises a variable heavy chain (VH) region and a variable light chain (VL) region, wherein the VH region comprises an HCDR1 sequence comprising or consisting of SEQ ID NO; 17 or NYWMH (SEQ ID NO; 67), an HCDR2 sequence comprising or consisting of SEQ ID NO; 20, and an HCDR3 sequence comprising or consisting of SEQ ID NO; 19; and the VL region comprises an LCDR1 sequence comprising or consisting of SEQ ID NO; 22, an LCDR2 sequence comprising or consisting of SEQ ID NO; 23, and an LCDR3 sequence comprising or consisting of SEQ ID NO; 24, and the anti-transferrin receptor antibody or antigen binding fragment thereof and the polynucleic acid molecule is conjugated via a maleimide linker.

In certain aspects, a polynucleic acid molecule conjugate comprises an anti-transferrin receptor antibody or antigen binding fragment thereof conjugated to a polynucleic acid molecule (e.g., PMO molecule) that hybridizes to a sequence of a target region of a pre-mRNA transcript of the DMD gene, and the polynucleic acid molecule comprises or consists of a nucleic acid sequence with at least 80%, at least 85%, at least 90%, at least 95%, at least 99%, or 100% sequence identity to a nucleic acid sequence selected from SEQ ID NOs; 100-169, comprises one or more 2′-O-methyl modified nucleotides at the 5′-end and/or at the 3′-end of the polynucleic acid molecule, and the anti-transferrin receptor antibody or antigen binding fragment thereof comprises a variable heavy chain (VH) region and a variable light chain (VL) region, and the VH region comprises an HCDR1 sequence comprising or consisting of SEQ ID NO; 17, an HCDR2 sequence comprising or consisting of SEQ ID NO; 18, and an HCDR3 sequence comprising or consisting of SEQ ID NO; 19; and the VL region comprises an LCDR1 sequence comprising or consisting of SEQ ID NO; 22, an LCDR2 sequence comprising or consisting of SEQ ID NO; 3, and an LCDR3 sequence comprising or consisting of SEQ ID NO: 24, and the anti-transferrin receptor antibody or antigen binding fragment thereof and the polynucleic acid molecule is conjugated via a maleimide linker.

In certain aspects, a polynucleic acid molecule conjugate comprises an anti-transferrin receptor antibody or antigen binding fragment thereof conjugated to a polynucleic acid molecule (e.g., PMO molecule) that hybridizes to a target sequence of a pre-mRNA transcript of the DMD gene, and the polynucleic acid molecule comprising or consisting of a nucleic acid sequence with at least 80%, at least 85%, at least 90%, at least 95%, at least 99%, or 100% sequence identity to a nucleic acid sequence selected from SEQ ID NOs; 100-169, and the anti-transferrin receptor antibody or antigen binding fragment thereof comprises a variable heavy chain (VH) region and a variable light chain (VL) region, and the VH region comprises or consists of a sequence with at least 80%, at least 85%, at least 90%, at least 95%, at least 99%, or 100% sequence identity to one of SEQ ID NOs; 29-33, and wherein the VL region comprises or consists of a sequence with at least 80%, at least 85%, at least 90%, at least 95%, at least 99%, or 100% sequence identity to one of SEQ ID NOs; 34-38, and the anti-transferrin receptor antibody or antigen binding fragment thereof and the polynucleic acid molecule is conjugated via a 6-Amino-1-hexanol linker. In some instances, the polynucleic acid molecule comprises at least one or more 2′-modified nucleotides. In some instances, the polynucleic acid molecule comprises at least five consecutive 2′-O-methyl modified nucleotide at the 3′-end. In some instances, the polynucleic acid molecule comprises at least three or at least four 2′-F modified nucleotides, wherein any two of the at least three or at least four 2-F modified nucleotides are not consecutive.

In some aspects, the antibody or antigen binding fragment thereof is conjugated to any of the PMO molecules disclosed herein non-specifically. In some instances, the antibody or antigen binding fragment thereof is conjugated to any of the PMO molecules disclosed herein via a lysine residue or a cysteine residue, in a non-site specific manner. In some instances, the antibody or antigen binding fragment thereof is conjugated to any of the PMO molecules disclosed herein via a lysine residue in a non-site specific manner. In some cases, the antibody or antigen binding fragment thereof is conjugated to any of the PMO molecules disclosed herein via a cysteine residue in a non-site specific manner.

In some aspects, the antibody or antigen binding fragment thereof is conjugated to any of the PMO molecules disclosed herein in a site-specific manner. In some instances, the antibody or antigen binding fragment thereof is conjugated to any of the PMO molecules disclosed herein through a lysine residue, a cysteine residue, at the 5′-terminus, at the 3′-terminus, an unnatural amino acid, or an enzyme-modified or enzyme-catalyzed residue, via a site-specific manner. In some instances, the antibody or antigen binding fragment thereof is conjugated to any of the PMO molecules disclosed herein through a lysine residue via a site-specific manner. In some instances, the antibody or antigen binding fragment thereof is conjugated to any of the PMO molecules disclosed herein through a cysteine residue via a site-specific manner. In some instances, the antibody or antigen binding fragment thereof is conjugated to any of the PMO molecules disclosed herein at the 5′-terminus via a site-specific manner. In some instances, the antibody or antigen binding fragment thereof is conjugated to any of the PMO molecules disclosed herein at the 3′-terminus via a site-specific manner. In some instances, the antibody or antigen binding fragment thereof is conjugated to any of the PMO molecules disclosed herein through an unnatural amino acid via a site-specific manner. In some instances, the antibody or antigen binding fragment thereof is conjugated to any of the PMO molecules disclosed herein through an enzyme-modified or enzyme-catalyzed residue via a site-specific manner.

In some aspects, one or more PMO molecule is conjugated to any of the antibodies or antigen binding fragments thereof disclosed herein. In some instances, about 1, 2, 3, 4, 5, 6, 7, 8, 9, 10, 11, 12, 13, 14. 15, 16, or more PMO molecules are conjugated to an antibody or antigen binding fragment thereof. In some instances, about 1 PMO molecule is conjugated to one antibody or antigen binding fragment thereof. In some instances, about 2 PMO molecules are conjugated to one antibody or antigen binding fragment thereof. In some instances, about 3 PMO molecules are conjugated to one antibody or antigen binding fragment thereof. In some instances, about 4 PMO molecules are conjugated to one. In some instances, about 5 PMO molecules are conjugated to one antibody or antigen binding fragment thereof. In some instances, about 6 PMO molecules are conjugated to one antibody or antigen binding fragment thereof. In some instances, about 7 PMO molecules are conjugated to one antibody or antigen binding fragment thereof. In some instances, about 8 PMO molecules are conjugated to one antibody or antigen binding fragment thereof. In some instances, about 9 PMO molecules are conjugated to one antibody or antigen binding fragment thereof. In some instances, about 10) PMO molecules are conjugated to one antibody or antigen binding fragment thereof.

In some aspects, at least 1, 2, 3, 4, 5, 6, 7, 8, 9, 10, 11, 12, 13, 14, 15, 16, 17, 18, 19, or 20 ASO or PMO molecules are conjugated to an antibody or antigen binding fragment thereof. In some instances, at least 1 ASO or PMO molecule is conjugated to one antibody or antigen binding fragment thereof. In some instances, at least 2 ASO or PMO molecules are conjugated to one antibody or antigen binding fragment thereof. In some instances, at least 3 ASO or PMO molecules are conjugated to one antibody or antigen binding fragment thereof. In some instances, at least 4 ASO or PMO molecules are conjugated to one. In some instances, at least 5 ASO or PMO molecules are conjugated to one antibody or antigen binding fragment thereof. In some instances, at least 6 ASO or PMO molecules are conjugated to one antibody or antigen binding fragment thereof. In some instances, at least 7 ASO or PMO molecules are conjugated to one antibody or antigen binding fragment thereof. In some instances, at least 8 ASO or PMO molecules are conjugated to one antibody or antigen binding fragment thereof.

In some instances, from about 1 to about 16 ASO or PMO molecules are conjugated to an antibody or antigen binding fragment thereof. In some instances, from about 2 to about 15 ASO or PMO molecules are conjugated to an antibody or antigen binding fragment thereof. In some instances, from about 3 to about 14 ASO or PMO molecules are conjugated to an antibody or antigen binding fragment thereof. In some instances, from about 4 to about 13 ASO or PMO molecules are conjugated to an antibody or antigen binding fragment thereof. In some instances, from about 5 to about 12 ASO or PMO molecules are conjugated to an antibody or antigen binding fragment thereof. In some instances, from about 6 to about 11 ASO or PMO molecules are conjugated to an antibody or antigen binding fragment thereof. In some instances, from about 7 to about 10 ASO or PMO molecules are conjugated to an antibody or antigen binding fragment thereof. In some instances, from about 8 to about 9 ASO or PMO molecules are conjugated to an antibody or antigen binding fragment thereof.

In some aspects, an average of one or more ASO or PMO molecule is conjugated to an antibody or antigen binding fragment thereof. In some instances, an average of about 1, 2, 3, 4, 5, 6, 7, 8, 9, 10, 11, 12, 13, 14, 15, 16, or more ASO or PMO molecules are conjugated to an antibody or antigen binding fragment thereof. In some instances, an average of about 1 ASO or PMO molecule is conjugated to one antibody or antigen binding fragment thereof. In some instances, an average of about 2 ASO or PMO molecules are conjugated to one antibody or antigen binding fragment thereof. In some instances, an average of about 3 ASO or PMO molecules are conjugated to one antibody or antigen binding fragment thereof. In some instances, an average of about 4 ASO or PMO molecules are conjugated to one antibody or antigen binding fragment thereof. In some instances, an average of about 5 ASO or PMO molecules are conjugated to one antibody or antigen binding fragment thereof. In some instances, an average of about 6 ASO or PMO molecules are conjugated to one antibody or antigen binding fragment thereof. In some instances, an average of about 7 ASO or PMO molecules are conjugated to one antibody or antigen binding fragment thereof. In some instances, an average of about 8 ASO or PMO molecules are conjugated to one antibody or antigen binding fragment thereof.

In some instances, an average of at least 1, 2, 3, 4, 5, 6, 7, 8, 9, 10, 11, 12, 13, 14, 15, 16, or 17 ASO or PMO molecules are conjugated to an antibody or antigen binding fragment thereof. In some instances, an average of at least 1 ASO or PMO molecule is conjugated to one antibody or antigen binding fragment thereof. In some instances, an average of at least 2 ASO or PMO molecules are conjugated to one antibody or antigen binding fragment thereof. In some instances, an average of at least 3 ASO or PMO molecules are conjugated to one antibody or antigen binding fragment thereof. In some instances, an average of at least 4 ASO or PMO molecules are conjugated to one ASO or antibody or antigen binding fragment thereof. In some instances, an average of at least 5 PMO molecules are conjugated to one antibody or antigen binding fragment thereof. In some instances, an average of at least 6 ASO or PMO molecules are conjugated to one antibody or antigen binding fragment thereof. In some instances, an average of at least 7 ASO or PMO molecules are conjugated to one antibody or antigen binding fragment thereof. In some instances, an average of at least 8 ASO or PMO molecules are conjugated to one antibody or antigen binding fragment thereof.

In some aspects, the number of ASO or PMO molecule(s) conjugated to an antibody forms a ratio. In some instances, the ratio is referred to as a DAR (drug-to-antibody) ratio, in which the drug as referred to herein is the ASO or PMO molecule. In some instances, the DAR of the ASO or PMO molecule to antibody is about 1, 2, 3, 4, 5, 6, 7, 8, 9, 10, 11, 12, 13, 14, 15, 16, or greater. In some instances, the DAR of the PMO molecule to antibody is about 1 or greater. In some instances, the DAR of the PMO molecule to antibody is about 2 or greater. In some instances, the DAR of the PMO molecule to antibody is about 3 or greater. In some instances, the DAR of PMO molecule to antibody is about 4 or greater. In some instances, the DAR of the PMO molecules to antibody is about 5 or greater. In some instances, the DAR of the PMO molecule to antibody is about 6 or greater. In some instances, the DAR of the PMO molecule to antibody is about 7 or greater. In some instances, the DAR of the PMO molecule to antibody is about 8 or greater.

In some aspects, the average number of ASO or PMO molecules conjugated to an antibody forms an average ratio. In some instances, the average ratio is referred to as an average DAR (drug-to-antibody) ratio, in which the drug as referred to herein is the PMO molecule. In some instances, the average DAR of the ASO or PMO molecule to antibody is about 1, 2, 3, 4, 5, 6, 7, 8, 9, 10, 11, 12, 13, 14, 15, 16, or greater. In some instances, the average DAR of the PMO molecule to antibody is about 1 or greater. In some instances, the average DAR of the PMO molecule to antibody is about 2 or greater. In some instances, the average DAR of the PMO molecule to antibody is about 3 or greater. In some instances, the average DAR of PMO molecule to antibody is about 4 or greater. In some instances, the average DAR of the PMO molecules to antibody is about 5 or greater. In some instances, the average DAR of the PMO molecule to antibody is about 6 or greater. In some instances, the average DAR of the PMO molecule to antibody is about 7 or greater. In some instances, the average DAR of the PMO molecule to antibody is about 8 or greater.

In some aspects, the average number of ASO or PMO molecules conjugated to an antibody forms an average ratio. In some instances, the average ratio is referred to as an average DAR (drug-to-antibody) ratio, in which the drug as referred to herein is the PMO molecule. In some instances, the average DAR of the PMO molecule to antibody is in the range of 1.0-2.0. 2.0-3.0. 3.0-4.0. 4.0-5.0. 5.0-6.0. 6.0-7.0. 7.0-8.0. 8.0-9.0. 9.0-10.0. 10.0-11.0. 11.0-12.0. 12.0-13.0. 13.0-14.0. 14.0-15.0. 15.0-16.0, or 16.0-17.0. In some instances, the average DAR of the PMO molecule to antibody is in the range of 1.0-2.0. In some instances, the average DAR of the PMO molecule to antibody is in the range of 2.0-3.0. In some instances, the average DAR of the PMO molecule to antibody is in the range of 3.0-4.0. In some instances, the average DAR of PMO molecule to antibody is in the range of 4.0-5.0. In some instances, the average DAR of the PMO molecules to antibody is in the range of 5.0-6.0. In some instances, the average DAR of the PMO molecule to antibody is in the range of 6.0-7.0. In some instances, the average DAR of the PMO molecule to antibody is in the range of 7.0-8.0.

In some aspects, the average number of ASO or PMO molecules conjugated to an antibody forms an average ratio. In some instances, the average ratio is referred to as an average DAR (drug-to-antibody) ratio, in which the drug as referred to herein is the ASO or PMO molecule. In some instances, the average DAR of the ASO or PMO molecule to antibody is in the range of 1.5-2.5. 2.5-3.5. 3.5-4.5, 4.5-5.5, 5.5-6.5, 6.5-7.5, 7.5-8.5, 8.5-9.5, 9.5-10.5, 10.5-11.5, 11.5-12.5. 12.5-13.5, 13.5-14.5. 14.5-15.5, 15.5-16.5, or 16.5-17.5. In some instances, the average DAR of the PMO molecule to antibody is in the range of 1.5-2.5. In some instances, the average DAR of the PMO molecule to antibody is in the range of 2.5-3.5. In some instances, the average DAR of the PMO molecule to antibody is in the range of 3.5-4.5. In some instances, the average DAR of PMO molecule to antibody is in the range of 4.5-5.5. In some instances, the average DAR of the PMO molecules to antibody is in the range of 5.5-6.5. In some instances, the average DAR of the PMO molecule to antibody is in the range of 6.5-7.5. In some instances, the average DAR of the PMO molecule to antibody is in the range of 7.5-8.5. In some instances, the average DAR of the PMO molecule to antibody is in the range of 8.5-9.5. In some instances, the average DAR of the PMO molecule to antibody is in the range of 9.5-10.5.

### Conjugation Chemistry

In some aspects, the polynucleic acid molecule (e.g., ASO or PMO) disclosed herein is conjugated to an antibody (e.g., the antibody disclosed herein). In some instances, the antibody comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of antibody also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the polynucleic acid molecule is further conjugated to a polymer, and optionally an endosomolytic moiety.

In some aspects, the polynucleic acid molecule is conjugated to the antibody by a chemical ligation process. In some instances, the polynucleic acid molecule is conjugated to the antibody by a native ligation. In some instances, the conjugation is as described in: Dawson, et al. “Synthesis of proteins by native chemical ligation.” Science 1994. 266. 776-779: Dawson, et al. “Modulation of Reactivity in Native Chemical Ligation through the Use of Thiol Additives.” J. Am. Chem. Soc. 1997. 119. 4325-4329; Hackeng, et al. “Protein synthesis by native chemical ligation: Expanded scope by using straightforward methodology . . . ” Proc. Natl. Acad. Sci. USA 1999. 96. 10068-10073; or Wu, et al. “Building complex glycopeptides: Development of a cysteine-free native chemical ligation protocol.” Angew. Chem. Int. Ed. 2006. 45. 4116-4125. In some instances, the conjugation is as described in U.S. Pat. No. 8,936,910. In some aspects, the polynucleic acid molecule is conjugated to the antibody either site-specifically or non-specifically via native ligation chemistry.

In some instances, the polynucleic acid molecule is conjugated to the antibody by a site-directed method utilizing a “traceless” coupling technology (Philochem). In some instances, the “traceless” coupling technology utilizes an N-terminal 1,2-aminothiol group on the antibody which is then conjugated with a polynucleic acid molecule containing an aldehyde group. (see Casi et al . . . “Site-specific traceless coupling of potent cytotoxic drugs to recombinant antibodies for pharmacodelivery.” JACS 134 (13); 5887-5892 (2012))

In some instances, the polynucleic acid molecule is conjugated to the antibody by a site-directed method utilizing an unnatural amino acid incorporated into the antibody. In some instances, the unnatural amino acid comprises p-acetylphenylalanine (pAcPhe). In some instances, the keto group of pAcPhe is selectively coupled to an alkoxy-amine derivative conjugating moiety to form an oxime bond. (see Axup et al . . . “Synthesis of site-specific antibody-drug conjugates using unnatural amino acids.” PNAS 109 (40); 16101-16106 (2012)).

In some instances, the polynucleic acid molecule is conjugated to the antibody by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag.” PNAS 106 (9); 3000-3005 (2009); Agarwal, et al . . . “A Pictet-Spengler ligation for protein chemical modification.” PNAS 110 (1); 46-51 (2013))

In some instances, the enzyme-catalyzed process comprises microbial transglutaminase (mTG). In some cases, the polynucleic acid molecule is conjugated to the antibody utilizing a microbial transglutaminase catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (Strop et al . . . “Location matters: site of conjugation modulates stability and pharmacokinetics of antibody drug conjugates.” Chemistry and Biology 20 (2) 161-167 (2013))

In some instances, the polynucleic acid molecule is conjugated to the antibody by a method as described in PCT Publication No. WO2014/140317, which utilizes a sequence-specific transpeptidase.

In some instances, the polynucleic acid molecule is conjugated to the antibody by a method as described in U.S. Patent Publication Nos. 2015/0105539 and 2015/0105540.

### Production of Antibodies or Antigen Binding Fragments Thereof

In some aspects, polypeptides described herein (e.g., antibodies and antigen binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or antigen binding fragment thereof is expressed recombinantly, and the nucleic acid encoding the antibody or antigen binding fragment is assembled from chemically synthesized oligonucleotides (e.g., as described in Kutmeier et al . . . 1994. BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or antigen binding fragment thereof is optionally generated by immunizing an animal, such as a rabbit, to generate polyclonal antibodies or, more preferably, by generating monoclonal antibodies, e.g., as described by Kohler and Milstein (1975. Nature 256:495-497) or, as described by Kozbor et al. (1983. Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibodylibraries (See, e.g., Clackson et al., 1991, Nature 352:624; Hane et al., 1997 Proc. Natl. Acad. Sci. USA 94:4937).

In some aspects, techniques developed for the production of “chimeric antibodies” (Morrison et al., 1984, Proc. Natl. Acad. Sci. 81:851-855; Neuberger et al., 1984, Nature 312:604-608: Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some aspects, techniques described for the production of single chain antibodies (U.S. Pat. No. 4,694,778; Bird, 1988, Science 242:423-42: Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E, coli are also optionally used (Skerra et al., 1988, Science 242:1038-1041).

In some aspects, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue-specific promoter.

In some aspects, a variety of host-expression vector systems is utilized to express an antibody or antigen binding fragment thereof described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E, coli and B, subtilis) transformed with recombinant bacteriophage DNA, plasmid DNA or cosmid DNA expression vectors containing an antibody or its binding fragment coding sequences: yeast (e.g., Saccharomyces Pichia) transformed with recombinant yeast expression vectors containing an antibody or its binding fragment coding sequences: insect cell systems infected with recombinant virus expression vectors (e.g., baculovirus) containing an antibody or its binding fragment coding sequences: plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences: or mammalian cell systems (e.g., COS, CHO. BH. 293. 293T. 3T3 cells) harboring recombinant expression constructs containing promoters derived from the genome of mammalian cells (e.g., metallothionein promoter) or from mammalian viruses (e.g., the adenovirus late promoter: the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977. Cell 11:223), hypoxanthine-guanine phosphoribosyltransferase (Szy balska & Szy balski. 192. Proc. Natl. Acad. Sci. USA 48:202), and adenine phosphoribosyltransferase (Lowy et al . . . 1980. Cell 22:817) genes are employed in tk-, hgprt-or aprt-cells, respectively. Also, antimetabolite resistance are used as the basis of selection for the following genes: dhfr, which confers resistance to methotrexate (Wigler et al., 1980. Proc. Natl. Acad. Sci. USA 77: 357: O'Hare et al . . . 1981. Proc. Natl. Acad. Sci. USA 78:1527): gpt, which confers resistance to mycophenolic acid (Mulligan & Berg. 1981. Proc. Natl. Acad. Sci. USA 78:2072); neo, which confers resistance to the aminoglycoside G-418 (Clinical Pharmacy 12:488-505: Wu and Wu. 1991. Biotherapy 3:87-95: Tolstoshev. 1993. Ann. Rev. Pharmacol. Toxicol. 32:573-596; Mulligan, 1993. Science 260:926-932; and Morgan and Anderson, 1993. Ann. Rev. Biochem. 62:191-217: May. 1993. TIB TECH 11 (5); 155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY: Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.: Colberre-Garapin et al., 1981, J. Mol. Biol. 150:1).

In some instances, the expression levels of an antibody are increased by vector amplification (for a review; see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983. Mol. Cell Biol. 3:257).

In some instances, any method known in the art for purification or analysis of an antibody or antibody conjugates is used, for example, by chromatography (e.g., ion exchange, affinity, particularly by affinity for the specific antigen after Protein A, and sizing column chromatography), centrifugation, differential solubility, or by any other standard technique for the purification of proteins. Exemplary chromatography methods included, but are not limited to, strong anion exchange chromatography, hydrophobic interaction chromatography, size exclusion chromatography, and fast protein liquid chromatography.

### Linkers

In some aspects, a linker described herein is a cleavable linker or a non-cleavable linker. In some instances, the linker is a cleavable linker. In other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C1-C6 alkyl group (e.g., a C5, C4, C3, C2, or C1 alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C5, C4, C3, C2, or CI alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some instances, the non-polymeric linker does not encompass a polymer that is described above. In some instances, the non-polymeric linker does not encompass a polymer encompassed by the polymer moiety C. In some cases, the non-polymeric linker does not encompass a polyalkylene oxide (e.g., PEG). In some cases, the non-polymeric linker does not encompass a PEG.

In some instances, the linker comprises a homobifunctional linker. Exemplary homobifunctional linkers include, but are not limited to. Lomant's reagent dithiobis (succinimidylpropionate) DSP. 3′3′-dithiobis (sulfosuccinimidyl proprionate (DTSSP), disuccinimidyl suberate (DSS), bis (sulfosuccinimidyl) suberate (BS), disuccinimidyl tartrate (DST), disulfosuccinimidyl tartrate (sulfo DST), ethylene glycobis (succinimidylsuccinate) (EGS), disuccinimidyl glutarate (DSG). N,N′-disuccinimidyl carbonate (DSC), dimethyl adipimidate (DMA), dimethyl pimelimidate (DMP), dimethyl suberimidate (DMS), dimethyl-3.3′-dithiobispropionimidate (DTBP). 1,4-di-3′-(2′-pyridyldithio) propionamido) butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene. 4,4′-difluoro-3,3′-dinitrophenylsulfone (DFDNPS), bis-[β-(4-azidosalicy lamido) ethyl|disulfide (BASED), formaldehyde, glutaraldehyde. 1,4-butanediol diglycidyl ether, adipic acid dihydrazide, carbohydrazide, o-toluidine. 3,3′-dimethylbenzidine, benzidine, α,α′-p-diaminodiphenyl, diiodo-p-xylene sulfonic acid. N,N′-ethylene-bis (iodoacetamide), or N,N′-hexamethylene-bis (iodoacetamide).

In some aspects, the linker comprises a heterobifunctional linker. Exemplary heterobifunctional linker include, but are not limited to, amine-reactive and sulfhydryl cross-linkers such as N-succinimidyl 3-(2-pyridyldithio) propionate (sPDP), long-chain N-succinimidyl 3-(2-pyridyldithio) propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio) toluene (sMPT), sulfosuccinimidyl-6-[a-methyl-α-(2-pyridyldithio) toluamido|hexanoate (sulfo-LC-sMPT), succinimidyl-4-(N-maleimidomethyl) cyclohexane-1-carboxylate (sMCC), sulfosuccinimidyl-4-(N-maleimidomethyl) cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs). N-succinimidyl (4-iodoacteyl) aminobenzoate (sIAB), sulfosuccinimidyl (4-iodoacteyl) aminobenzoate (sulfo-sIAB), succinimidyl-4-(p-maleimidophenyl) butyrate (sMPB), sulfosuccinimidyl-4-(p-maleimidophenyl) butyrate (sulfo-sMPB). N-(γ-maleimidobutyry loxy) succinimide ester (GMBs). N-(γ-maleimidobutyryloxy) sulfosuccinimide ester (sulfo-GMBs), succinimidyl 6-((iodoacetyl) amino)hexanoate(sIAX), succinimidyl 6-[6-(((iodoacetyl) amino) hexanoyl) amino|hexanoate (sIAXX), succinimidyl 4-(((iodoacetyl) amino) methyl) cyclohexane-1-carboxylate (SIAC), succinimidyl 6-((((4-iodoacetyl) amino) methyl) cyclohexane-1-carbonyl) amino)hexanoate(sIACX), p-nitrophenyl iodoacetate (NPIA), carbonyl-reactive and sulfhydryl-reactive cross-linkers such as 4-(4-N-maleimidophenyl) butyric acid hydrazide (MPBH). 4-(N-maleimidomethyl) cyclohexane-1-carboxyl-hydrazide-8 (M2C2H). 3-(2-pyridyldithio) propionyl hydrazide (PDPH), amine-reactive and photoreactive cross-linkers such as N-hydroxysuccinimidyl-4-azidosalicylic acid (NHs-AsA). N-hydroxysulfosuccinimidyl-4-azidosalicylic acid (sulfo-NHs-AsA), sulfosuccinimidy 1-(4-azidosalicylamido)hexanoate(sulfo-NHs-LC-AsA), sulfosuccinimidyl-2-(p-azidosalicylamido) ethyl-1,3′-dithiopropionate (sAsD). N-hydroxysuccinimidyl-4-azidobenzoate (HsAB). N-hydroxysulfosuccinimidyl-4-azidobenzoate (sulfo-HsAB). N-succinimidyl-6-(4′-azido-2′-nitrophenylamino)hexanoate(SANPAH), sulfosuccinimidyl-6-(4′-azido-2′-nitrophenylamino)hexanoate(sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND). N-succinimidyl-4 (4-azidophenyl) 1,3′-dithiopropionate (sADP). N-sulfosuccinimidyl (4-azidophenyl)-1,3′-dithiopropionate (sulfo-sADP), sulfosuccinimidyl 4-(p-azidophenyl) butyrate (sulfo-sAPB), sulfosuccinimidyl 2-(7-azido-4-methylcoumarin-3-acetamide) ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3.3,3-trifluoropropionate (PNP-DTP), sulfhydryl-reactive and photoreactive cross-linkers such as 1-(p-Azidosalicylamido)-4-(iodoacetamido) butane (AsIB). N-[4-(p-azidosalicylamido) butyl]-3′-(2′-pyridyldithio) propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido) butylamine (AsBA), and arginine-reactive and photoreactive cross-linkers such as p-azidophenyl glyoxal (APG).

In some instances, the linker comprises a reactive functional group. In some cases, the reactive functional group comprises a nucleophilic group that is reactive to an electrophilic group present on an antibody. Exemplary electrophilic groups include carbonyl groups-such as aldehyde, ketone, carboxylic acid, ester, amide, enone, acyl halide or acid anhydride. In some aspects, the reactive functional group is aldehyde. Exemplary nucleophilic groups include hydrazide, oxime, amino, hydrazine, thiosemicarbazone, hydrazine carboxylate, and arylhydrazide.

In some aspects, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproxl (mc). In some cases, the linker is maleimidocaproyl (mc), such as 6-maleimidocaproic acid (MC). In other instances, the maleimide group comprises a maleimidomethyl group, such as succinimidyl-4-(N-maleimidomethyl) cyclohexane-1-carboxylate (sMCC) or sulfosuccinimidyl-4-(N-maleimidomethyl) cyclohexane-1-carboxylate (sulfo-sMCC) described above.

In some aspects, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al . . . “Self-hydrolyzing maleimides improve the stability and pharmacological properties of antibody-drug conjugates.” Nat. Biotechnol. 32 (10); 1059-1062 (2014). In some instances, the linker comprises a self-stabilizing maleimide. In some instances, the linker is a self-stabilizing maleimide.

In some aspects, the linker comprises a peptide moiety. In some instances, the peptide moiety comprises at least 2, 3, 4, 5, or 6 more amino acid residues. In some instances, the peptide moiety comprises at most 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline). Gly-Gly-Phe-Gly (SEQ ID NO; 70), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO; 71), or Gly-Phe-Leu-Gly (SEQ ID NO; 72). In some instances, the linker comprises a peptide moiety such as: Val-Cit (valine-citrulline), Gly-Gly-Phe-Gly (SEQ ID NO:70). Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO; 71), or Gly-Phe-Leu-Gly (SEQ ID NO:72). In some cases, the linker comprises Val-Cit. In some cases, the linker is Val-Cit.

In some aspects, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some aspects, the linker comprises one or more of a maleimide group, a peptide moiety, and/or a benzoic acid group, in any combination. In some aspects, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some aspects, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some aspects, the linker is a dendritic type linker. In some instances, the dendritic type linker comprises a branching, multifunctional linker moiety. In some instances, the dendritic type linker is used to increase the molar ratio of polynucleotide to the antibody. In some instances, the dendritic type linker comprises PAMAM dendrimers.

In some aspects, the linker is a traceless linker or a linker in which after cleavage does not leave behind a linker moiety (e.g., an atom or a linker group) to a binding moiety (e.g., an antibody), a polynucleotide, a polymer, or an endosomolytic moiety. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linkers. In some cases, the linker is a traceless aryl-triazene linker as described in Hejesen, et al . . . “A traceless aryl-triazene linker for DNA-directed chemistry.” Org Biomol Chem 11 (15); 2493-2497 (2013). In some instances, the linker is a traceless linker described in Blaney, et al . . . “Traceless solid-phase organic synthesis.” Chem. Rev. 102:2607-2024 (2002). In some instances, a linker is a traceless linker as described in U.S. Pat. No. 6,821,783.

In some instances, the linker is a linker described in U.S. Pat. Nos. 6,884,869; 7.498.298:8.288.352; 8.609.105: or 8.697.688; U.S. Patent Publication Nos. 2014/0127239: 2013/028919: 2014/286970: 2013/0309256: 2015/037360; or 2014/0294851: or PCT Publication Nos. WO2015057699; WO2014080251: WO2014197854: WO2014145090; or WO2014177042.

In some instances, the linker is a C1-C6 alkyl group. In some cases, the linker is a C1-C6 alkyl group, such as for example, a C5, C4, C3, C2, or CI alkyl group. In some cases, the C1-C6 alkyl group is an unsubstituted C₁-C₆ alkyl group. As used in the context of a linker, and in particular in the context of the linker, alkyl means a saturated straight or branched hydrocarbon radical containing up to six carbon atoms. In some instances, the linker is a non-polymeric linker. In some instances, the linker includes a homobifunctional linker or a heterobifunctional linker described supra. In some cases, the linker includes a heterobifunctional linker. In some cases, the linker includes or comprises sMCC. In other instances, the linker includes a heterobifunctional linker optionally conjugated to a C1-C6 alkyl group. In other instances, the linker includes sMCC optionally conjugated to a C1-C6 alkyl group. In additional instances, the linker does not include a homobifunctional linker or a heterobifunctional linker described supra. In some cases, the linker includes or comprises 6-maleimidocaproic acid (MC).

### Pharmaceutical Formulation

In some aspects, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition described herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition described herein is formulated for oral administration. In still other instances, the pharmaceutical composition described herein is formulated for intranasal administration.

In some aspects, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety (e.g., antibody described herein)). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, a-chymotrypsin, polylysine, polyarginine, histone, protamine, ovalbumin, or dextrin or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety (e.g., antibody) described herein is conjugated either directly or indirectly to the nanoparticle. In some instances, at least 1, 5, 10, 15, 20, 30, 40, 50, 60, 70, 80, 90, 100 or more polynucleic acid molecules or binding moieties described herein are conjugated either directly or indirectly to a nanoparticle.

In some aspects, the pharmaceutical formulation comprises a delivery vector, e.g., a recombinant vector, the delivery of the polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide for transient expression of polynucleic acid molecules.

In some aspects, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995): Hoover, John E., Remington's Pharmaceutical Sciences, Mack Publishing Co., Easton, Pennsylvania 1975: Liberman, H. A, and Lachman, L., Eds., Pharmaceutical Dosage Forms, Marcel Decker, New York, N.Y., 1980); and Pharmaceutical Dosage Forms and Drug Delivery Systems, Seventh Ed. (Lippincott Williams & Wilkins 1999).

### Therapeutic Regimens

In some aspects, the pharmaceutical compositions described herein are administered for therapeutic applications. In some aspects, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some aspects, one or more pharmaceutical compositions are administered simultaneously, sequentially, or at an interval period of time. In some aspects, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some aspects, two or more different pharmaceutical compositions are coadministered. In some instances, the two or more different pharmaceutical compositions are coadministered simultaneously. In some cases, the two or more different pharmaceutical compositions are coadministered sequentially without a gap of time between administrations. In other cases, the two or more different pharmaceutical compositions are coadministered sequentially with a gap of about 0.5 hours, 1 hour, 2 hours, 3 hours, 12 hours, 1 day, 2 days, or more between administrations.

In the case wherein the patient's status does improve, upon the doctor's discretion the administration of the composition is given continuously: alternatively, the dose of the composition being administered is temporarily reduced or temporarily suspended for a certain length of time (i.e., a “drug holiday”). In some instances, the length of the drug holiday varies between 2 days and I year, including by way of example only. 2 days, 3 days. 4 days, 5 days, 6 days. 7 days, 10 days, 12 days, 15 days, 20 days, 28 days, 35 days, 50 days, 70 days. 100 days. 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some aspects, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some aspects, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50) (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in humans. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain aspects, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include target nucleic acid molecule described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself: a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain aspects, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a.” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some aspects, the mammal is a human. In some aspects, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

As used here, the term “DMD patient” means any human that suffers or expected to suffer from DMD, and/or has a genetic predisposition (e.g., mutations in DMD gene) related to the DMD.

As used here, the term “a subject affected by DMD” means any mammal that suffers or expected to suffer from DMD, and/or has a genetic predisposition (e.g., mutations in DMD gene) related to the DMD. In some aspects, the mammal is a human. In some aspects, the mammal is a non-human.

### EXAMPLES

These examples are provided for illustrative purposes only and not to limit the scope of the claims provided herein.

### Example 1: Screening of 28-Mer Phosphorodiamidate Morpholino Oligomers (Pmos) Skipping Exon 50 of Dmd Gene in Healthy Human Skeletal Muscle Cells

### Pmo Library Design and Synthesis

Phosphorodiamidate morpholino oligomers (PMOs) were selected for their stability, solubility in aqueous solutions and low toxicity. For analytical purposes PMOs were custom-made by Gene Tools, LLC (Corvallis, USA). Exon 50 has a length of 109 nucleotides (source: NCBI Homo sapiens dystrophin (DMD) transcript variant Dp427m, mRNA. ACCESSION NM_004006.3); 5′-AGGAAGTTAGAAGATCTGAGCTCTGAGTGGAAGGCGGTAAACCGTTTACTTCAAGA GCTGAGGGCAAAGCAGCCTGACCTAGCTCCTGGACTGACCACTATTGGAGCCT-3′ (SEQ ID NO; 200).

The nucleic acid sequence of exon 50 was used as the template to design a PMO library of 44 sequences targeting DMD exon 50. The PMO target sequences have a length of 28 nucleotides (28-mer) and are tiled 2 nucleotides apart across the exon, resulting in a library of 44 PMO sequences as shown in Table 1. PMO nomenclature was based on targeted exon, and distance from the end of the acceptor site (start of the exon) (e.g., Ex50-1).

TABLE 1

| | | PMO | | SEQ |
|---|---|---|---|---|
| PMO | Target | Length | | ID |
| Name | Exon | (bp) | PMO Sequence (5′-3′) | NO: |
| Ex50-1 | 50 | 28 | GAGCTCAGATCTTCTAACTTCCTCTTCA | 100 |
| Ex50-2 | 50 | 28 | CAGAGCTCAGATCTTCTAACTTCCTCTT | 101 |
| Ex50-3 | 50 | 28 | CTCAGAGCTCAGATCTTCTAACTTCCTC | 102 |
| Ex50-4 | 50 | 28 | CACTCAGAGCTCAGATCTTCTAACTTCC | 103 |
| Ex50-5 | 50 | 28 | TCCACTCAGAGCTCAGATCTTCTAACTT | 104 |
| Ex50-6 | 50 | 28 | CTTCCACTCAGAGCTCAGATCTTCTAAC | 105 |
| Ex50-7 | 50 | 28 | GCCTTCCACTCAGAGCTCAGATCTTCTA | 106 |
| Ex50-8 | 50 | 28 | CCGCCTTCCACTCAGAGCTCAGATCTTC | 107 |
| Ex50-9 | 50 | 28 | TACCGCCTTCCACTCAGAGCTCAGATCT | 108 |
| Ex50-10 | 50 | 28 | TTTACCGCCTTCCACTCAGAGCTCAGAT | 109 |
| Ex50-11 | 50 | 28 | GGTTTACCGCCTTCCACTCAGAGCTCAG | 110 |
| Ex50-12 | 50 | 28 | ACGGTTTACCGCCTTCCACTCAGAGCTC | 111 |
| Ex50-13 | 50 | 28 | AAACGGTTTACCGCCTTCCACTCAGAGC | 112 |
| Ex50-14 | 50 | 28 | GTAAACGGTTTACCGCCTTCCACTCAGA | 113 |
| Ex50-15 | 50 | 28 | AAGTAAACGGTTTACCGCCTTCCACTCA | 114 |
| Ex50-16 | 50 | 28 | TGAAGTAAACGGTTTACCGCCTTCCACT | 115 |
| Ex50-17 | 50 | 28 | CTTGAAGTAAACGGTTTACCGCCTTCCA | 116 |
| Ex50-18 | 50 | 28 | CTCTTGAAGTAAACGGTTTACCGCCTTC | 117 |
| Ex50-19 | 50 | 28 | AGCTCTTGAAGTAAACGGTTTACCGCCT | 118 |
| Ex50-20 | 50 | 28 | TCAGCTCTTGAAGTAAACGGTTTACCGC | 119 |
| Ex50-21 | 50 | 28 | CCTCAGCTCTTGAAGTAAACGGTTTACC | 120 |
| Ex50-22 | 50 | 28 | GCCCTCAGCTCTTGAAGTAAACGGTTTA | 121 |
| Ex50-23 | 50 | 28 | TTGCCCTCAGCTCTTGAAGTAAACGGTT | 122 |
| Ex50-24 | 50 | 28 | CTTTGCCCTCAGCTCTTGAAGTAAACGG | 123 |
| Ex50-25 | 50 | 28 | TGCTTTGCCCTCAGCTCTTGAAGTAAAC | 124 |
| Ex50-26 | 50 | 28 | GCTGCTTTGCCCTCAGCTCTTGAAGTAA | 125 |
| Ex50-27 | 50 | 28 | AGGCTGCTTTGCCCTCAGCTCTTGAAGT | 126 |
| Ex50-28 | 50 | 28 | TCAGGCTGCTTTGCCCTCAGCTCTTGAA | 127 |
| Ex50-29 | 50 | 28 | GGTCAGGCTGCTTTGCCCTCAGCTCTTG | 128 |
| Ex50-30 | 50 | 28 | TAGGTCAGGCTGCTTTGCCCTCAGCTCT | 129 |
| Ex50-31 | 50 | 28 | GCTAGGTCAGGCTGCTTTGCCCTCAGCT | 130 |
| Ex50-32 | 50 | 28 | GAGCTAGGTCAGGCTGCTTTGCCCTCAG | 131 |
| Ex50-33 | 50 | 28 | AGGAGCTAGGTCAGGCTGCTTTGCCCTC | 132 |
| Ex50-34 | 50 | 28 | CCAGGAGCTAGGTCAGGCTGCTTTGCCC | 133 |
| Ex50-35 | 50 | 28 | GTCCAGGAGCTAGGTCAGGCTGCTTTGC | 134 |
| Ex50-36 | 50 | 28 | CAGTCCAGGAGCTAGGTCAGGCTGCTTT | 135 |
| Ex50-37 | 50 | 28 | GTCAGTCCAGGAGCTAGGTCAGGCTGCT | 136 |
| Ex50-38 | 50 | 28 | TGGTCAGTCCAGGAGCTAGGTCAGGCTG | 137 |
| Ex50-39 | 50 | 28 | AGTGGTCAGTCCAGGAGCTAGGTCAGGC | 138 |
| Ex50-40 | 50 | 28 | ATAGTGGTCAGTCCAGGAGCTAGGTCAG | 139 |
| Ex50-41 | 50 | 28 | CAATAGTGGTCAGTCCAGGAGCTAGGTC | 140 |
| Ex50-42 | 50 | 28 | TCCAATAGTGGTCAGTCCAGGAGCTAGG | 141 |
| Ex50-43 | 50 | 28 | GCTCCAATAGTGGTCAGTCCAGGAGCTA | 142 |
| Ex50-44 | 50 | 28 | AGGCTCCAATAGTGGTCAGTCCAGGAGC | 143 |

### Cell Culture Conditions

Healthy immortalized skeletal muscle cells (Cell ID: AB1167) were obtained from the Association Institut de Myologie—Centre de Recherche en Myologie, UMRS 787 INSERM and Sorbonne Université, France. These cells were isolated from healthy donors and immortalized as previously described (K. Mamchaoui et al., “Immortalized pathological human myoblasts: Towards a universal tool for the study of neuromuscular disorders,” Skelet Muscle, vol. 1, no. 1, November 2011, doi; 10.1186/2044-5040-1-34). Healthy immortalized skeletal muscle cells were grown in skeletal muscle growth media (Promocell, Cat. No. C-23160). Media supplements and their respective final concentrations were as follows: fetal calf serum (0.05 ml/ml), fetuin (bovine) (50 μg/ml), epidermal growth factor (recombinant human) (10 ng/ml), basic fibroblast growth factor (recombinant human) (1 ng/ml), insulin (recombinant human) (10 μg/ml), dexamethasone (0.4 μg/ml) and 0.5% gentamycin (Gibco, Cat. No. 15750078). Healthy immortalized skeletal muscle cells were differentiated in skeletal muscle differentiation media which contained DMEM with Glutamax supplemented with Skeletal Muscle Cell Differentiation Medium Supplement Mix (Promocell, Cat. No. C-39366) and 0.5% gentamycin (Gibco, Cat. No. 15750078).

Cells were seeded in 96-well (Costar, Cat. No. 3596) or 24-well (Costar, Cat. No. 3524) tissue culture plates coated with 1% Matrigel (Corning, Cat. No. 356234) in growth media (GM) on day 0. At approximately 80%-90% confluence, myogenic differentiation was induced by replacing GM with differentiation media (DM) after 72 hours (day 3). PMOs were heated at 65-70° C., for 5-10 minutes, diluted into warm medium and added to cells to allow gymnotic uptake 48 hours after switching to DM (day 5). Cells were collected in Trizol 48 hours after PMO treatments (day 7) and stored at −80° C. until further processing for RNA isolation using Direct-zol-96 RNA isolation kit (Zymo, Cat. No. R2056) according to the manufacturer's instructions. 100-500 ng of purified RNA was converted to cDNA using High-Capacity cDNA Reverse Transcription Kit (Applied Biosystems, Cat. No. 4368813) and a SimpliAmp Thermal Cycler (Applied Biosystems, Cat. No. A24811).

### Exon Skipping Analysis (RT-qPCR)

TaqMan™ Fast Advanced Master Mix (Applied Biosystems, Cat. No. 4444558) and TaqMan probes were added to cDNA and loaded in 384 well plates in duplicates. RT-qPCR was conducted using QuantStudio 6 or 7 Flex Real-Time PCR instruments from Applied Biosystems. Data were analyzed by QuantStudio™ Real-Time PCR Software (v1.7.2, Applied Biosystems). AACt method was used to determine the expression of Exon 50-skipped DMD mRNA.

DNA fragments representing total DMD mRNA or exon 50-skipped DMD mRNAs were amplified by RT-qPCR using TaqMan Fast Advanced Master mix (Applied Biosystems) and either a hDMD TaqMan assay Hs01049401_ml (VIC-MGB, Thermo Fisher Scientific) or a custom-made TaqMan assay specific for the hDMD exon 49/51 junction (FAM-MGB, Forward: 5′-TCTAAAGGGCAGCATTTGT-3′ (SEQ ID NO:80), Reverse; 5′-GGAGATGGCAGTTTCCTTAG-3′ (SEQ ID NO:81), Probe; 5′-TCAGCCAGTGAAGCTCCTACTCAGA-3′ (SEQ ID NO:82)), Data were normalized to AHSAI (reference gene) presented as mean of skipped DMD expression relative to mock (mean (triplicates)±SEM). Statistical significance was assessed using ANOVA with Bonferroni's Multiple Comparisons test (*p<0).05). Statistical significance was defined as p<0.05 and represented by an asterisk (*).

### Results

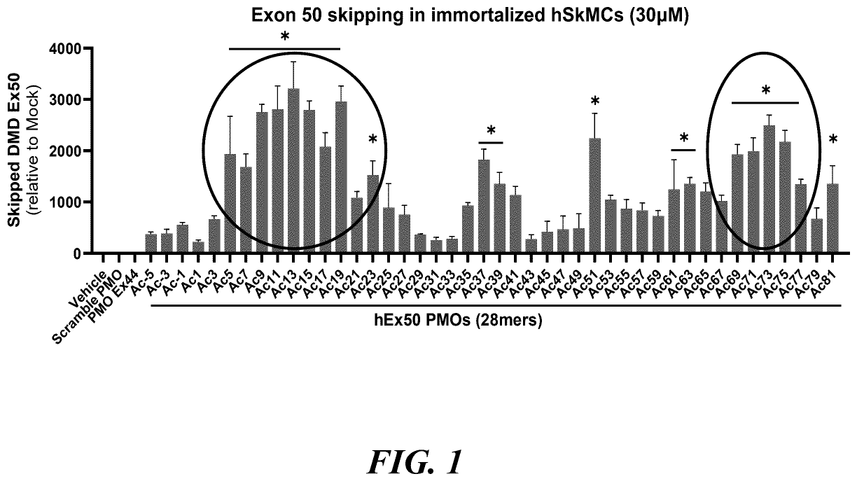

Screening of the 44 PMOs for skipping exon 50 was performed in healthy human immortalized myotubes. The assay revealed that numerous PMOs from the library have higher exon 50 skipping activities than the activities of the other PMOs (FIG. 1). Among the PMOs with high exon 50 skipping activity. 2 different clusters in region of interest (ROI) of exon 50 were found (see circles). The first ROI is located between 5 nucleotides (Ac5) and 19 nucleotides (Ac19) from the end of the acceptor site of exon 50 and the second ROI is located between 61 nucleotides (Ac61) and 77 nucleotides from acceptor site of exon 50.

Overall, PMOs with significant exon 50 skipping activities cluster at 2 different ROIs of exon 50.

### Example 2: Identification and Selection of PMO with High Exon 50 Skipping Activity in Healthy Primary Human Skeletal Muscle Cells (hSkMCs)

The design and synthesis of the PMOs are described in Example 1. The nomenclature of the selected 11 PMOs is based on targeted exon, distance from the end of the acceptor site (start of the exon), and the respective PMO length (e.g., hEx50_Ac9_28mer). PMO concentrations were measured in house by nanodrop. The PMOs are described in Table 2.

TABLE 2

| | | PMO | | SEQ |
|---|---|---|---|---|
| | Target | Length | | ID |
| PMO Name | Exon | (bp) | PMO Sequence (5′-3′) | NO: |
| hEx50_Ac9_28 mer | 50 | 28 | CCGCCTTCCACTCAGAGCTCAGATCTTC | 107 |
| hEx50_Ac11_28 mer | 50 | 28 | TACCGCCTTCCACTCAGAGCTCAGATCT | 108 |
| hEx50_Ac13_28 mer | 50 | 28 | TTTACCGCCTTCCACTCAGAGCTCAGAT | 109 |
| hEx50_Ac15_28 mer | 50 | 28 | GGTTTACCGCCTTCCACTCAGAGCTCAG | 110 |
| hEx50_Ac17_28 mer | 50 | 28 | ACGGTTTACCGCCTTCCACTCAGAGCTC | 111 |
| hEx50_Ac29_28 mer | 50 | 28 | CTCTTGAAGTAAACGGTTTACCGCCTTC | 117 |
| hEx50_Ac37_28 mer | 50 | 28 | GCCCTCAGCTCTTGAAGTAAACGGTTTA | 121 |
| hEx50_Ac69_28 mer | 50 | 28 | TGGTCAGTCCAGGAGCTAGGTCAGGCTG | 137 |
| hEx50_Ac71_28 mer | 50 | 28 | AGTGGTCAGTCCAGGAGCTAGGTCAGGC | 138 |
| hEx50_Ac73_28 mer | 50 | 28 | ATAGTGGTCAGTCCAGGAGCTAGGTCAG | 139 |
| hEx50_Ac79_28 mer | 50 | 28 | GCTCCAATAGTGGTCAGTCCAGGAGCTA | 142 |

### Cell Culture Conditions

Healthy immortalized skeletal muscle cells (AB1167) were obtained from the Association Institut de Myologie-Centre de Recherche en Myologie, UMRS 787 INSERM and Sorbonne Université, France. These cells were isolated from healthy donors and immortalized as previously described (K. Mamchaoui et al., “Immortalized pathological human myoblasts: Towards a universal tool for the study of neuromuscular disorders,” Skelet Muscle, vol. 1, no. 1, November 2011, doi; 10.1186/2044-5040-1-34). Healthy immortalized skeletal muscle cells were grown in skeletal muscle growth media (Promocell, Cat. No. C-23160). Media supplements and their respective final concentrations were as follows: fetal calf serum (0.05 ml/ml), fetuin (bovine) (50 μg/ml), epidermal growth factor (recombinant human) (10 ng/ml), basic fibroblast growth factor (recombinant human) (1 ng/ml), insulin (recombinant human) (10 μg/ml), dexamethasone (0.4 μg/ml) and 0.5% gentamycin (Gibco, Cat. No. 15750078). Healthy immortalized skeletal muscle cells were differentiated in skeletal muscle differentiation media which contained DMEM with Glutamax supplemented with Skeletal Muscle Cell Differentiation Medium Supplement Mix (Promocell, Cat. No. C-39366) and 0.5% gentamycin (Gibco, Cat. No. 15750078).

### Exon Skipping Analysis (RT-qPCR)

TaqMan™ Fast Advanced Master Mix (Applied Biosystems. Cat. No. 4444558) and TaqMan probes were added to cDNA and loaded in 384 well plates in duplicates. RT-qPCR was conducted using QuantStudio 6 or 7 Flex Real-Time PCR instruments from Applied Biosystems. Data were analyzed by QuantStudio™ Real-Time PCR Software (v1.7.2. Applied Biosystems). AACt method was used to determine the expression of Exon 50-skipped DMD mRNA.

DNA fragments representing total DMD mRNA or exon 50-skipped DMD mRNAs were amplified by RT-qPCR using TaqMan Fast Advanced Master mix (Applied Biosystems) and either a hDMD TaqMan assay Hs01049401_ml (VIC-MGB. Thermo Fisher Scientific), a custom-made TaqMan assay specific for the hDMD exon 49/51 junction (FAM-MGB. Forward:

(SEQ ID NO: 80)
5′-TCTAAAGGGCAGCATTTGT-3′,
Reverse:
(SEQ ID NO: 81)
5′-GGAGATGGCAGTTTCCTTAG-3′,
Probe:
(SEQ ID NO:82))
5′-TCAGCCAGTGAAGCTCCTACTCAGA-3′.

Data were normalized to AHSAI (reference gene) and are presented as mean of skipped DMD expression relative to mock (mean (triplicates)±SEM). Statistical significance was assessed using ANOVA with Dunnett's Multiple Comparisons test (*p<0.05). Statistical significance was defined as p<0).05 and represented by an asterisk (*).

### Results

Selected 10 PMOs having high exon 50 skipping activity and 2 PMOs having low exon 50 skipping activity from Example 1 were further evaluated in a dose dependent response in in vitro assays in healthy primary human skeletal muscle cells (hSkMCs). The selected PMOs were transfected at concentrations of 0.3 μM. I μM. 3 μM, 10 μM, and 30 μM in healthy primary hSkMCs that were pre-differentiated into myotubes as described above and harvested 48 hours post-transfection. Total DMD mRNAs and exon 50 skipped DMD mRNAs were amplified by RT-qPCR. PCR products were separated by gel electrophoresis and quantified by densitometry.

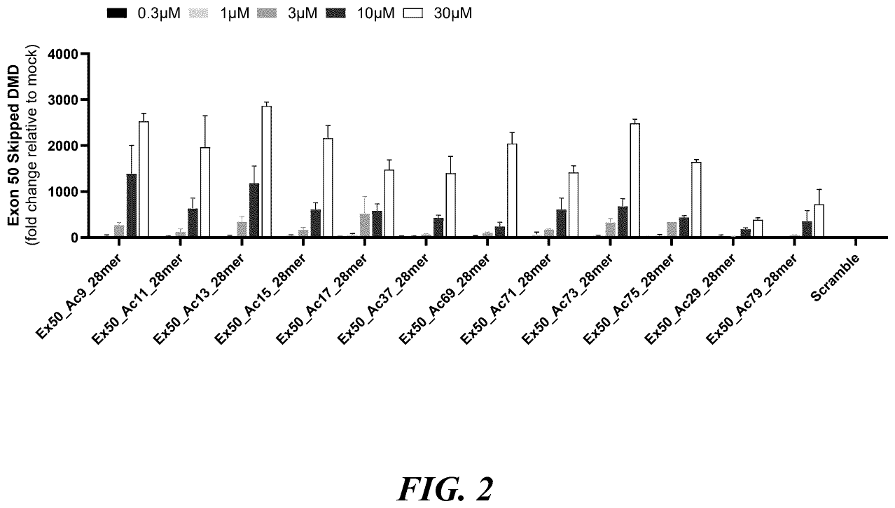

FIG. 2 shows the dose dependent response of the exon 50 skipping activity of the selected 10 PMOs having high exon 50 skipping activity and 2 PMOs having low exon 50 skipping activity (28-mers) targeting the human DMD exon 50 in healthy primary hSkMCs. Results of the assay in hSkMCs indicated that the 9 PMOs in the 2 regions of interest (ROI) and 1 PMO falling in between the two ROIs, were able to induce exon 50 skipping activities in a dose dependent manner (FIG. 2), while the 2 PMOs outside the ROI had lower exon 50 skipping activity. More specifically, the PMOs Ex50_Ac29_28 and Ex_Ac79_28 had lower levels of exon 50 skipping activities, while the highest levels of exon 50 skipping activity were measured at a concentration of 30 μM with the PMO Ex50_Ac13_28, which had a 3000-fold increase in exon 50 skipping activity compared to mock treated cells.

Overall, the PMOs identified in the 2 regions of interest could effectively induce exon 50 skipping in a concentration dependent manner in healthy primary human skeletal muscle cells.

### Example 3: Selection of Length-Optimized Exon 50 Skipping PMO in Healthy Immortalized Human Skeletal Muscle Cells (hSkMCs)

Three different libraries with PMO sequences that have a length of 22 nucleotides (22-mer), 24 nucleotides (24-mer), 26 nucleotides (26-mer) targeting in the 2 previously identified ROIs in exon 50 in Example 1, were designed to identify PMOs with a length of less than 30 nucleotides. The PMO sequences that have a length of 22 nucleotides (22-mer), 24 nucleotides (24-mer), 26 nucleotides (26-mer), or 28 nucleotides (28-mer) are shown in Table 3. These shortened PMOs were designed to target the sequences in the ROI identified in Example 1. The PMO sequences with the same length are spaced apart by 2 nucleotides (e.g., hEx50_Ac15_22mer is 2 nucleotides apart from the hEx50_Ac17_22mer). The synthesis of the PMOs is described in Example 1 and the nomenclature of the PMOs is based on targeted exon, distance from the acceptor site (start of the exon), and the respective PMO length (e.g., hEx50_Acl_28mer).

TABLE 3

| | | PMO | | SEQ |
|---|---|---|---|---|
| | Target | Length | | ID |
| PMO Name | Exon | (bp) | PMO Sequence (5′-3′) | NO: |
| hEx50_Ac15_22 mer | 50 | 22 | CCGCCTTCCACTCAGAGCTCAG | 144 |
| hEx50_Ac17_22 mer | 50 | 22 | TACCGCCTTCCACTCAGAGCTC | 145 |
| hEx50_Ac73_22 mer | 50 | 22 | GTCAGTCCAGGAGCTAGGTCAG | 146 |
| hEx50_Ac75_22 mer | 50 | 22 | TGGTCAGTCCAGGAGCTAGGTC | 147 |
| hEx50_Ac9_24 mer | 50 | 24 | CTTCCACTCAGAGCTCAGATCTTC | 148 |
| hEx50_Ac11_24 mer | 50 | 24 | GCCTTCCACTCAGAGCTCAGATCT | 149 |
| hEx50_Ac13_24 mer | 50 | 24 | CCGCCTTCCACTCAGAGCTCAGAT | 150 |
| hEx50_Ac15_24 mer | 50 | 24 | TACCGCCTTCCACTCAGAGCTCAG | 151 |
| hEx50_Ac17_24 mer | 50 | 24 | TTTACCGCCTTCCACTCAGAGCTC | 152 |
| hEx50_Ac19_24 mer | 50 | 24 | GGTTTACCGCCTTCCACTCAGAGC | 153 |
| hEx50_Ac21_24 mer | 50 | 24 | ACGGTTTACCGCCTTCCACTCAGA | 154 |
| hEx50_Ac69_24 mer | 50 | 24 | CAGTCCAGGAGCTAGGTCAGGCTG | 155 |
| hEx50_Ac71_24 mer | 50 | 24 | GTCAGTCCAGGAGCTAGGTCAGGC | 156 |
| hEx50_Ac73_24 mer | 50 | 24 | TGGTCAGTCCAGGAGCTAGGTCAG | 157 |
| hEx50_Ac75_24 mer | 50 | 24 | AGTGGTCAGTCCAGGAGCTAGGTC | 158 |
| hEx50_Ac77_24 mer | 50 | 24 | ATAGTGGTCAGTCCAGGAGCTAGG | 159 |
| hEx50_Ac79_24 mer | 50 | 24 | CAATAGTGGTCAGTCCAGGAGCTA | 160 |
| hEx50_Ac9_26 mer | 50 | 26 | GCCTTCCACTCAGAGCTCAGATCTTC | 161 |
| hEx50_Ac11_26 mer | 50 | 26 | CCGCCTTCCACTCAGAGCTCAGATCT | 162 |
| hEx50_Ac13_26 mer | 50 | 26 | TACCGCCTTCCACTCAGAGCTCAGAT | 163 |
| hEx50_Ac15_26 mer | 50 | 26 | TTTACCGCCTTCCACTCAGAGCTCAG | 164 |
| hEx50_Ac17_26 mer | 50 | 26 | GGTTTACCGCCTTCCACTCAGAGCTC | 165 |
| hEx50_Ac69_26 mer | 50 | 26 | GTCAGTCCAGGAGCTAGGTCAGGCTG | 166 |
| hEx50_Ac71_26 mer | 50 | 26 | TGGTCAGTCCAGGAGCTAGGTCAGGC | 167 |
| hEx50_Ac73_26 mer | 50 | 26 | AGTGGTCAGTCCAGGAGCTAGGTCAG | 168 |
| hEx50_Ac75_26 mer | 50 | 26 | ATAGTGGTCAGTCCAGGAGCTAGGTC | 169 |
| hEx50_Ac9_28 mer | 50 | 28 | CCGCCTTCCACTCAGAGCTCAGATCTTC | 107 |
| hEx50_Ac13_28 mer | 50 | 28 | TTTACCGCCTTCCACTCAGAGCTCAGAT | 109 |
| hEx50_Ac73_28 mer | 50 | 28 | ATAGTGGTCAGTCCAGGAGCTAGGTCAG | 139 |

### Cell Culture Conditions

Healthy immortalized skeletal muscle cells (AB1167) were obtained from the Association Institut de Myologie-Centre de Recherche en Myologie, UMRS 787 INSERM and Sorbonne Université, France. These cells were isolated from healthy donors and immortalized as previously described (K. Mamchaoui et al., “Immortalized pathological human myoblasts: Towards a universal tool for the study of neuromuscular disorders,” Skelet Muscle, vol. 1, no. 1, November 2011, doi; 10.1186/2044-5040-1-34). Healthy immortalized skeletal muscle cells were grown in skeletal muscle growth media (Promocell, Cat. No. C-23160). Media supplements and their respective final concentrations were as follows: fetal calf serum (0.05 ml/ml), fetuin (bovine) (50 μg/ml), epidermal growth factor (recombinant human) (10 ng/ml), basic fibroblast growth factor (recombinant human) (1 ng/ml), insulin (recombinant human) (10 μg/ml), dexamethasone (0.4 μg/ml) and 0.5% gentamycin (Gibco, Cat. No. 15750078). Healthy immortalized skeletal muscle cells were differentiated in skeletal muscle differentiation media which contained DMEM with Glutamax supplemented with Skeletal Muscle Cell Differentiation Medium Supplement Mix (Promocell, Cat. No. C-39366) and 0.5% gentamycin (Gibco, Cat. No. 15750078).

### Exon Skipping Analysis (RT-qPCR)

TaqMan™ Fast Advanced Master Mix (Applied Biosystems, Cat. No. 4444558) and TaqMan probes were added to cDNA and loaded in 384 well plates in duplicates. RT-qPCR was conducted using QuantStudio 6 or 7 Flex Real-Time PCR instruments from Applied Biosystems. Data were analyzed by QuantStudio™ Real-Time PCR Software (v1.7.2, Applied Biosystems). AACt method was used to determine the expression of Exon 50-skipped DMD mRNA.

DNA fragments representing total DMD mRNA or exon 50-skipped DMD mRNAs were amplified by RT-qPCR using TaqMan Fast Advanced Master mix (Applied Biosystems) and either a hDMD TaqMan assay Hs01049401_ml (VIC-MGB, Thermo Fisher Scientific) or a custom-made TaqMan assay specific for the hDMD exon 49/51 junction (FAM-MGB, Forward: 5′-TCTAAAGGGCAGCATTTGT-3′ (SEQ ID NO:80), Reverse; 5′-GGAGATGGCAGTTTCCTTAG-3′ (SEQ ID NO:81), Probe; 5′-TCAGCCAGTGAAGCTCCTACTCAGA-3′) (SEQ ID NO:82), Data were normalized to AHSAI (reference gene) and presented as mean of skipped DMD expression relative to mock (mean (triplicates)±SEM). Statistical significance was assessed using ANOVA with Dunnett's Multiple Comparisons test (*p<0.05). Statistical significance was defined as p<0.05 and represented by an asterisk (*).

### Results

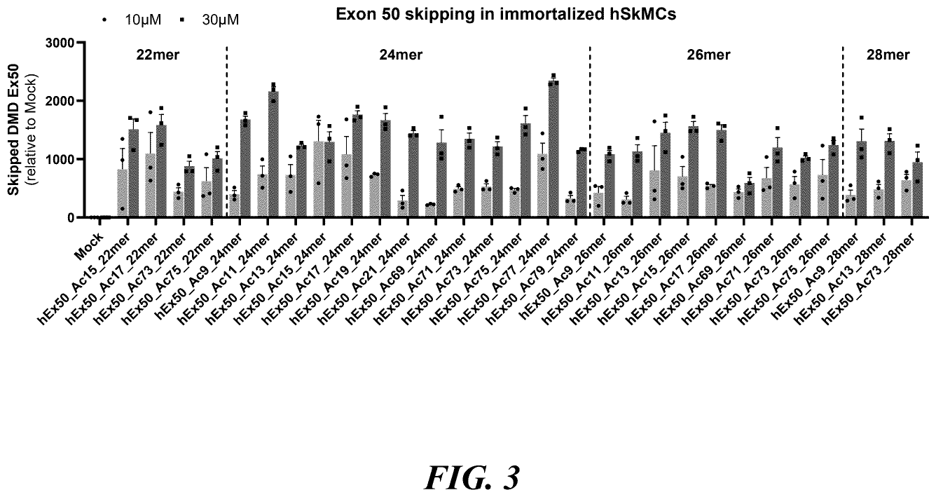

PMO libraries of 22-mer, 24-mer, and 26-mer were designed based on the exon 50 skipping data from the 2 ROIs of the 28-mer screen in Example 1. The 22-mer, 24-mer and 26-mer PMO oligonucleotides targeting sites on exon 50, which achieved maximum activity in Example 1, were screened and evaluated in vitro for exon 50 skipping activity, together with the top three 28-mer PMO50 sequences, in healthy hSkMC (AB1167) at two different concentrations of 10 μM and 30 μM (FIG. 3). In the focused PMO50 library, 22-mer, 24-mer, 26-mer of the Ac17, and 24-mer of the Ac77-targeting PMOs were among the most potent PMOs and demonstrated significant increase in exon 50 skipping activity relative to mock at all concentrations (FIG. 3). The focused library screens revealed multiple short PMOs with equal or higher levels of exon skipping compared to the 28mer PMOs. The library with the 24-mers had the best exon skipping activities. Based on the screening of the 3 libraries, the following 3 PMO sequences targeting at Ac15, Ac17, and Ac77 (15, 17, or 77 nucleotides from of the exon 50) acceptor site (start of the exon) were selected for further evaluation: hEx50_Ac15_24, hEx50_Ac17_24, and hEx50_Ac77_24. The 24-mer PMOs targeting the positions 15, 17, or 77 had the best exon 50 skipping activities.

### Example 4: Exon 50 Skipping Activity in DMD Patient-Derived Skeletal Muscle Cells with Deletion of Exon 51 (AEx51)

Primary skeletal muscle cells derived from DMD patients with a deletion of exon 51 in the DMI) gene (AEx51 DMD patient-derived skeletal muscle cells) were obtained from Carlo Besta Neurological Institute (Milan, Italy) (Cell ID; 44328). Primary cells were grown in GM composed of DMEM with Glutamax (Gibco, Cat. No. 10566-016), supplemented with 20% FBS (Nucleus Biologics, Cat. No. FBS1824-001), 10 μg/ml Insulin (Sigma-Aldrich, Cat. No. 10516-5 ml), 25 ng/ml hFGF (Stemcell Technologies, Cat. No. 78003), 10 ng/ml EGF (Stemcell Technologies, Cat. No. 78006.1), and 1% Penicillin and Streptomycin (Thermofisher, Cat. No. 15140122). Primary cells were differentiated in primary cell differentiation media which contained DMEM with Glutamax supplemented with Skeletal Muscle Cell Differentiation Medium Supplement Mix (Promocell. Cat. No. C-39366) and 1% Penicillin and Streptomycin (Thermofisher. Cat. No. 15140122).

Cells were seeded in 96-well (Costar. Cat. No. 3596) or 24-well (Costar. Cat. No. 3524) tissue culture plates coated with 1% Matrigel (Corning. Cat. No. 356234) in growth media (GM) on day 0). At approximately 80%-90% confluence, myogenic differentiation was induced by replacing GM with differentiation media (DM) after 72 hours (day 3). PMOs were heated at 65-70° C., for 5-10 minutes, diluted into warm medium and added to cells to allow gymnotic uptake 48 hours after switching to DM (day 5). Cells were collected in Trizol 48 hours after PMO treatments (day 7) and stored at −80° C. until further processing for RNA isolation using Direct-zol-96 RNA isolation kit (Zymo. Cat. No. R2056) according to the manufacturer's instructions. 100-500 ng of purified RNA was converted to cDNA using High-Capacity cDNA Reverse Transcription Kit (Applied Biosystems. Cat. No. 4368813) and a SimpliAmp Thermal Cycler (Applied Biosystems. Cat. No. A24811).

Exon Skipping Analysis (ddPCR)

cDNA was combined with Taqman probes. 2x ddPCR Supermix (no dUTP) (BioRad. Cat. No. 1863024), and BamHI restriction enzyme (New England BioLabs. Cat. No. R3136S) and loaded in 96 well plates for ddPCR (Bio-Rad. Cat. No. 12001925) in duplicates. Samples were partitioned into droplets in the Automated Droplet Generator (Bio-Rad. Cat. No. 1864100) and transferred to a deep well C1000 Touch Thermal Cyclers (Bio-Rad. Cat. No. 1851197) for PCR amplification of target genes. DNA fragments representing total DMD or Exon 50-skipped DMD mRNAs were amplified by PCR using TaqMan Fast Advanced Master mix (Applied Biosystems) and either a hDMD TaqMan assay Hs01049401_ml (VIC-MGB. Thermo Fisher Scientific) or a custom-made TaqMan assay specific for the hDMD exon 49-52 junction (FAM-MGB. Forward; 5′-GCAGTTCAAGCTAAACAACCG-3″ (SEQ ID NO; 86). Reverse; 5′-TTGTTGCCTTCACTGGCT-3′ (SEQ ID NO; 87), Probe; 5′-TGTCTAAAGGGCAGCATTTGTACAAGGA-3′ (SEQ ID NO; 88). Following target amplification, samples were read in QX200 Droplet Reader (Bio-Rad. Cat. No. 1864100) for absolute quantification of target mRNA transcripts. Data were analyzed using the QX Manager software (version 1.2. BioRad). Exon skipping data are presented as percent ratio of skipped DMD transcripts and total DMD transcripts. Statistical significance was assessed using ANOVA with Dunnett's Multiple Comparisons test (*p<0.05). Statistical significance was defined as p<0.05 and represented by an asterisk (*).

### Dystrophin Quantification by Jess Western Blotting System

Protein lysates were prepared and normalized based on total protein concentrations determined using the BCA method. Dystrophin restoration was quantified using the Simple Western Automated Western Blot System (ProteinSimple. Cat. No. 004-650) as per the manufacturer's instructions. The separation of proteins was achieved using a 66-440 kDa Separation Module (ProteinSimple. Cat. No. SM-W008). Dystrophin was detected using the Anti-Rabbit Detection Module (ProteinSimple. Cat. No. DM-001) with a rabbit monoclonal anti-dystrophin antibody (Abcam ab154168) diluted 1:1000 in Antibody Dilution Buffer (ProteinSimple. Cat. No. 042-203). Data were analyzed using Compass for SW software (6.2.0). The automatic peak detection feature was used to identify dystrophin peaks. Resulting electropherograms were reviewed and manual corrections were applied made if necessary. Dystrophin peaks were quantified by calculating the area under the curve (AUC). Dystrophin AUC was normalized to total protein AUC (total protein detection module). The software criteria for distinguishing low dystrophin signals from background included a peak signal-to-noise (S/N) ratio≥10 and a peak height/baseline ratio≥3, following a protocol adapted from Beekman et al . . . 2018. Data are normalized to total protein and presented as percent dystrophin expression relative to healthy myotubes (AB1167) dystrophin levels (mean (technical duplicates or triplicates)±SEM). Statistical significance was assessed using ANOVA with Dunnett's Multiple Comparisons test (*p<0.05).

### Results

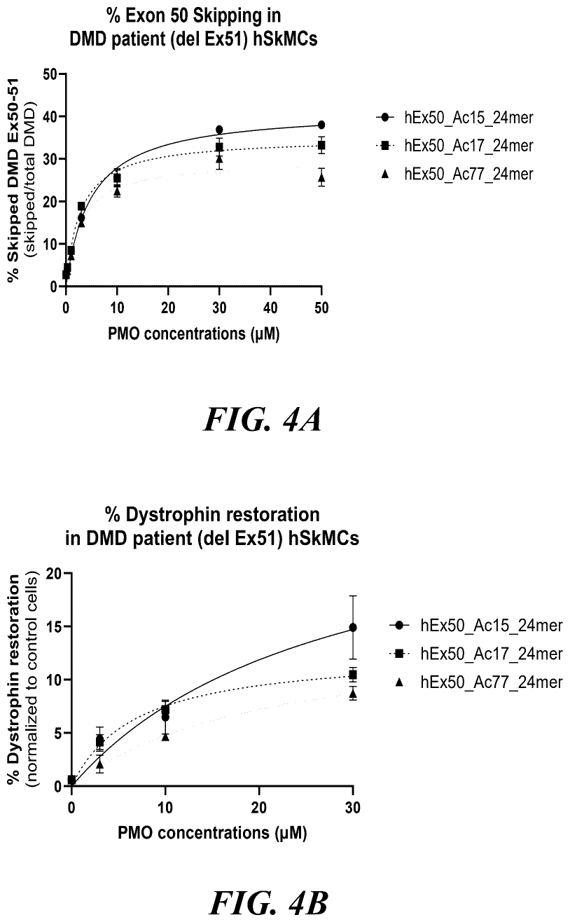

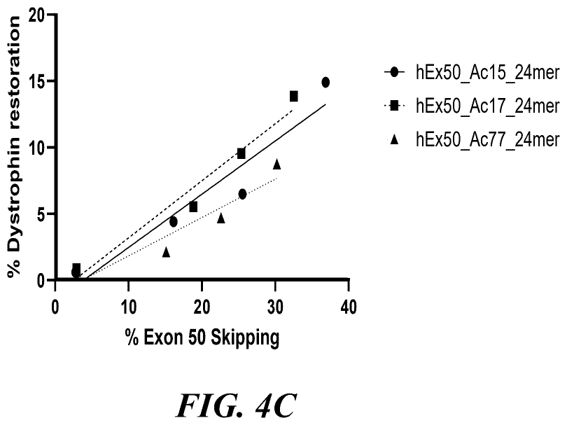

The 24-mer PMOs identified as being effective in Example 3 (hEx50_Ac15_24, hEx50_Ac17_24, and hEx50_Ac77_24) were further evaluated for exon 50 skipping activities at concentrations of 0.3 μM 1 μM. 10 μM and 30 μM via droplet digital PCR (ddPCR) in AEx51 DMD patient-derived skeletal muscle cells, which are amenable to exon 50 skipping. The 24-mer PMOs targeting exon 50 at a distance from the acceptor site of 15 (Ac15), 17 (Ac17), and 77 (Ac77) nucleotides showed dose-dependent exon 50 skipping activity in AEx51 DMD patient-derived skeletal muscle cells (FIG. 4A). The PMO with the highest exon 50 skipping activity is hEx50_Ac15_24 with about 30% the highest exon 50 skipping activity as determined by ddPCR at a concentration of 30 μM.

In addition, the 24-mer PMOs identified in Example 3 (hEx50_Ac15_24, hEx50)_Ac17_24, and hEx50_Ac77_24) were further evaluated for dystrophin protein restoration levels at concentrations of I μM. 10 UM and 30 μM via western blotting in AEx51 DMD patient-derived skeletal muscle cells (FIG. 4B). The PMO hEx50-Ac15_24 induced the highest level of dystrophin restoration as determined by western blotting with the Jess system.

A plot between the exon 50 skipping activity and dystrophin restoration levels revealed a high degree of correlation between exon 50 skipping activity using ddPCR and dystrophin restoration levels (FIG. 4C). The correlation coefficients of each PMO calculated from the plot (Table 11) confirming that increased exon 50 skipping activity correlate with increased dystrophin restoration levels.

TABLE 11

| PMO Name | Pearson r | R² |
|---|---|---|
| hEx_Ac15_24 | 0.9581 | 0.918 |
| hEx_Ac17_24 | 0.9799 | 0.9602 |
| hEx_Ac77_24 | 0.9463 | 0.8956 |

The PMO hEx_Ac17_24 has the best correlation coefficient confirming the exon 50 skipping activities correlates with increased dystrophin restoration levels.

Overall, the 24-mer targeting exon 50 at a distance of 15 nucleotides from the acceptor site (start of the exon) showed the highest exon 50 skipping activities and highest levels of dystrophin restoration at the concentration of 30 μM in AEx51 DMD patient-derived skeletal muscle cells.

### Example 5: HEx50_Ac17_24 Induces Exon 50 Skipping Activities and Dystrophin Restoration in Primary Healthy Cells and Primary DMD-Patient Derived Cells

### Cell Culture

Three healthy primary skeletal muscle cells (W018: MB07: MB09) were obtained from the University of Rochester (NY), Centre de Resources Biologiques (CBC BioTec). Primary skeletal muscle cells derived from DMD patients with a deletion of exon 51 in the DMD gene (DMD del51 cells) were obtained from Carlo Besta Neurological Institute (Milan, Italy) (Cell ID; 44328: DMD primary #1) and National Center of Neurology and Psychiatry (Tokyo, Japan) (Cell ID; 97353: DMD primary #2).

Primary cells were grown in skeletal muscle growth media (Promocell, Cat C-23160), 10 ng/ml EGF (Stemcell Technologies) and plated on 1% Matrigel coated 24-well plates (20000 cells/well). Myoblasts were induced to form myotubes in DMEM+Glutamax (Gibco) supplemented with Skeletal Muscle Cell Differentiation Medium Supplement Mix (Promocell, Cat C-39366) and 1% Pen/Strep for 2 days according to the manufacturer's instructions. PMOs were formulated in water, heated at 65-70° C., for 5-10 minutes, diluted into warm medium. Cells were harvested 48 h post transfection. Cells were collected in Trizol and stored at −80° C. until processing for RNA isolation using Direct-zol-96 RNA isolation kit (Zymo) according to the manufacturer's instructions. Total RNA concentration was quantified spectroscopically, cDNA was prepared from 200 ng of purified RNA using the High-Capacity cDNA Reverse Transcription Kit (Applied Biosciences) in a SimpliAmp Thermal Cycler (Applied Biosystems). cDNA was partitioned into droplets, in triplicate, in the QX200 Automated Droplet Generator (BioRad) in combination with Taqman probes (Thermo Fisher), 2X ddPCR Supermix (no dUTP) (BioRad), and BamHI restriction enzyme (BioRad). Following droplet generation, the mixture was loaded into a deep well C1000 Touch Thermal Cycler (BioRad) for PCR amplification. Absolute quantification of the target RNA molecules was measured in the QX200 Droplet Digital PCR System (BioRad) using the QX Manager software (BioRad). Percent Exon Skipping was calculated by normalizing the counts of the targeted exon to the total gene expression. Primers used: hDMD TaqMan assay Hs01049401_ml (VIC-MGB. Thermo Fisher Scientific) or a custom-made TaqMan assay specific for the hDMD exon 49/51 junction (FAM-MGB. Forward; 5′-TCTAAAGGGCAGCATTTGT-3″ (SEQ ID NO:80), Reverse; 5′-GGAGATGGCAGTTTCCTTAG-3′ (SEQ ID NO:81). Probe; 5′-TCAGCCAGTGAAGCTCCTACTCAGA-3′ (SEQ ID NO:82), or a custom-made TaqMan assay for the hDMD exon 49-52 junction (FAM-MGB. Forward; 5′-GCAGTTCAAGCTAAACAACCG-3′ (SEQ ID NO; 86). Reverse; 5′-TTGTTGCCTTCACTGGCT-3′ (SEQ ID NO; 87), Probe; 5′-TGTCTAAAGGGCAGCATTTGTACAAGGA-3′ (SEQ ID NO; 88). Following target amplification, samples were read in QX200 Droplet Reader (Bio-Rad. Cat. No. 1864100) for absolute quantification of target mRNA transcripts. Data were analyzed using the QX Manager software (version 1.2. BioRad). Exon skipping data are presented as percent ratio of skipped DMD transcripts and total DMD transcripts.

### Dystrophin Quantification by Jess Western System

Protein lysates were prepared and normalized based on total protein concentrations determined using the BCA method. Dystrophin restoration was quantified using the Simple Western Automated Western Blot System (ProteinSimple. Cat. No. 004-650) as per the manufacturer's instructions. The separation of proteins was achieved using a 66-440 kDa Separation Module (ProteinSimple. Cat. No. SM-W008). Dystrophin was detected using the Anti-Rabbit Detection Module (ProteinSimple. Cat. No. DM-001) with a rabbit monoclonal anti-dystrophin antibody (Abcam ab154168) diluted 1:1000 in Antibody Dilution Buffer (ProteinSimple. Cat. No. 042-203). Data were analyzed using Compass for SW software (6.2.0). The automatic peak detection feature was used to identify dystrophin peaks. Resulting electropherograms were reviewed and manual corrections were applied made if necessary. Dystrophin peaks were quantified by calculating the area under the curve (AUC). Dystrophin AUC was normalized to total protein AUC (total protein detection module). The software criteria for distinguishing low dystrophin signals from background included a peak signal-to-noise (S/N) ratio≥10 and a peak height/baseline ratio≥3, following a protocol adapted from Beekman et al., 2018.

Data are normalized to total protein and presented as percent dystrophin expression relative to healthy myotubes (AB1167) dystrophin levels (mean (technical duplicates or triplicates)±SEM).

### Results

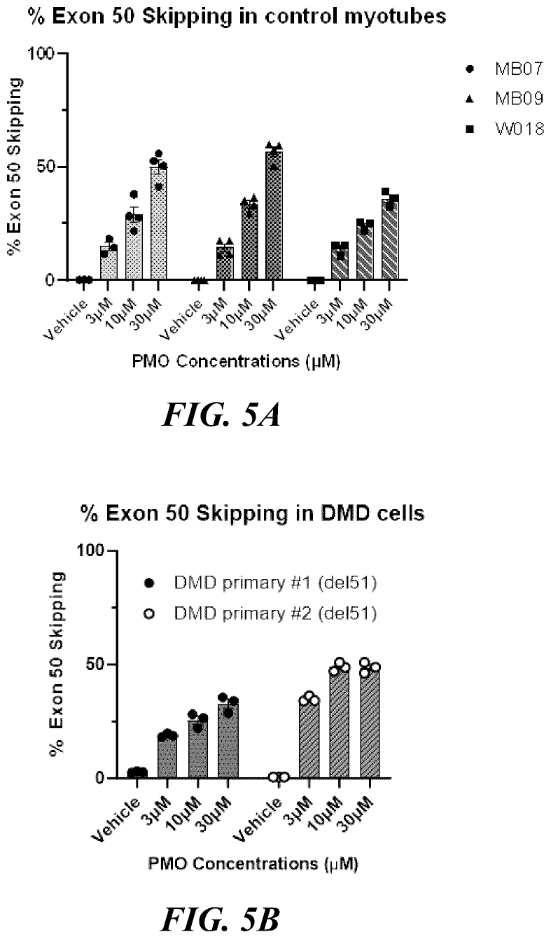

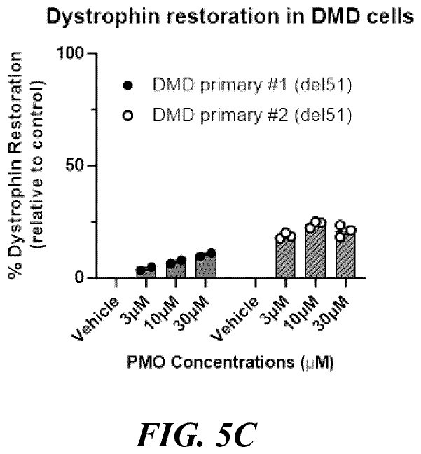

Exon 50 skipping activity was evaluated in primary healthy cells and primary DMD patient-derived cells using Digital droplet PCR (ddPCR), which is a sensitive and highly accurate method for exon skipping quantification. Myotubes derived from healthy human donors and myotubes derived from DMD patients harboring deletion of exon 51 which are amenable to exon 50 skipping therapy were treated with hEx50_Ac17_24 PMOs. (FIGS. 5A-C) A dose-dependent exon 50 skipping activity upon increased concentrations of hEx50_Ac17 24 PMOs was observed in 3 different healthy human cells (FIG. 5A) and in 2 different DMD patient-derived cells (FIG. 5B).

In addition, dystrophin restoration levels were evaluated in primary DMD-patient derived cells using western blot using the Protein Simple Jess system. A dose-dependent dystrophin restoration levels upon increased concentrations of hEx50_Ac17_24 PMOs was observed in 2 different DMD patient-derived cells (FIG. 5C).

Overall, levels of exon 50 skipping activities induced by hEx50_Ac17_24 were similar in the DMD patient-derived cells compared to the levels of healthy cells. Increases in the levels of exon 50 skipping activities correlate with increases in the levels of dystrophin restoration in the DMD-patient derived cells.

### Example 6: HEx50_Ac17_24 and hEx_50_Ac77_24 Induce Dystrophin Restoration in DMD-Patient Derived Myotubes

### Cell Culture Conditions

Three healthy primary skeletal muscle cells (W018: X819: AA179) were obtained from CBL-HCL (Centre de Resources Biologiques (CBL)-Hospices Civils de Lyon (HCL) (Lyon, France)). Primary skeletal muscle cells derived from DMD patients with a deletion of exon 51 in the DMD gene were obtained from Carlo Besta Neurological Institute (Milan, Italy) (Cell ID: 44328). Human myoblasts from DMD patients (44328) were amplified in Skeletal Muscle Growth medium (GM: Zenbio). At day 0. 15.000 of human primary myoblasts per well were seeded in GM in 96-well MyoScreen R: plates (CYTOO) coated with 10 μg/ml fibronectin (Invitrogen). Cell differentiation was induced after 24 hours of culture by changing the culture medium into differentiation medium (DM) composed of Dulbecco's Modified Eagle Medium: Nutrient Mixture F12 (DMEM/F12: Invitrogen). 2% horse serum (HS: GE Healthcare), 100 U/ml penicillin, and 100 μg/ml streptomycin (Invitrogen). At day 3, hEx50_Ac17_24 or hEx50) Ac77-24 PMOs were added to the DMD myotubes and maintained until day 8 post-treatment. For PMO treatment, hEx50)_Ac17_24 or hEx50_Ac77_24 PMOs were heated for 5 minutes at 70° C., then cooled down slowly before addition to the medium. In this specific condition Endo-Porter (GeneTools) was added simultaneously to the wells at 1 μM as delivery reagent. For each experiment, a mock condition corresponding to vehicle+/−EndoPorter was included to be used as negative control. At day 9, myotubes were fixed with formalin and permeabilized for 15 minutes with 0.5% Triton in PBS.

### Immunofluorescence Assay

For immunofluorescence staining and dystrophin quantification, cells were blocked with 1% BSA, then incubated 2 hours at room temperature with primary antibodies prepared in blocking solution: myotubes were stained with a troponin-T specific antibody (ab45932; Abcam) or a myosin heavy chain specific antibody (14-6503-82: Thermo Fisher). N-terminal dystrophin was labeled using NCL-DysB antibody (Leica), and C-terminal dystrophin was labeled using an anti-dystrophin antibody (ThermoFisher, cat #MA5-32565). Cells were incubated for 2 hours at room temperature with the corresponding secondary antibodies in combination with Hoechst 33342 (Thermo Fisher Scientific. Courtaboeuf. France). Images of cells were acquired with the Operetta HCS platform (Perkin Elmer. Villebon sur Yvette, France) using a ×10 objective, with 11 fields acquired per well. Image processing and analysis were performed using dedicated algorithms developed on the Acapella High Content Imaging Software (Perkin Elmer. Villebon sur Yvette. France) by CYTOO. Myotube and nuclei segmentation was performed using the troponin-T or myosin heavy chain (MHC) staining intensity and the Hoechst staining. The segmentation threshold was selected to avoid the detection of background noise and eliminate aberrant structures, and myotube area and dystrophin mean intensity were calculated. Three healthy donors were included in each experiment (three primary myoblast cells from CYTOO-W018). The mean intensity of dystrophin expression in these healthy myotubes was used as a reference.

### Results

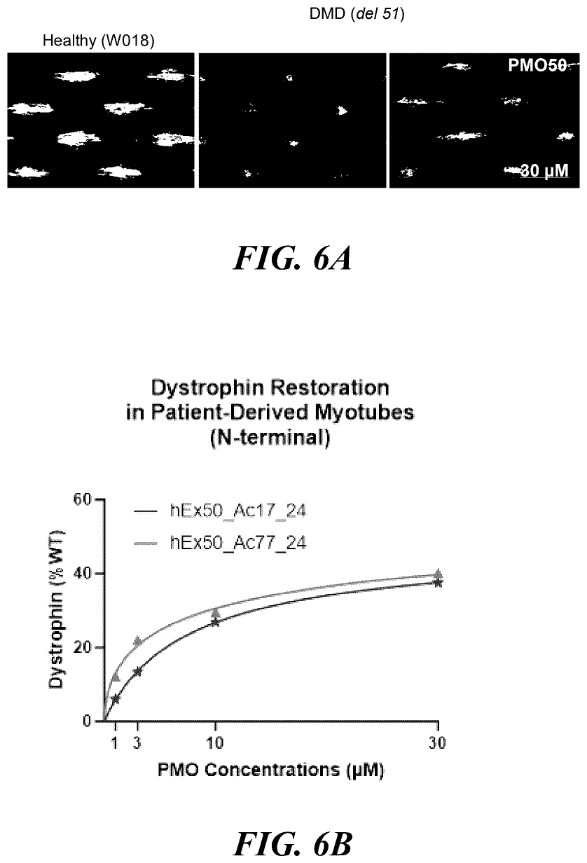

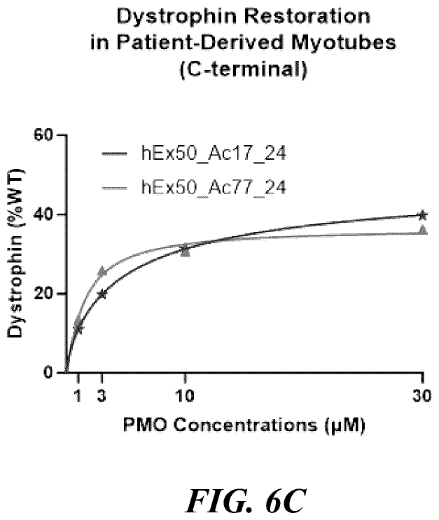

The dystrophin restoration assay was performed using the MyoScreen platform. This platform uses optimized culture conditions for differentiation, maturation, and longevity of cultured myotubes, and allows the determination of dystrophin restoration by immunofluorescence. FIG. 6A shows pictures of healthy cells and primary DMD patient-derived cells (deletion of exon 51) on the MyoScreen platform that were immunofluorescently labeled for dystrophin positive fibers. Healthy cells (left panel) and DMD patient-derived cells transfected with hEx50_Ac17_24 (right panel) showed presence of dystrophin as indicated by the positive cellular immunofluorescence staining while the mock-treated DMD patient derived cells did not express any dystrophin as evidenced by the lack of immunofluorescence staining (central panel).

In addition, the quantitative analysis of immunofluorescence staining for dystrophin restoration in primary DMD patient-derived cells transfected with increasing concentrations of hEx50_Ac17_24 PMO or hEx50_Ac77_24 show that both PMOs efficiently restored dystrophin in a dose-dependent manner in primary myotubes derived from a DMD patient that carry exon 51 deletion (FIGS. 6B-C). At a concentration of 30 μM, the hEx50_Ac17_24 PMO or hEx50)_Ac77_24 was able to restore in the range of 40% of dystrophin in primary DMD-patient derived cells compared to that of healthy primary cells (W018, X819, AA179). Therefore, treatment with hEx50_Ac17_24 or hEx50_Ac77_24 PMO resulted in exon 50 skipping activity to increase and/or restore dystrophin protein levels in vitro.

Overall, the hEx50_Ac17_24 or hEx50_Ac77_24 PMO induced similar levels of dystrophin restoration in primary DMD patient-derived cells with deletion of exon 51 as measured by cellular immunofluorescence.

Example 7: Tissue Concentrations of hEx50_Ac17_24 (PMO50) in Muscle Tissues of Humanized DMD Transgenic Mice that have been Administered with a Single Dose of DAR 4 hEx50_Ac17_24 (PMO50) AOC or DAR 10 hEx50_Ac17_24 (PMO50) AOC at Day 0) corresponding to the PMO50 dose level of 30 mg/kg
Humanized DMD (hDMD) Transgenic Mouse Model

Humanized DMD (hDMD) transgenic mice (Tg (DMD) 72Thoen/J. RRID: IMSR_JAX; 018900) were acquired from The Jackson Laboratory. The humanized DMD transgenic mouse model carries the full-length human DMD gene and expresses both mouse and human dystrophin (P. A. C. ‘T Hoen et al., “Generation and characterization of transgenic mice with the full-length human DMD gene.” Journal of Biological Chemistry, vol. 283, no. 9, pp. 5899-5907. February 2008, doi; 10.1074/jbc.M709410200).

Synthesis and Purification of DAR 4 hEx50_Ac17_24 AOC

An anti-mouse transferrin receptor IgG2a antibody was produced. The hEx50_Ac17 24 PMO was synthesized by GeneTools. Antibody (12.56 mg/ml) in phosphate buffered saline (137 mM sodium chloride, 2.7 mM potassium chloride, 10 mM disodium phosphate, 1.8 mM Monopotassium phosphate, pH 7.4) was reduced by adding ethylenediaminetetraacetic acid (0.5 mM final concentration) and 2.3 equivalents of tris (2-carboxyethyl) phosphine (TCEP) in water and incubating at 37° C., for 4 hours. 6-Maleimidocaproic acid-PFP ester (MC) was coupled to the secondary amine on the 3′ end of the hEx50_Ac17_24 PMO by incubating the hEx50_Ac17_24 PMO (50 mg/ml) in DMSO with 3 equivalents of MC (100 mg/ml) in DMSO and 3 equivalents of N,N-Diisopropylethylamine for one hour. The PMO-linker reaction was quenched by addition of cold acetate buffer (10 mM sodium acetate, pH 5.0 at 4° C.) and unconjugated MC linker was removed using Amicon Ultra-15 centrifugal filter units with a MWCO of 3 kDa by washing five times with cold acetate buffer. The PMO-MC was used immediately or stored at −20° C., to avoid maleimide hydrolysis. The reduced antibody was mixed with 5 equivalents of hEx50_Ac17_24 PMO-MC and incubated for 1 hour. The reaction was quenched with 10 equivalents of N-Ethylmaleimide at room temperature for 30 minutes to quench unreacted cysteines.

Excess PMO and NEM were removed via SCX purification (GE SP/HP 16 10 resin) using SCX method-1 with an AKTA Explorer FPLC. The combined fractions were buffer exchanged via Amicon Ultra-15 centrifugal filter units with a MWCO of 50 kDa into PBS and concentrated to approximately 25 mg Ab/mL. The solution was sterile filtered with a 0.22 μm membrane.

Strong cation chromatography (SCX) method-1
Column: GE HiPrep, SP HP 16/10, 20 ml
Solvent A; 25 mM acetate, 25 mM PB, pH 6; Solvent B; 25 mM acetate, 25 mM PB, pH 6, 0.5
mM NaCl: Flow Rate; 5 ml/min

Gradient:

| % A | % B | Column Volume |
|---|---|---|
| 100 | 0 | 3 |
| 40 | 60 | 1.5 |
| 0 | 100 | 0.2 |
| 0 | 100 | 1 |

hEx50_Ac17_24 AOC was quantified via BCA and analyzed by RCGE (average DAR˜ 3.9), SEC (2.5% HMW), LAL (<0.025 EU/mg anti-transferrin receptor antibody), and the TfR1 binding assessed by ELISA (87 pM: parent mAb=55 pM). The product was stored at 4° C.

### Synthesis and purification of DAR10 hEx50_Ac17_24 AOC

An anti-mouse transferrin receptor IgG2a antibody was produced. The hEx50_Ac17_24 PMO was synthesized by GeneTools. Antibody (12.56 mg/ml) in PBS (137 mM sodium chloride, 2.7 mM potassium chloride, 10 mM disodium phosphate, 1.8 mM Monopotassium phosphate, pH 7.4) was reduced by adding ethylenediaminetetraacetic acid (0.5 mM final concentration) and 8 equivalents of tris (2-carboxyethyl) phosphine (TCEP) in water and incubating at 37° C., for 2 hours. 6-Maleimidocaproic acid-PFP ester (MC) was coupled to the secondary amine on the 3′ end of the hEx50_Ac17_24 PMO by incubating the hEx50_Ac17_24 PMO (50 mg/ml) in DMSO with 3 equivalents of MC (100 mg/ml) in DMSO and 3 equivalents of N,N-Diisopropylethylamine for one hour. The PMO-linker reaction was quenched by addition of cold acetate buffer (10 mM sodium acetate, pH 5.0 at 4° C.) and unconjugated MC linker was removed using Amicon Ultra-15 centrifugal filter units with a MWCO of 3 kDa by washing five times with cold acetate buffer. The PMO-MC was used immediately or stored at −20° C., to avoid maleimide hydrolysis. The reduced antibody was mixed with 11 equivalents of hEx50_Ac17 24 PMO-MC and incubated for 1 hour. The reaction was quenched with 10 equivalents of N-Ethylmaleimide at room temperature for 30 minutes to quench unreacted cysteines.

Excess PMO and NEM were removed via SCX purification (GE SP/HP 16 10 resin) using SCX method-1 with an AKTA Explorer FPLC. The combined fractions were buffer exchanged via Amicon Ultra-15 centrifugal filter units with a MWCO of 50 kDa into PBS and concentrated to approximately 25 mg Ab/mL. The solution was sterile filtered with a 0.22 μm membrane.

Strong cation chromatography (SCX) method-1
Column: GE HiPrep, SP HP 16/10, 20 ml
Solvent A; 25 mM acetate, 25 mM PB, pH 6: Solvent B; 25 mM acetate, 25 mM PB, pH 6, 0.5
mM NaCl: Flow Rate; 5 ml/min

Gradient:

| % A | % B | Column Volume |
|---|---|---|
| 100 | 0 | 3 |
| 40 | 60 | 1.5 |
| 0 | 100 | 0.2 |
| 0 | 100 | 1 |

hEx50_Ac17_24 AOC was quantified via BCA and analyzed by HIC (average DAR˜ 10.1), SEC (3.2% HMW), LAL (<0.025 EU/mg anti-transferrin receptor antibody), and the TfRI binding assessed by ELISA (84 pM: parent mAb=55 pM). The product was stored at 4° C.

### Mouse In Vivo Study

Mouse studies were conducted in accordance with protocols approved by the local Institutional Animal Care and Use Committee (IACUC), following guidelines outlined in the USDA Animal Welfare Act and the “Guide for the Care and Use of Laboratory Animals” (National Research Council publication, 8th Ed., revised in 2011).

A single dose of DAR 4 hEx50_Ac17_24 AOC (DAR 4 PMO50 AOC),DAR10 hEx50_Ac17_24 AOC (DAR 10 PMO50 AOC), or PBS was administered to mice via intravenous (IV) bolus injection in the tail vein corresponding to a PMO dose of 30 mg/kg body weight in a dosing volume of 5 mL/kg body weight. Animals were humanely euthanized via CO₂ asphyxiation 14 days post-dose. Tissue necropsy samples were collected immediately after euthanasia. Skeletal muscles (gastrocnemius, vastus lateralis, and diaphragm) and cardiac muscle (whole heart) were collected at necropsy. Tissue samples weighing approximately 20-30 mg were collected in 96-well collection microtubes containing a stainless-steel bead, frozen on dry ice, and stored at −80° C. until subsequent analysis of exon skipping and tissue concentration.

### PMO Tissue Concentrations Assay

Transgenic DMD mice received a single intravenous (IV) bolus injection of DAR 4 PMO50 (hEx50_Ac17_24) AOC or DAR 10 PMO50 (hEx50_Ac17_24) AOC corresponding to a PMO dose level of 30 mg/kg. Muscle tissue samples were obtained from hDMD transgenic mice on day 14, and 4 animals per group were analyzed. A hybridization-based assay was used to quantify total hEx50_Ac17_24 (PMO50) concentration in tissue. Tissue samples of 25-45 mg were homogenized in RIPA buffer on an OMNI Bead Ruptor Elite. Calibration standards for an 8-point standard curve were generated by serial dilution of DAR 4 and DAR 10 PMO50 AOC into pooled homogenate of pre-dose gastrocnemius and vastus lateralis samples. DAR 4 and DAR10 PMO50 AOC standards and study samples were digested with 200 μg/mL proteinase K overnight at 60° C. A final round of homogenization was performed following digestion. The calibration standards and study samples were diluted similarly in assay diluent to ensure that sample values fell within the linear range of the standard curve and then incubated in 5 nM hEx_50_Ac17_24 DNA probe in hybridization buffer to allow for hybridization of hEx_50_Ac17_24 to the probe. MSD assay plates were washed and blocked, then hybridized samples were added to the assay plates. The plates were sealed and incubated to allow biotin on the probe to bind to streptavidin in the well. Plates were washed, then 6 U/mL MNase in MNase buffer was added to each well and incubated to remove un-hybridized probe from the plate. Plates were washed and detection buffer containing 0.5 μg/mL Ruthenylated detection antibody was added to each well, followed by incubation. Plates were washed, then read buffer was added to each well, followed by immediate reading on MSD Sector S 600 plate reader. The standard curve of ECL units vs. log base 10 of corresponding hEx_50_Ac17_24 PMO concentrations was generated in GraphPad Prism and fitted using a 5-parameter logistic (5-PL) fit equation (with 1/2 weighting). Study sample concentrations were interpolated from the standard curve equation and normalized based on tissue weight for tissue concentration calculation. The lower limit of quantification is 4.83 ng/ml or 0.51 nM. Tissue concentrations data are shown as mean+/−SEM. Statistical significance was assessed using ANOVA with Dunnett's Multiple Comparisons test (*p<0).05). Statistical significance was defined as p<0.05 and represented by an asterisk (*).

### Results

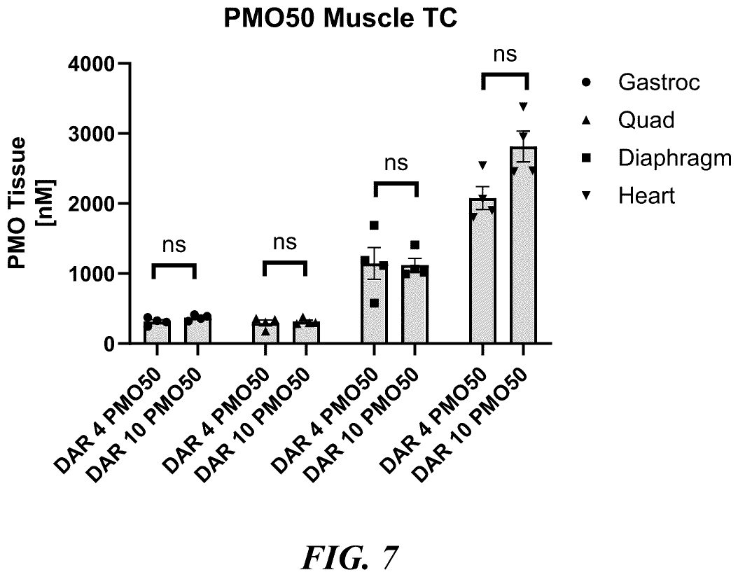

Humanized DMD (hDMD) transgenic mice received a single IV bolus injection of DAR 4 PMO50 (hEx50_Ac17_24) AOC or DAR 10 PMO50 (hEx50_Ac17_24) AOC at Day ( ) corresponding to a PMO50 dose level of 30 mg/kg. Mean total PMO concentrations were measured in muscle tissue obtained from hDMD mice at Day 14 post dose as measured by hybridization assays (FIG. 7). Among muscle tissues, cardiac muscle tissues had the highest PMO tissue concentrations in the range of 2000-3000 nM while the PMO concentrations in gastrocnemius muscle, quadriceps, diaphragm were much lower (FIG. 7). DAR 4 PMO50) AOCs and DAR 10 PMO50) AOCs delivered similar amounts of PMO50 molecules to gastrocnemius muscle and quadriceps muscle tissues.

Overall, DAR 4 PMO50) AOC and DAR 10 PM050 AOCs delivered PMO to muscle tissues. In muscle tissues, DAR 4 PM050 AOCs and DAR 10 PMO50 AOCs delivered much higher amounts of PMO50) to cardiac tissue than the ones for skeletal muscle tissues. Finally. DAR 10 PM050 AOC delivered similar amounts of PMO50 compared to the amounts with DAR 4 PMO50 AOC to each muscle tissue.

Example 8: Exon 50 skipping activities in muscle tissues obtained from humanized transgenic DMD mice receiving a single intravenous (IV) bolus injection of DAR 4 hEx50_Ac17_24 AOC or DAR 10 hEx50_Ac17_24 AOC at the PMO dose of 30 mg/kg
Humanized DMD (hDMD) Transgenic Mouse Model

Humanized DMD (hDMD) transgenic mice (Tg (DMD) 72Thoen/J. RRID: IMSR_JAX; 018900) were acquired from The Jackson Laboratory. The humanized DMD transgenic mouse model carries the full-length human DMD gene and expresses both mouse and human dystrophin (P. A. C. “T Hoen et al . . . “Generation and characterization of transgenic mice with the full-length human DMD gene.” Journal of Biological Chemistry, vol. 283, no. 9, pp. 5899-5907. February 2008, doi; 10.1074/jbc.M709410200).

The synthesis and purification of DAR 4 hEx50_Ac17_24 AOC and DAR 10 hEx50_Ac17_24 AOC are shown in Example 7.

Exon 50 Skipping Assay

Transgenic DMD mice received a single intravenous (IV) bolus injection of DAR 4 hEx50_Ac17_24 (PMO50) AOC or DAR 10 hEx50_Ac17_24 (PMO50) AOC corresponding to a PMO dose level of 30 mg/kg. Muscle tissue samples were obtained from transgenic mice on day 14 and 4 animals per group were analyzed.

Muscle tissue samples obtained from transgenic DMD mice ranging from 20-50 mg were homogenized in 1 ml of TRIzol (Thermo Fisher) on the OMNI Bead Ruptor Elite system (OMNI International). RNA was isolated from tissue homogenate supernatant using the Direct-zol-96 RNA kit (Zymo Research) according to the manufacturer's instructions. 250 ng of purified RNA was converted to cDNA using the High-Capacity cDNA Reverse Transcription Kit (Applied Biosystems) and SimpliAmp Thermal Cycler (Applied Biosystems), ddPCR was performed on 50 ng of cDNA in a reaction containing the commercially available Total DMD Taqman Assay (TaqMan assay ID: Hs01049401_ml VIC-MGB. Thermo Fisher), custom Skipped DMD Taqman assay specific for the hDMD exon 49/51 junction (FAM-MGB.

Forward; 5′-TCTAAAGGGCAGCATTTGT-3″ (SEQ ID NO:80). Reverse; 5′-GGAGATGGCAGTTTCCTTAG-3′ (SEQ ID NO:81). Probe; 5′-TCAGCCAGTGAAGCTCCTACTCAGA-3′ (SEQ ID NO:82)), cDNA was partitioned into droplets, in triplicate, in the QX200 Automated Droplet Generator (BioRad) in combination with Taqman probes (Thermo Fisher). 2X ddPCR Supermix for probes (no dUTP. Bio-Rad). BamHI-HF restriction enzyme (New England BioLabs), and Ambion nuclease free water (Thermo Fisher). Each sample. run in triplicates. was partitioned into droplets in the QX200 Automated Droplet Generator (Bio-Rad). Following droplet generation, samples were transferred to a C1000 Touch Thermal Cycler with 96-Deep Well Reaction Module (Bio-Rad). After PCR amplification, samples were loaded into the QX200 Droplet Reader (Bio-Rad). Data were analyzed using QX Manager Software. Standard Edition. Version 1.2 (Bio-Rad). Discrimination between positive and negative droplets was achieved by manually applying a fluorescence amplitude threshold. Percent exon skipping was calculated as 100*(number of skipped exon 50 DMD copies per total DMD copies per ng of cDNA). Data are presented as mean percent exon 50 skipped (mean (triplicates)±SEM). Statistical significance was assessed using ANOVA with Dunnett's Multiple Comparisons test (*p<0.05). Statistical significance was defined as p<0.05 and represented by an asterisk (*).

### Results

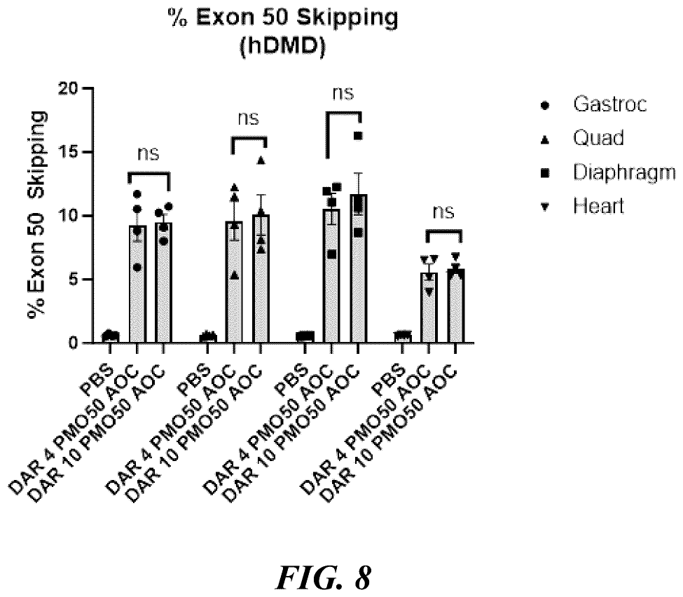

DAR 4 hEx50_Ac17_24 AOC or DAR 10 hEx50_Ac17_24 AOC was evaluated in humanized DMD (hDMD) transgenic mice. After 14 days post IV bolus injection of a single dose of DAR 4 hEx50_Ac17_24 AOC or DAR 10 hEx50_Ac17_24 AOC, muscle tissues from the animals were collected and the levels of exon 50 skipping activities in these tissues were measured by ddPCR. The results revealed that both DAR 4 hEx50_Ac17_24 AOC or DAR 10 hEx50_Ac17_24 AOC induced DMD exon 50 skipping in skeletal muscle tissues (gastrocnemius, quadricep and diaphragm) and cardiac tissue (heart) (FIG. 8). Exon 50 skipping activities induced by hEx50_Ac17_24 AOC in skeletal muscles were significantly higher than that of cardiac muscles. Levels of Exon 50 skipping activities in skeletal muscle tissues were about 10% while levels of exon 50 skipping activities were lower in cardiac tissue at around 5%.

Overall, the administration of a single dose of DAR 4 hEx50_Ac17_24 AOC or DAR 10 hEx50_Ac17_24 AOC induced higher exon 50 skipping activity in skeletal muscle tissue than the ones in cardiac muscle tissue in a humanized DMD transgenic animal model. DAR 4 hEx50 Ac17 24 AOC has similar exon 50 skipping activity as the ones for DAR 10 hEx50_Ac17_24 AOC.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A phosphorodiamidate morpholino oligonucleotide (PMO) conjugate comprising an anti-transferrin receptor antibody or antigen binding fragment thereof conjugated to a PMO molecule that hybridizes to a pre-mRNA transcript of the DMD gene and induces exon 50 skipping in said pre-mRNA transcript to generate a mRNA transcript encoding a truncated dystrophin protein, wherein the PMO molecule comprises a nucleic acid sequence having at least 90%, 95%, or 100% sequence identity any one of SEQ ID NOs; 100-169.

2. The PMO conjugate of claim 1, wherein the PMO molecule comprises a nucleic acid sequence having at least 20, 21, 22, 23, 24 nucleotides from a nucleic acid sequence selected from a group consisting of SEQ ID NOs; 100-169 with no more than one, two, three, or 4 mismatches, or consists of a nucleic acid sequence selected from a group consisting of SEQ ID NOs; 100-169.

3. The PMO conjugate of claim 1, wherein the PMO molecule comprises a nucleic acid sequence selected from SEQ ID NOs; 144-169.

4. The PMO conjugate of claim 1, wherein the PMO molecule comprises or consists of a nucleic acid sequence selected from SEQ ID NOs; 148-160.

5. The PMO conjugate of claim 1, wherein the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof.

6. The PMO conjugate of claim 1, wherein the PMO molecule is conjugated to the anti-transferrin receptor antibody or antigen binding fragment thereof via a linker.

7. The PMO conjugate of claim 6, wherein the linker is a cleavable linker.

8. The PMO conjugate of claim 6, wherein the linker is a non-cleavable linker.

9. The PMO conjugate of claim 6, wherein the linker is selected from the group consisting of a heterobifunctional linker, a homobifunctional linker, a linker comprising a maleimide group, a dipeptide moiety, a benzoic acid group or derivatives thereof, a C₁-C₆ alkyl group, and a combination thereof.

10. The PMO conjugate of claim 1, wherein the PMO conjugate has a PMO molecule to antibody ratio (DAR) of about 1:1, 2:1, 3:1, 4:1 5:1, 6:1, 7:1, 8:1, 9:1, 10:1 or higher.

11. The PMO conjugate of claim 1, wherein the PMO conjugate has a DAR of about 4.

12. The PMO conjugate of claim 1, wherein the PMO conjugate has a DAR of about 10.

13. A method of treating muscular dystrophy in a subject in need thereof comprising administering to said subject a phosphorodiamidate morpholino oligonucleotide (PMO) conjugate comprising an anti-transferrin receptor antibody or antigen binding fragment thereof conjugated to a PMO molecule; wherein the PMO molecule hybridizes to a site within an exon, an acceptor splice site, a donor splice site, or an exonic splice enhancer element of a pre-mRNA transcript of the DMD gene and induces exon 50 skipping in said pre-mRNA transcript to generate a mRNA transcript encoding a truncated dystrophin protein, thereby treating muscular dystrophy.

14. The method of claim 13, wherein the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof.

15. The method of claim 13, wherein the PMO molecule comprises a nucleic acid sequence having at least 90%, 95%, or 100% sequence identity any one of SEQ ID NOs; 100-169, or wherein the PMO molecule comprises a nucleic acid sequence having at least 20, 21, 22, 23, 24 nucleotides from a nucleic acid sequence selected from a group consisting of SEQ ID NOs; 100-169 with no more than one, two, three, or 4 mismatches, or consists of a nucleic acid sequence selected from a group consisting of SEQ ID NOs; 100-169.

16. The method of claim 13, wherein the PMO molecule comprises a nucleic acid sequence selected from SEQ ID NOs; 144-169.

17. The PMO conjugate of claim 13, wherein the PMO molecule comprises or consists of a nucleic acid sequence selected from SEQ ID NOs; 148-160.

18. The method of claim 13, wherein the PMO conjugate has an average of PMO molecule to antibody ratio (DAR) of about 1:1, 2:1, 3:1, 4:1, 5:1, 6:1, 7:1, 8:1, 9:1, 10:1.

19. The method of claim 13, wherein the PMO conjugate is administered parenterally.

20. The method of claim 13, wherein the muscular dystrophy is Duchenne muscular dystrophy or Becker muscular dystrophy.
